(19) United States
(12) Patent Application Publication
(10) Pub. No.: US 2005/0123476 A1
(43) Pub. Date: Jun. 9, 2005

(54) Imaging the activity of extracellular protease in cells using mutant anthrax toxin protective antigens that are cleaved by specific extracellular proteases

(75) Inventors: Thomas H. Bugge, Bethesda, MD (US); Stephen H. Leppla, Bethesda, MD (US); Shi-Hui Liu, Rockville, MD (US); David Mitola, Baltimore, MD (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: The Government of the United States as represented by the Secretary of the Department of Health and, Rockville, MD (US)

(21) Appl. No.: 10/488,806

(22) PCT Filed: Sep. 5, 2002
(86) PCT No.: PCT/US02/28397

Related U.S. Application Data
(60) Provisional application No. 60/317,550, filed on Sep. 5, 2001.

(51) Int. Cl. A61K 51/00; A61K 49/00; C12Q 1/37

(52) U.S. Cl. 424/1.49; 424/9.34; 424/9.6; 435/23

(57) ABSTRACT

This invention pertains to methods for imaging the activity of extracellular proteases in cells using the anthrax binary toxin-system to target cells expressing extracellular proteases with mutant anthrax toxin protective antigens (μPrAg) that bind to receptors on the cells and are cleaved by a specific extracellular protease expressed by the cells, and ligands that specifically bind to the cleaved μPrAg and are linked to a moiety that is detectable by an imaging procedure. The μPrAg proteins used in the methods comprise a protease cleavage site that is cleaved by a specific extracellular protease and is in place of the furin cleavage site of the native PrAg. The methods are useful for diagnosing and treating diseases and undesirable physiological conditions correlated with the activity of extracellular proteases, and for optimizing the therapeutic efficacy of drugs used to treat such diseases and conditions.

### CROSS-REFERENCES TO RELATED APPLICATIONS

The present application claims priority to U.S. Ser. No. 60/317,550, filed Sep. 5, 2001, herein incorporated by reference in its entirety.

### STATEMENT AS TO RIGHTS IN INVENTIONS MADE UNDER FEDERALLY SPONSORED RESEARCH AND DEVELOPMENT

Not applicable.

### BACKGROUND OF THE INVENTION

1. Field of the Invention

This invention pertains to methods for imaging the activity of extracellular proteases in cells using the anthrax binary toxin system to target cells expressing extracellular proteases with mutant anthrax toxin protective antigens (μPrAg) that bind to receptors on the cells and are cleaved by a specific extracellular protease expressed by the cells, and ligands that specifically bind to the cleaved μPrAg and are linked to a moiety that is detectable by an imaging procedure.

2. Background

Numerous studies have demonstrated a positive correlation between the activity of extracellular proteases and various diseases and undesirable physiological conditions such as cancer, autoimmune disease, cardiovascular disease, inflammation, infection, and neurological disorders. For example, the dissolution of the extracellular matrix by extracellular proteases is a prerequisite for the invasive growth of malignant cells, metastatic spread of tumors, and physiological remodeling of tissue. Matrix dissolution is accomplished by the concerted effort of a number of extracellular proteases, including serine, metallo-, and cysteine proteases. For example, these extracellular proteases play a central role in the conversion of the plasma protease, plasminogen, to the active protease, plasmin (Dano et al. (1999) APMIS 107, 120-127; Andreasen et al. (2000) Cell Mol. Life Sci. 57, 25-40; Koblinski et al. (2000) GUn. C/jim. Acta 291, 113-135).

In order to effectively diagnose and treat a disease or undesirable physiological condition correlated with the activity of such extracellular proteases, a highly specific and sensitive method for detecting and monitoring the activity of the specific extracellular proteases in cells is needed.

A. Extracellular Proteases of the Plasminogen Activation System

Plasminogen is cleaved by either of two extracellular proteases, the urokinase plasminogen activator (uPA) and the tissue plasminogen activator (tPA), to form plasmin. The uPA is a 52 kDA serine protease that is secreted as an inactive single chain proenzyme (pro-uPA) that is efficiently converted to active two-chain uPA by plasmin (4). The two-chain uPA, in turn, is a potent activator of plasminogen, leading to a powerful feedback loop that results in productive plasmin formation. However, both pro-uPA and plasminogen are catalytically inactive pro-enzymes. Pro-uPA binds with high affinity (Kd₅=0.5 nM) to a specific glycosyiphosphatidylinositol-linked cell surface receptor, the uPA receptor (uPAR), via an epidermal growth factor-like amino-terminal fragment (ATF; amino acids 1-135, 15 kDa) (5). The uPAR is a 60 kDa, three-domain glycoprotein with a first and third domain that constitutes a composite high-affinity binding site for the ATF of pro-uPA (5-8). The concomitant binding of pro-uPA to uPAR, and of plasminogen to cell surface receptors, strongly potentiates uPA-mediated plasminogen activation (9-12), which may be due to the formation of ternary complexes, aligning the two proenzymes in a way that exploits their low intrinsic activity and thereby favoring a mutual activation process (13). The net result of this process is the efficient and localized generation of active uPA and plasmin on the cell surface.

Further, recent studies in uPAR-deficient mice have demonstrated the existence of additional uPAR-independent pathways of uPA-mediated plasminogen activation, in the context of both physiological cell migration and fibrin dissolution (14, 15).

The uPAR and uPA are overexpressed with remarkable consistency in malignant human tumors, including monocytic and myelogenous leukemias (16, 17) and cancers of the colon (18), breast (19), bladder (20), thyroid (21), liver (22), pleura (23), lung (24), pancreas (25), ovaries (26), and the head and neck (27). Extensive in situ hybridization and immunohistochemical studies of various human tumor types have demonstrated that cancer cells typically express uPAR, whereas pro-uPA may be expressed by either the cancer cells or by adjacent stromal cells (18, 28, 29).

Plasminogen activation by uPA is regulated by two physiological inhibitors, plasminogen activator inhibitors-1 and -2 (PAI-1 and PAI-2) (30-32), each forming a 1:1 complex with uPA. Plasmin generated by the cell surface plasminogen activation system is relatively protected from its primary physiological inhibitor α₂-antiplasmin (11, 33, 34). Unlike uPA, plasmin is a relatively nonspecific protease, capable of degrading fibrin and several other glycoproteins and proteoglycans of the extracellular matrix (35). Therefore, cell surface plasminogen activation facilitates invasion and metastasis of tumor cells by dissolution of restraining tissue barriers. In addition, cell surface plasminogen activation may facilitate matrix degradation through the activation of latent matrix metalloproteinases (MMP) (36). Plasmin can also activate growth factors, such as transforming growth factor-β, which may further modulate stromal interactions in the expression of enzymes and tumor neo-angiogenesis (37).

B. The Anthrax Toxin System

Another protein that requires cell surface proteolytic activation is anthrax toxin. Anthrax toxin is a binary toxin secreted by Bacillus anthracis consisting of protective antigen (PrAg, 83 kDa) and either lethal factor (LF, 90 kDa) or edema factor (EF, 89 kDa) (1-3). Individually, each protein is non-toxic to cells. However, the combination of PrAg and either LF or EF is toxic to cells. The mechanism by which individual toxin components interact to cause toxicity was recently reviewed (3). PrAg, the cellular receptor-binding component, binds to a cellular receptor (4); see also Bradley et al., Nature 414:225-229 (2001)) and is cleaved at the sequence RKKR₁₆₇ by cell-surface furin or furin-like proteases (5, 6) into two fragments: PA63, a 63 kDa C-terminal fragment, which remains receptor-bound; and PA20, a 20 kDa N-terminal fragment, which is released into the medium (7). Dissociation of PA20 allows PA63 to form heptamer (8, 9) and also bind LF or EF (10). The resulting hetero-oligomeric complex is internalized by endocytosis (11), and acidification of the vesicle causes insertion of the PA63 heptamer into the endosomal membrane to produce a channel through which LF or EF translocate to the cytosol (12), where LF and EF induce cytotoxic events.

Thus, the combination of PrAg and LF, named anthrax lethal toxin, kills animals (13, 14) and certain cultured cells (12, 15), due to intracellular delivery and action of LF, recently shown to be a zinc-dependent metalloprotease that is known to cleave at least two targets, mitogen-activated protein kinase 1 and 2 (16, 17). The combination of PrAg and EF, named edema toxin, disables phagocytes and probably other cells, due to the intracellular adenylate cyclase activity of EF (18).

LF and EF have substantial sequence homology in amino acid (aa) 1-250 (3), and a mutagenesis study showed this region constitutes the PrAg-binding domain (19). Systematic deletion of LF fusion proteins containing the catalytic domain of Pseudomonas exotoxin A established that LF aa 1-254 (LFn) are sufficient to achieve translocation of “passenger” polypeptides to the cytosol of cells in a PrAg-dependent process (20, 21). A highly cytotoxic LFn fusion to the ADP-ribosylation domain of Pseudomonas exotoxin A, named FP59, has been developed (21). When combined with PrAg, FP59 kills any cell type which contains receptors for PrAg by the mechanism of inhibition of initial protein synthesis through ADP ribosylating inactivation of elongation factor 2 (EF-2), whereas native LF is highly specific for macrophages (3). For this reason, FP59 is an example of a potent therapeutic agent when specifically delivered to the target cells with a target-specific PrAg.

The crystal structure of PrAg (PDB accession 1ACC) (22) indicates that it has four distinct domains (domains 1-4) that are associated with functions previously defined by biochemical analysis. Domain 1 (aa 1-258) contains two tightly bound calcium ions, and a large flexible loop (aa 162-175) that includes the sequence RKKR₁₆₇, which is cleaved by furin during proteolytic activation. Domain 2 (aa 259-487) contains several very long β-strands and forms the core of the membrane-inserted channel. It is also has a large flexible loop (aa 303-319) implicated in membrane insertion. Domain 3 (aa 488-595) has no known function. Domain 4 (aa 596-735) is loosely associated with the other domains and is involved in receptor binding. For cleavage at RKKR₁₆₇ is required for the subsequent steps in toxin action, it would be of great interest to engineer it to the cleavage sequences of some disease-associated proteases, such as matrix metalloproteases (MMPs) and plasminogen activators (e.g., t-PA, u-PA, uPAR, PA1-1, see, e.g., Romer et al., APMIS 107:120-127 (1999)), which are typically overexpressed in tumors and other diseases.

MMPs and plasminogen activators are families of enzymes that play a leading role in both the normal turnover and pathological destruction of the extracellular matrix, including tissue remodeling (23, 24), angiogenesis (25, 26), tumor invasion and metastasis formation. The members of the MMP family are multidomain, zinc-containing, neutral endopeptidases and include the collagenases, stromelysins, gelatinases, and membrane-type metalloproteases (23). It has been well documented in recent years that MMPs and plasminogen activators are overexpressed in a variety of tumor tissues and tumor cell lines and are highly correlated to the tumor invasion and metastasis (27-53).

Among the MMPs, MMP-2 (gelatinase A), MMP-9 (gelatinase B) and membrane-type 1 MMP (MT1-MMP) are reported to be most related to invasion and metastasis in various human cancers (27-53). The important role of MMPs during tumor invasion and metastasis is to break down tissue extracellular matrix and dissolution of epithelial and endothelial basement membranes, enabling tumor cells to invade through stroma and blood vessel walls at primary and secondary sites. MMPs also participate in tumor neoangiogenesis and are selectively upregulated in proliferating endothelial cells in tumor tissues (25, 26, 54). Furthermore, these proteases can contribute to the sustained growth of established tumor foci by the ectodomain cleavage of membrane-bound pro-forms of growth factors, releasing peptides that are mitogens for tumor cells and/or tumor vascular endothelial cells (55, 56).

However, catalytic manifestations of MMP and plasminogen activators are highly regulated. For example, the MMPs are expressed as inactive zymogen forms and require activation before they can exert their proteolytic activities. The activation of MMP zymogens involves sequential proteolysis of N-terminal propeptide blocking the active site cleft, mediated by proteolytic mechanisms, often leading to an autoproteolytic event (57, 58). Second, a family of proteins, the tissue inhibitors of metalloproteases (TIMPs), are correspondingly widespread in tissue distribution and function as highly effective MMP inhibitors (Ki˜10⁻¹⁰ M) (59). Though the activities of MMPs are tightly controlled, invading tumor cells that utilize the MMP's degradative capacity somehow circumvent these negative regulatory controls, but the mechanisms are not well understood.

The contributions of MMPs in tumor development and metastatic process lead to the development of novel therapies using synthetic inhibitors of MMPs (60, 61, 62). Among a multitude of synthetic inhibitors generated, Marimastat is already clinically employed in cancer treatment (62).

Recently it has been shown physiological concentrations of plasmin can activate both MMP-2 and MMP-9 on cell surface of HT1080 by a mechanism independent of MMP or acid protease activities (70). In contrast, in soluble phase plasmin degrades both MMP-2 and MMP-9 (70). Thus, plasmin may provide a mechanism keeping gelatinase activities on cell surface to promote cell invasion. It has been well established MT1-MMP functions as both activator and receptor of MMP-2, but has no effect on MMP-9 (see review 50, 53). A MMP-2/TIMP-2 complex binds to MT1-MMP on the cell surface, which serves as a high-affinity site, and is then proteolytically activated by an adjacent MT1-MMP, which serves as an activator.

C. Detection of Protease Activity

The detection of a specific extracellular protease activity is critical to the effective diagnosis and treatment of diseases and undesirable physiological conditions correlated with a specific protease activity, and for the evaluation and optimization of the therapeutic efficacy of drugs used for treatment of such diseases and conditions. However, there has been considerable difficulty in detecting and monitoring extracellular protease activity in cells. Current methods for detecting protease activity in cells rely on indirect measurements of tumor regression and progression using surrogate markers or direct measurement of tumor volume. However, such markers often do not specifically target the cells or activity of interest, and such measurements are often not statistically significant until weeks after the initiation of treatment or not indicative of the drug efficacy. Moreover, current imaging techniques rely on detectable moieties that have a low signal to noise ratio, and are not effective for detecting the activity of a specific extracellular protease in cells. Thus, there is a need for highly specific and sensitive methods for imaging the activity of specific extracellular proteases in cells. The present invention fulfills these and other needs.

The inventors are the first to appreciate that the specific requirements of PrAg cleavage provide a unique opportunity to modify this protein to make the cleavage dependent on the proteolytic activity of specific extracellular proteases expressed in cells and, therefrom, develop a novel approach for imaging the activity of these specific extracellular proteases in cells. In particular, the present invention provides highly specific and sensitive methods for imaging the activity of specific extracellular proteases in cells, useful for diagnosing and treating diseases or undesirable physiological conditions correlated with the expression of specific extracellular proteases, including cancer, autoimmune disease, cardiovascular disease, inflammation, infection, and neurological disorders, and for optimizing the therapeutic efficacy of drugs used in the treatment of such a diseases or conditions.

### SUMMARY OF THE INVENTION

The present invention provides highly specific and sensitive methods for imaging in vivo, in vitro and ex vivo the activity of a specific extracellular protease in a cell using the anthrax binary toxin system to specifically target a cell that expresses the specific extracellular protease. In particular, the present invention provides methods for imaging a specific extracellular protease by contacting a cell with: a mutant anthrax toxin protective antigen (μPrAg) that binds to a cell surface receptor of a cell expressing an extracellular protease and is cleaved by a specific extracellular protease expressed by the cell; and a ligand that specifically binds to the cleaved μPrAg and is linked to a moiety that is detected by an imaging procedure, thereby, forming a ligand-μPrAg complex. The ligand-μPrAg complex is optionally translocated into the cell, or can be a non-translocatable complex. The detectable moiety linked to the ligand in the ligand-μPrAg complex is imaged before, during, or after translocation of the complex into the cell. An image indicative of the activity of the specific extracellular protease is thereby generated. The μPrAg proteins used in the methods of the present invention have a domain for binding the cell surface receptor of a cell expressing an extracellular protease, and have a protease cleavage site that is cleaved by a specific extracellular protease expressed by the cell and is in place of the furin cleavage site of the native anthrax toxin protective antigen (PrAg).

In another embodiment of the present invention, the methods are performed in vivo, ex vivo, or in vitro. In a preferred embodiment, the methods are performed in vivo, for example in a mammal. Examples of a mammal are a rodent and a human, but are not limited thereto.

In another embodiment of the present invention, the specific extracellular protease of the methods is a matrix metalloproteinase (MMP) or plasminogen activator (PA). More particularly, the MMP is MMP-2 (gelatinase A), MMP-9 (gelatinase B), or membrane-type 1 MMP (MTI-MMP); and the PrAg is tissue plasminogen activator (tPA) or urokinase plasminogen activator (uPA).

In another embodiment of the present invention, the protease cleavage site of the μPrAg of the methods is encoded by an amino acid sequence selected the following group of sequences: GPLGMLSQ, GPLGLWAQ, PCPGRVVGG, PGSGRSA, PGSGKSA, PQRGRSA, PCPGRVVGG, PGSGRSA, PGSGKSA, PQRGRSA, GPLGMLSQ, and GPLGLWAQ.

In another embodiment of the present invention, the ligand of the methods is a protein that specifically binds to the cleaved μPrAg, for example, an antibody, noncytotoxic lethal factor, noncytotoxic edema factor, derivative of a noncytotoxic lethal factor, or derivative of a noncytotoxic edema factor, FP59, LFN, or a noncytotoxic lethal factor comprising no more than amino acids 1-254 of LF.

In another embodiment of the present invention, the detectable moiety of the methods is imaged by magnetic resonance, radioscintigraphy, positron emission tomography (PET), computed tomography (CT), near-infrared fluorescence (NIRF), X-ray, ultra sound, ultraviolet light, or visible light, but is not limited thereto. For example, the imaging procedure used to image the detectable moiety can be any imaging procedure described herein or known to one skilled in the art, and suitable for detecting the moiety linked to the ligand. The imaging of the detectable moiety can occur prior to, during, or after translocation of the ligand-μPrAg complex into the cell.

In another embodiment of the present invention, the detectable moiety of the methods is a radionuclide, metal ion, biotin, enzyme, chromophore, or fluorophore, but is not limited thereto. For example, the detectable moiety can be any moiety detectable by an imaging procedure described herein or known to one of skill in the art, and can be directly or indirectly linked to the ligand. The detectable moiety may comprise a single detectable molecule or multiple detectable molecules (for example, an array or plurality of detectable molecules) linked to the ligand.

In another embodiment of the present invention, the cell of the methods is a cancer cell, an inflammatory cell, a lymphocyte, a cardiovascular cell, or a neuron. In a preferred embodiment, the cell is in a mammal. Examples of mammals include a rodent or a human, but are not limited thereto.

In a preferred embodiment of the present invention, in the methods where the cell is in a mammal, for example, a rodent or a human, a composition comprising the μPrAg and ligand are administered to the mammal prior to contacting the cells.

In another preferred embodiment of the present invention, in the methods where the cell is in a mammal, for example, a rodent or a human, the activity of the protease is a diagnostic indicator of a disease or condition correlated with the activity of the protease. Examples of a disease or condition include cancer, metastasis, tumor regression or progression, inflammation, autoimmune disease, cardiovascular disease, infection, and neurological disorders, but are not limited thereto. The disease is any disease in which extracellular proteolysis is part of the etiology, or in which increased extracellular protease activity serves as a marker for disease or disease progression.

In another preferred embodiment, the methods of the present invention are used to monitor the efficacy of pharmacological and genetic inhibition or protease activity in the treatment of diseases having extracellular proteolysis as part of their etiology, or in which increased extracellular protease activity serves as a marker for disease or disease progression, e.g., cancer, metastasis, tumor regression or progression, inflammation, autoimmune disease, cardiovascular disease, infection, and neurological disorders.

In another preferred embodiment, the methods of the present invention are used to assay for protease inhibitors for the treatment of diseases having extracellular proteolysis as part of their etiology, or in which increased extracellular protease activity serves as a marker for disease or disease progression, e.g., cancer, metastasis, tumor regression or progression, inflammation, autoimmune disease, cardiovascular disease, infection, and neurological disorders.

### BRIEF DESCRIPTION OF THE DRAWINGS

Not applicable.

### DETAILED DESCRIPTION OF THE INVENTION AND THE PREFERRED EMBODIMENTS

The present inventors are the first to appreciate that the specific requirements of PrAg cleavage provide a unique opportunity to modify this protein to make the cleavage dependent on the proteolytic activity of a specific extracellular protease and, therefrom, develop a novel approach for imaging the activity of a specific extracellular protease in cells. In particular, the present invention is directed to highly specific and sensitive methods for imaging in vivo the activity of a specific extracellular protease in a cell using the anthrax binary toxin system to target a cell that expresses the specific extracellular protease.

More specifically, the present invention provides methods for imaging a specific extracellular protease by contacting a cell with: a mutant anthrax toxin protective antigen (μPrAg) that binds to a cell surface receptor of a cell expressing an extracellular protease and is cleaved by a specific extracellular protease expressed by the cell; and a ligand that specifically binds to the cleaved μPrAg and is linked to a moiety that is detected by an imaging procedure, thereby, forming a ligand-μPrAg complex. The complex is optionally translocated into the cell. The detectable moiety linked to the ligand of the ligand-μPrAg complex is imaged prior to, during, or after translocation of the complex into the cell and an image indicative of the activity of the specific extracellular protease is thereby generated. The μPrAg proteins of the present invention have a domain for binding the cell surface receptor of a cell expressing an extracellular protease, and have a protease cleavage site that is cleaved by a specific extracellular protease expressed by the cell and is in place of the furin cleavage site of the native PrAg.

The methods of the present invention may be used, for example, to optimize the efficacy of a drug used in the treatment of a disease or undesirable physiological condition correlated with the activity of a specific extracellular protease expressed by a cell. Also, the methods may be used, for example, to diagnose and monitor the treatment of a disease or undesirable physiological condition correlated with the activity of a specific extracellular protease.

Further, the methods of the present invention may be used, for example, to deliver therapeutically effective compounds linked to the ligand for treatment of a disease or undesirable physiological condition correlated with the activity of a specific extracellular protease. Thus, for example, the detectable moieties themselves may have therapeutic efficacy, e.g., by virtue of the radiotherapeutic effect of a radionuclide or photodynamic effect of a chromophore (or fluorophore). In addition, the ligand and/or detectable moiety may be further linked to a therapeutic agent that modulates, inhibits, or activates an activity of the cell having a therapeutic effect. For example, the ligand and/or detectable moiety could modulate or inhibit the activity of an extracellular protease correlated with a disease or undesirable physiological condition.

The methods of the present invention may be used in vivo, ex vivo, or in vitro to detect the activity of a specific extracellular protease expressed by a cell.

Abbreviations

The abbreviations used are: APP, amino-terminal fragment of urokinase plasminogen activator; DMEM, Dulbecco's modified Eagle's medium; EF, edema factor; LFN, amino acids 1-254 of native LF; FP59, fusion protein of LF amino acids 1-254 and Pseudomonas exotoxin A domain III; HUVEC, human umbilical vein endothelial cells; LF, lethal factor; MMP, matrix metalloproteinase; MTT, 3-(4,5-dimethylthiazol-2-yl)-2,5-diphenyltetrazolium bromide; PrAg, anthrax toxin-protective antigen; PrAg2O, amino-terminal 20.kDa fragment of PrAg; PrAg63, carboxyl-terminal 63-kDa fragment of PrAg; PAGE, polyacrylamide gel electrophoresis; PAI-1, plasminogen activator inhibitor-i; PAI-2, plasminogen activator inhibitor-2; tPA, tissue plasminogen activator; uPA, urokinase plasminogen activator; uPAR, urokinase plasminogen activator receptor.

Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

The phrase “extracellular protease” refers to a protease localized on the surface of a cell, and is not limited to a protease embedded or attached, directly or indirectly, to the cell. An example of a extracellular protease is a protease capable of specifically cleaving a μPrAg used in the methods (see, e.g., WO 01/21656). Examples of extracellular proteases are serine, matrix metallo-, and cysteine proteases, but are not limited thereto. Further examples of extracellular proteases are proteases belonging to the following classes of protease: MMP (e.g., MT1-MMP), ADAMS (e.g., ADAM-15), type-I transmembrane serine proteases (e.g., prostasin), type-II transmembrane serin proteases (e.g., matriptase), cathepsins (e.g., cathepsin B), GPI-anchored serine proteases, as described in Frosch et al., APMIS (1999) 107: 28-37; Schlondorff et al., J. Cell Sci. (1999) 112: 3603-3617; Kaushal et al., J. Clinical Investigation (2000) 105: 13351337; Ellerbroek, BioEssays (1999) 21: 940-949; and Hooper et al. (2001) 276: 857-860.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Protein”, “polypeptide”, or “peptide” refer to a polymer in which the monomers are amino acids and are joined together through amide bonds, alternatively referred to as a polypeptide. When the amino acids are α-amino acids, either the L-optical isomer or the D-optical isomer can be used. Additionally, unnatural amino acids, for example, β-alanine, phenylglycine and homoarginine are also included. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

The phrase “recombinant protein” as used herein refers to a protein which has been produced by a recombinant cell.

The terms “μPrAg” or “mutant anthrax toxin protective antigen” as used herein refer to a recombinant or synthetic protein that is a modified, mutant, or derivative form of the native PrAg, that comprises a cleavage site of a specific extracellular protease in place of the furin cleavage site of the native PrAg and binds to a receptor on the surface of a cell expressing an extracellular protease. The protease cleavage site may be a native protease cleavage site, a mutant, modified, or derivative form of a native protease cleavage site, or an artificial or synthetic cleavage site, that is cleaved by a specific extracellular protease. For example, see PCT application WO 01/21656. In addition, the mutant, modified, derivative, artificial or synthetic protease cleavag site may confer an activity or specificity that differs from the native protease cleavage site, for example, an increase or decrease in protease cleavage activity or specificity. Further, the μPrAg may be constructed as a fusion protein, for example, a comprising an amino acid sequence encoding a μPrAg and a ligand (e.g., LF or EF), or subsequences thereof (see, e.g., U.S. Pat. Nos. 5,677,274 and 5,591,631).

The term “ligand,” as used herein with reference to a ligand of μPrAg, refers to a recombinant or synthetic molecule that specifically binds to a μPrAg, or is fused to the μPrAg, and is suitable for use in the methods of the present invention. Examples of suitable ligands include, lethal factor (LF), endema factor (EF), mutated, modified, or derivative forms of LF or EF, antibodies to PrAg, and other proteins (and mutated, modified, or derivative forms thereof) that bind specifically to the μPrAg used in the methods of the present invention, but are not limited thereto (see, e.g., U.S. Pat. Nos. 5,677,274 and 5,591,631). Another example of a suitable ligand is a subsequence of LF (LFN), located at approximately amino acids 1-245 of LF, which retains the ability to specifically bind PrAg and translocate (as described in Arora et al., J. Biol. Chem. 267: 15542-15548 (1992) and Arora et al., J. Biol. Chem. 268: 3334-3341 (1993)). For example, see U.S. Pat. Nos. 5,591,631 and 5,677,274. The ligand may be linked directly or indirectly to a detectable moiety. For example, the ligand may be linked indirectly to the detectable moiety using a linker as described herein. Further, the ligand may be constructed as a fusion protein comprising the ligand and a μPrAg.

The terms “mutant,” “modified,” and “derivative” refer to the manipulation, of nucleic acid sequence or amino acid sequence encoding a protein, by recombinant or synthetic methods, resulting in a change in the nucleic acid sequence or amino acid sequence, respectively, such that the sequence is different from the original or unmanipulated sequence. For example, an nucleic acid sequence or amino acid sequence encoding a protein can be manipulated by extending, shortening, replacing, or otherwise changing the original or unmanipulated, by using the recombinant or synthetic methods described herein or known to one of skill in the art.

The phrase “fusion protein” refers to a protein comprising amino acid sequences that are in addition to, in place of, less than, and/or different from the amino acid sequences encoding the original or native full-length protein or subsequences thereof. Moreover, a nucleic acid sequence or amino acid sequence of a first protein can be modified to contain sequences that are substantially identical to the nucleic acid sequence or amino acid sequence, respectively, of a second protein and, thereby, a “fusion protein” is constructed. Fusion proteins comprising the μPrAg and/or ligands used in the methods of the present invention are contemplated.

The term “imaging” refers to a procedure or modality for generating an image of a detectable moiety in vivo, ex vivo, or in vitro, as described herein or known to one of skill in the art. Examples of imaging modalities include magnetic resonance, nuclear magnetic resonance, radioscintigraphy, positron emission tomography, computed tomography, near-infrared fluorescence, X-ray, ultra sound, ultraviolet light, or visible light, but are not limited thereto (for example, see Dahnhert, Radiology Review Manual, 4 th Edition, Lippincott, Williams & Wilkins (1999); Brant et al., Fundamentals of Diagnostic Radiobiology, 2 nd Edition, Lippincott, Williams & Wilkins (1999); Weissleder et al., Primer of Diagnostic Imaging, 2 nd Edition, Mosby-Year Book (1997); Buddinger et al., Medical Magnetic Resonance A Primer, Society of Magnetic Resonance, Inc. (1988); and Weissleder et al., Nature Biotech. 17: 375-378 (1999)). In a preferred embodiment, the image of the detectable moiety is indicative of the activity of a specific extracellular protease.

The phrase “detectable moiety” as used herein refers to a moiety that can be imaged and/or detected in vivo, ex vivo, or in vitro, by a procedure or modality described herein or known to one of skill in the art. As used herein, the detectable moiety can be directly or indirectly linked to the ligand used in the methods of the present invention.

The phrases “enzymatic activity” or “binding activity” or “protease activity” or “cleavage activity” as used herein refer to the activity of a biomolecule, for example a protein, and may be measured by a variety of assays and units as described herein or known to one skilled in the art.

The phrase “catalytic domain” refers to a protein domain, or portion thereof, that is sufficient to catalyze an enzymatic reaction that is normally carried out by the enzyme.

The term “subsequence” refers to a sequence of nucleic acids or amino acids that comprise a part of a longer sequence of nucleic acids or amino acids (e.g., protein) respectively.

The phrase “nucleic acid” refers to a deoxyribonucleotide or ribonucleotide polymer in either single-or double-stranded form, and unless otherwise limited, encompasses known analogues of natural nucleotides that hybridize to nucleic acids in manner similar to naturally occurring nucleotides. Unless otherwise indicated, a particular nucleic acid sequence includes the complementary sequence thereof.

The phrases “recombinant expression cassette” or simply an “expression cassette” refer to a nucleic acid construct, generated recombinantly or synthetically, with nucleic acid elements that are capable of affecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell. Transcription termination signals, enhancers, and other nucleic acid sequences that influence gene expression, can also be included in an expression cassette.

The phrases “heterologous sequence” or a “heterologous nucleic acid”, as used herein, refers to a sequence that originates from a source foreign to the particular host cell, or, if from the same source, is modified from its native form. Thus, a heterologous gene in a eukaryotic host cell includes the modified form of a native gene that is endogenous to the host cell.

The term “isolated” refers to material that is substantially or essentially free from components which interfere with the activity or use of the material. For cells, nucleic acids, and protein of the invention, the term “isolated” refers to material that is substantially or essentially free from components which normally accompany the material as found in its native state. Typically, isolated proteins or nucleic acids of the invention are at least about 80% pure, usually at least about 90%, and preferably at least about 95% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized.

The phrase “operably linked” refers to functional linkage between a nucleic acid expression control sequence (such as a promoter, signal sequence, or array of transcription factor binding sites) and a second nucleic acid sequence, wherein the expression control sequence affects transcription and/or translation of the sequence encoded by the second nucleic acid sequence.

Samples or assays comprising proteases that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a protease is achieved when the activity value relative to the control is about 80%, preferably 50%, more preferably 25-0%. Activation of a protease is achieved when the activity value relative to the control (untreated with activators) is 110%, more preferably 150%, more preferably 200-500% (i.e., two to five fold higher relative to the control), more preferably 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, oligonucleotide, antisense molecule, RNAi molecule, etc., to be tested for the capacity to directly or indirectly modulate protease activity. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons.

“Biological sample” include sections of tissues such as biopsy and autopsy samples, and frozen sections taken for histologic purposes. Such samples include blood, sputum, tissue, cultured cells, e.g., primary cultures, explants, and transformed cells, stool, urine, etc. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

The terms “identical” or percent “identity,” in the context of two or more nucleic acids or amino acid sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or proteins, refers to two or more sequences or subsequences that have at least 60%-70%, preferably 80-85%, most preferably 90-95% nucleotide or amino acid residue identity, when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or proteins are substantially identical is that the protein encoded by the first nucleic acid is immunologically cross reactive with the protein encoded by the second nucleic acid, as described below. Thus, a protein is typically substantially identical to a second protein, for example, where the two peptides differ only by conservative substitutions. Another indication that two nucleic acid sequences are substantially identical is that the two molecules hybridize to each other under stringent conditions, as described below.

The phrase “hybridizing specifically to,” refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a probe will hybridize to its target subsequence, but to no other sequences. Stringent conditions are sequence-dependent and will be different in different circumstances. Longer sequences hybridize specifically at higher temperatures. Generally, stringent conditions are selected to be about 15° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the probes are occupied at equilibrium). Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5× SSC, and 1% SDS, incubating at 42° C., or, 5× SSC, 1% SDS, incubating at 65° C., with wash in 0.2× SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1× SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec −2 min., an annealing phase lasting 30 sec. −2 min., and an extension phase of about 72° C. for 1-2 min. Protocols and guidelines for low and high stringency amplification reactions are provided, e.g., in Innis et al. (1990) PCR Protocols, A Guide to Methods and Applications, Academic Press, Inc. N.Y.).

The term “specific” or “specifically” when used with reference to protein binding or protein cleavage, refers to a binding reaction or proteolytic reaction, respectively, which is determinative of the presence of the protein in the presence of a heterogeneous population of proteins and/or other biomolecules. For example, under designated conditions, a specified protein preferentially binds to a particular protein and does not bind in a significant amount to other proteins present in a sample; or under designated conditions, a specified protease preferentially cleaves a particular protein and does not cleave a significant amount of other proteins in a sample. For example, the μPrAg proteins used in the methods of the present invention comprise a protease cleavage site that is cleaved by a specific cognate extracellular protease.

“Conservatively modified variations” of a particular nucleic acid sequence refers to those nucleotides of the nucleic acid sequence that encode identical or essentially identical amino acid sequences, or where the nucleic acid sequence does not encode an amino acid sequence, to essentially identical sequences. Because of the degeneracy of the genetic code, a large number of functionally identical nucleic acids encode any given protein. For instance, the codons CGU, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every polynucleotide sequence described herein which encodes a protein also describes every possible silent variation, except where otherwise noted. One of skill will recognize that each codon in a nucleic acid (except AUG, which is ordinarily the only codon for methionine, and UGG which is ordinarily the only codon for tryptophan) can be modified to yield a functionally identical molecule by standard techniques. Accordingly, each “silent variation” of a nucleic acid which encodes a protein is implicit in each described sequence.

Furthermore, one of skill will recognize that individual substitutions, deletions or additions which alter, add or delete a single amino acid or a small percentage of amino acids (typically less than 5%, more typically less than 1%) in an encoded sequence are “conservatively modified variations” where the alterations result in the substitution of an amino acid with a chemically similar amino acid. Conservative substitution tables providing functionally similar amino acids are well known in the art.

One of skill will appreciate that many conservative variations of the fusion proteins and nucleic acid which encode the fusion proteins yield essentially identical products. For example, due to the degeneracy of the genetic code, “silent substitutions” (i.e., substitutions of a nucleic acid sequence which do not result in an alteration in an encoded protein) are an implied feature of every nucleic acid sequence which encodes an amino acid. As described herein, sequences are preferably optimized for expression in a particular host cell used to produce the chimeric endonucleases (e.g., yeast, human, and the like). Similarly, “conservative amino acid substitutions,” in one or a few amino acids in an amino acid sequence are substituted with different amino acids with highly similar properties (see, the definitions section, supra), are also readily identified as being highly similar to a particular amino acid sequence, or to a particular nucleic acid sequence which encodes an amino acid. Such conservatively substituted variations of any particular sequence are a feature of the present invention. See also, Creighton (1984) Proteins, W.H. Freeman and Company. In addition, individual substitutions, deletions or additions which alter, add or delete a single amino acid or a small percentage of amino acids in an encoded sequence are also “conservatively modified variations”.

The practice of this invention can involve the construction of recombinant nucleic acids and the expression of genes in transfected host cells. Molecular cloning techniques to achieve these ends are known in the art. A wide variety of cloning and in vitro amplification methods suitable for the construction of recombinant nucleic acids such as expression vectors are well known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1999 Supplement) (Ausubel). Suitable host cells for expression of the recombinant polypeptides are known to those of skill in the art, and include, for example, eukaryotic cells including insect, mammalian and fungal cells (e.g., Aspergillus niger).

Examples of protocols sufficient to direct persons of skill through in vitro amplification methods, including the polymerase chain reaction (PCR) the ligase chain reaction (LCR), Qβ-replicase amplification and other RNA polymerase mediated techniques are found in Berger, Sambrook, and Ausubel, as well as Mullis et al. (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4: 560; and Barringer et al. (1990) Gene 89: 117. Improved methods of cloning in vitro amplified nucleic acids are described in Wallace et al., U.S. Pat. No. 5,426,039.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of antibodies, e.g., recombinant, monoclonal, or polyclonal antibodies, many technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss,-Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (U.S. Pat. No. 4,946,778, U.S. Pat. No. 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Jones et al., Nature 321:522-525 (1986); Riechmann et al., Nature 332:323-327 (1988); Verhoeyen et al., Science 239:1534-1536 (1988) and Presta, Curr. Op. Struct. Biol. 2:593-596 (1992)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

In one embodiment, the antibody is conjugated to an “effector” moiety. The effector moiety can be any number of molecules, including labeling moieties such as radioactive labels or fluorescent labels, or can be a therapeutic moiety. In one aspect the antibody modulates the activity of the protein.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies raised to an LF protein, polymorphic variants, alleles, orthologs, and conservatively modified variants, or splice variants, or portions thereof, can be selected to obtain only those polyclonal antibodies that are specifically immunoreactive with LF proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

By “therapeutically effective dose” or “diagnostically effective dose” herein is meant a dose that produces effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

### DESCRIPTION OF THE PREFERRED EMBODIMENTS

The inventors are the first to appreciate that the specific requirements of PrAg cleavage provide a unique opportunity to modify this protein to make the cleavage dependent on the proteolytic activity of specific extracellular proteases of interest and, therefrom, develop a novel approach for imaging in vivo the activity of these extracellular protease in cells. More specifically, the invention provides highly specific and sensitive methods for imaging the activity of specific extracellular proteases in cells using the anthrax binary toxin system to target cells expressing extracellular proteases with mutant anthrax toxin protective antigens (μPrAg) that bind to receptors on the cells and are cleaved by a specific extracellular protease expressed by the cells, and ligands that specifically bind to the cleaved μPrAg and are linked to a moiety that is detectable by an imaging procedure. The μPrAg proteins used in the methods comprise a protease cleavage site that is cleaved by a specific extracellular protease and is in place of the furin cleavage site of the native PrAg. The methods are useful for diagnosing and treating diseases and undesirable physiological conditions correlated with the activity of extracellular proteases, and for optimizing the therapeutic efficacy of drugs used to treat such diseases and conditions.

A. Construction of Recombinant Proteins Suitable for Use in the Methods for Imaging

The μPrAg proteins and protein ligands suitable for use in the methods of the present invention may be constructed from isolated, known, or cloned proteins by recombinant or synthetic methods described herein or known to one of skill in the art, and may be fusion proteins or mutant, modified, or derivative forms of the isolated, known, or cloned proteins. For example, the μPrAg proteins may be a mutant, modified, or derivative form of the native PrAg in which the furin cleavage site of the native PrAg is replaced with the native cleavage site of a specific extracellular protease, or a modified, mutant, derivative form of the native cleavage site, or an artificial or synthetic cleavage site, that is cleaved by a specific extracellular protease. Also for example, the ligands may be recombinant or synthetic molecules that specifically bind to a μPrAg, or fused to the μPrAg, and capable of being linked to a detectable moiety. The ligand may be linked directly or indirectly to a detectable moiety. For example, the ligand may be linked indirectly to the detectable moiety using a linker as described herein. Further, the ligand may be constructed as a fusion protein comprising the ligand and a μPrAg. In a preferred embodiment, the ligand is a noncytoxic LF or EF. In another preferred embodiment, the ligand is an antibody.

Using known recombinant and/or synthetic methods one skilled in the art can readily obtain or construct nucleic acids that encode the μPrAg proteins and protein ligands suitable for use in the methods of the present invention. For example, nucleic acids that encode known proteins that specifically bind to the PrAg (e.g., lethal factor (LF) and endema factor (EF)) and methods of obtaining such nucleic acids, are known to those of skill in the art. Nucleic acids encoding proteins suitable for use in the methods of the present invention (e.g., cDNA, genomic, or subsequences (probes)) can be cloned, or amplified by in vitro methods such as the polymerase chain reaction (PCR), the ligase chain reaction (LCR), the transcription-based amplification system (TAS), the self-sustained sequence replication system (SSR). A wide variety of cloning and in vitro amplification methodologies are well-known to persons of skill. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864.

DNA that encode a protein suitable for use in the methods of the present invention, for example a μPrAg or ligand that binds thereto, can be prepared by any suitable method described herein or known to those of skill in the art, including for example, cloning and restriction of the appropriate sequences with restriction enzymes. In one preferred embodiment, nucleic acids encoding a protein can be isolated by routine cloning methods. A nucleotide sequence of a known protein as provided in, for example, GenBank or other sequence database (see above) can be used to provide probes that specifically hybridize to the nucleic acid encoding the protein in a genomic DNA, mRNA, or total RNA sample (e.g., in a Southern or Northern blot). Once the target nucleic acid encoding a protein is identified, it can be isolated according to standard methods known to those of skill in the art (see, e.g., Sambrook et al. (1989) Molecular Cloning: A Laboratory Manual, 2nd Ed., Vols. 1-3, Cold Spring Harbor Laboratory; Berger and Kimmel (1987) Methods in Enzymology, Vol. 152: Guide to Molecular Cloning Techniques, San Diego: Academic Press, Inc.; or Ausubel et al. (1987) Current Protocols in Molecular Biology, Greene Publishing and Wiley-Interscience, New York). Further, the isolated nucleic acids can be cleaved with restriction enzymes to create nucleic acids encoding the full-length protein, or subsequences thereof. Using methods known to those of skill in the art, these nucleic acids can then be used to construct the fusion, mutant, or modified proteins suitable for use in the methods of the present invention. For example, the restriction enzyme fragments, encoding a full-length LF or EF, or subsequences thereof, may be ligated to produce a nucleic acid encoding a recombinant protein, including for example a fusion protein, that specifically binds to the μPrAg of the present invention or a modified, mutant, or derivative form that is noncytotoxic. Fusion proteins that comprise a ligand and μPrAg suitable for use in the methods of the present invention are contemplated.

A nucleic acid encoding a protein, or subsequences thereof, can be characterized by assaying for the expressed product, specific binding activity, specific enzymatic activity, or other distinguishing physical or chemical characteristics of the expressed protein using a assays known to those of skill in the art. For example, assays based on the physical, chemical, or immunological properties of the protein can be used to identify the expressed protein or the specific activity of the protein.

Also, a nucleic acid encoding a protein, or subsequences thereof, can be chemically synthesized from the sequences that encodes known proteins. Suitable methods include the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066. Chemical synthesis produces a single stranded oligonucleotide. This can be converted into double stranded DNA by hybridization with a complementary sequence, or by polymerization with a DNA polymerase using the single strand as a template. One of skill would recognize that while chemical synthesis of DNA is often limited to sequences of about 100 bases, longer sequences may be obtained by the ligation of shorter sequences.

Nucleic acids encoding proteins, or subsequences thereof, can be cloned using DNA amplification methods such as polymerase chain reaction (PCR). Thus, for example, the nucleic acid sequence or subsequence is PCR amplified, using a sense primer containing one restriction site (e.g., NdeI) and an antisense primer containing another restriction site (e.g., HindIII). This will produce a nucleic acid encoding the desired protein or subsequence and having terminal restriction sites. This nucleic acid can then be easily ligated into a vector containing a nucleic acid encoding the second molecule and having the appropriate corresponding restriction sites. Suitable PCR primers can be determined by one of skill in the art using the sequence information provided in GenBank or other sources. Appropriate restriction sites can also be added to the nucleic acid encoding the protein or protein subsequence by site-directed mutagenesis. The plasmid containing the protein-encoding nucleotide sequence or subsequence is cleaved with the appropriate restriction endonuclease and then ligated into an appropriate vector for amplification and/or expression according to standard methods. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al., eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science 241: 1077-1080; Van Brunt (1990) Biotechnology 8: 291-294; Wu and Wallace (1989) Gene 4: 560; and Barringer et al. (1990) Gene 89: 117.

Other physical properties of a cloned protein, including a fusion protein, expressed from a particular nucleic acid, can be compared to properties of known proteins to provide another method of identifying suitable sequences or domains of the protein that are determinants of substrate specificity and/or enzymatic activity. In addition, a putative protein gene or recombinant protein gene can be mutated or modified, and the function or activity of the protein, or of particular subsequences or domains of the protein, established by detecting a variation in the function or activity normally produced by the unmutated, unmodified, original, native or control protein.

Functional domains (e.g., binding domains, protease cleavage sites, or translocation domains) of cloned proteins can be identified by using standard methods for mutating or modifying the proteins and testing the modified or mutated proteins for activities such as substrate or binding specificity or enzymatic activity, as described herein. The functional domains of the various proteins can be used to construct nucleic acids encoding recombinant proteins comprising the functional domains of one or more proteins. These fusion proteins can then be tested for the desired substrate or binding specificity or enzymatic activity.

In one approach to cloning suitable recombinant proteins, including fusion proteins, the known nucleic acid or amino acid sequences of cloned proteins are aligned and compared to determine the amount of sequence identity between various proteins. This information can be used to identify and select protein domains that confer or modulate protein activities, e.g., substrate or binding specificity or enzymatic activity based on the amount of sequence identity between the proteins of interest. For example, domains having sequence identity between the proteins of interest, and are associated with a known activity, can be used to construct recombinant proteins containing that domain, and having the activity associated with that domain (e.g., substrate or binding specificity or enzymatic activity).

Production of Antibodies

As mentioned, ligands suitable for use in the methods of the present invention include antibodies that specifically bind to a μPrAg protein. Methods of producing polyclonal and monoclonal antibodies that are immunoreactive to a specific protein (e.g., a μPrAg protein) are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

Immunogens comprising a μPrAg protein, or subsequence thereof, may be used to produce antibodies specifically reactive with the μPrAg protein, or subsequence, respectively. For example, a μPrAg protein, or subsequence thereof, can be cloned, the recombinant protein expressed in eukaryotic or prokaryotic cells, and purified as described herein or by methods known to one of skill in the art. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies may be obtained by various techniques familiar to those skilled in the art. Briefly, spleen cells from an animal immunized with a desired antigen are immortalized, commonly by fusion with a myeloma cell (see, Kohler & Milstein, Eur. J. Immunol. 6:511-519 (1976)). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according to the general protocol outlined by Huse, et al., Science 246:1275-1281 (1989).

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-μPrAg proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular μPrAg ortholog, can also be made, by subtracting out other cross-reacting orthologs from a species such as a non-human mammal. In addition, individual μPrAg proteins can be used to subtract out antibodies that bind both to the receptor and the individual μPrAg proteins.

Once the anti-μPrAg antibodies are available, the μPrAg proteins can be detected by a variety of immunoassay methods. In addition, the antibody can be used therapeutically as a modulator of μPrAg activity. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra.

B. Manipulation of Nucleic Acid Sequences and Amino Acid Sequences

In the embodiments of the μPrAg and ligands suitable for use in the methods of the present invention are manipulated by recombinant and/or synthetic methods to generate proteins with a desired substrate or binding specificity and/or enzymatic activity. One of skill will recognize the many ways of manipulating the nucleic acids encoding a protein, or subsequences thereof to, for example modify or mutate the nucleic acid, to generate the recombinant proteins suitable for use in the methods of the present invention. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. See, e.g., Giliman and Smith (1979) Gene 8:81-97, Roberts et al. (1987) Nature 328: 731-734.

For example, the nucleic acids encoding proteins, or subsequences thereof, can be modified to facilitate the linkage of the two domains to obtain the polynucleotides that encode fusion proteins suitable for use in the methods of the present invention. Protein functional domains that are modified by such methods are also part of the invention. For example, a codon for a cysteine residue can be placed at either end of a domain so that the domain can be linked by, for example, a sulfide linkage. The modification can be done using either recombinant or chemical methods (see, e.g., Pierce Chemical Co. catalog, Rockford Ill.).

The nucleic acids encoding subsequences of a protein, for example a binding domain, protease cleavage site, or translocation domain, can be joined by linker domains, which are typically protein sequences, such as poly-glycine sequences of between about 5 and 200 amino acids, with between about 10-100 amino acids being typical. Proline residues can be incorporated into the linker to prevent the formation of significant secondary structural elements by the linker. Preferred linkers are often flexible amino acid subsequences which are synthesized as part of a recombinant protein. The flexible linker can be an amino acid subsequence comprising a proline such as Gly(x)-Pro-Gly(x) where x is a number between about 3 and about 100. Also, a chemical linker can be used to connect synthetically or recombinantly produced the functional domains of one or more proteins. Such flexible linkers are known to persons of skill in the art. For example, poly(ethylene glycol) linkers are available from Shearwater Polymers, Inc. Huntsville, Ala. These linkers can optionally have amide linkages, sulfhydryl linkages, or heterofunctional linkages.

The recombinant nucleic acids encoding a protein suitable for use in the methods of the present invention may be modified to provide preferred codons which enhance translation of the nucleic acid in a selected cell.

C. Expression Cassettes and Host Cells for Expressing the Recombinant Proteins

The recombinant proteins suitable for use in the methods of the present invention can be expressed in a variety of host cells, including E. coli, other bacterial hosts, yeast, and various higher eukaryotic cells such as the COS, CHO and HeLa cells lines and myeloma cell lines. The host cells can be mammalian cells, plant cells, or microorganisms, such as, for example, yeast cells, bacterial cells, or fungal cells.

Typically, the nucleic acid that encodes the recombinant protein is operably linked to a promoter that is functional in the desired host cell. An wide variety of suitable promoters and vectors are well known, and can be used to express the recombinant proteins suitable for use in the methods. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, expression cassettes into which the nucleic acids that encode fusion proteins are incorporated for high level expression in a desired host cell are useful for expressing the proteins suitable for use in the methods of the present invention.

Expression control sequences that are suitable for use in a particular host cell are often obtained by cloning a gene that is expressed in that cell. Commonly used prokaryotic control sequences, which are defined herein to include promoters for transcription initiation, optionally with an operator, along with ribosome binding site sequences, include such commonly used promoters as the beta-lactamase (penicillinase) and lactose (lac) promoter systems (Change et al., Nature (1977) 198: 1056), the tryptophan (trp) promoter system (Goeddel et al., Nucleic Acids Res. (1980) 8: 4057), the tac promoter (DeBoer, et al., Proc. Natl. Acad. Sci. U.S.A. (1983) 80:21-25); and the lambda-derived P_{L} promoter and N-gene ribosome binding site (Shimatake et al., Nature (1981) 292: 128). The particular promoter system is not critical to the invention, any available promoter that functions in prokaryotes can be used. For expression of fusion proteins in prokaryotic cells other than E. coli, a promoter that functions in the particular prokaryotic species is required. Such promoters can be obtained from genes that have been cloned from the species, or heterologous promoters can be used.

Either constitutive or regulated promoters can be used to express the recombinant proteins. Regulated promoters can be advantageous because the host cells can be grown to high densities before expression of the fusion proteins is induced. High level expression of heterologous proteins slows cell growth in some situations. An inducible promoter is a promoter that directs expression of a gene where the level of expression is alterable by environmental or developmental factors such as, for example, temperature, pH, anaerobic or aerobic conditions, light, transcription factors and chemicals. Such promoters are referred to herein as “inducible” promoters, which allow one to control the timing of expression of the protein or enzyme involved in nucleotide sugar synthesis. For E. coli and other bacterial host cells, inducible promoters are known to those of skill in the art. The promoters and their use are discussed in Sambrook et al., supra. Examples of inducible promoters are numerous and include the arabinose promoter, the lacZ promoter, the metallothionein promoter, and the heat shock promoter, but are not limited thereto.

A construct that includes a polynucleotide of interest operably linked to gene expression control signals that, when placed in an appropriate host cell, drive expression of the polynucleotide is termed an “expression cassette.” Expression cassettes that encode the fusion proteins of the invention are often placed in expression vectors for introduction into the host cell. The vectors typically include, in addition to an expression cassette, a nucleic acid sequence that enables the vector to replicate independently in one or more selected host cells. Generally, this sequence is one that enables the vector to replicate independently of the host chromosomal DNA, and includes origins of replication or autonomously replicating sequences. Such sequences are well known for a variety of bacteria. For instance, the origin of replication from the plasmid pBR322 is suitable for most Gram-negative bacteria. Alternatively, the vector can replicate by becoming integrated into the host cell genomic complement and being replicated as the cell undergoes DNA replication. A preferred expression vector for expression of the enzymes is in bacterial cells is pTGK, which includes a dual tac-gal promoter and is described in PCT Patent Application Publ. NO. WO98/20111.

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems include the lac, tac, and trp operator systems. Other examples of regulatory sequences are those which allow for gene amplification. In eukaryotic systems, these include the dihydrofolate reductase gene which is amplified in the presence of methotrexate, and the metallothionein genes which are amplified with heavy metals. In these cases, the nucleic acid sequence encoding the polypeptide would be operably linked with the regulatory sequence.

The construction of polynucleotide constructs generally requires the use of vectors able to replicate in cells. A plethora of kits are commercially available for the purification of plasmids from bacteria. For their proper use, follow the manufacturer's instructions (see, for example, EasyPrepJ, FlexiPrepJ, both from Pharmacia Biotech; StrataCleanJ, from Stratagene; and, QIAexpress Expression System, Qiagen). The isolated and purified plasmids can then be further manipulated to produce other plasmids, and used to transfect cells. Cloning in Streptomyces or Bacillus is also possible.

Selectable markers are often incorporated into the expression vectors used to express the polynucleotides of the invention. These genes can encode a gene product, such as a protein, necessary for the survival or growth of transformed host cells grown in a selective culture medium. Host cells not transformed with the vector containing the selection gene will not survive in the culture medium. Typical selection genes encode proteins that confer resistance to antibiotics or other toxins, such as ampicillin, neomycin, kanamycin, chloramphenicol, or tetracycline. Alternatively, selectable markers may encode proteins that complement auxotrophic deficiencies or supply critical nutrients not available from complex media, e.g., the gene encoding D-alanine racemase for Bacilli. Often, the vector will have one selectable marker that is functional in, e.g., E. coli, or other cells in which the vector is replicated prior to being introduced into the host cell. A number of selectable markers are known to those of skill in the art and are described for instance in Sambrook et al., supra. A preferred selectable marker for use in bacterial cells is a kanamycin resistance marker (Vieira and Messing, Gene 19: 259 (1982)). Use of kanamycin selection is advantageous over, for example, ampicillin selection because ampicillin is quickly degraded by β-lactamase in culture medium, thus removing selective pressure and allowing the culture to become overgrown with cells that do not contain the vector.

Suitable selectable markers for use in mammalian cells include, for example, the dihydrofolate reductase gene (DHFR), the thymidine kinase gene (TK), or prokaryotic genes conferring drug resistance, gpt (xanthine-guanine phosphoribosyltransferase, which can be selected for with mycophenolic acid; neo (neomycin phosphotransferase), which can be selected for with G418, hygromycin, or puromycin; and DHFR (dihydrofolate reductase), which can be selected for with methotrexate (Mulligan & Berg (1981) Proc. Nat'l. Acad. Sci. USA 78: 2072; Southern & Berg (1982) J. Mol. Appl. Genet. 1: 327).

Selection markers for plant and/or other eukaryotic cells often confer resistance to a biocide or an antibiotic, such as, for example, kanamycin, G 418, bleomycin, hygromycin, or chloramphenicol, or herbicide resistance, such as resistance to chlorsulfuron or Basta. Examples of suitable coding sequences for selectable markers are: the neo gene which codes for the enzyme neomycin phosphotransferase which confers resistance to the antibiotic kanamycin (Beck et al (1982) Gene 19:327); the hyg gene, which codes for the enzyme hygromycin phosphotransferase and confers resistance to the antibiotic hygromycin (Gritz and Davies (1983) Gene 25:179); and the bar gene (EP 242236) that codes for phosphinothricin acetyl transferase which confers resistance to the herbicidal compounds phosphinothricin and bialaphos.

Construction of suitable vectors containing one or more of the above listed components employs standard ligation techniques as described in the references cited above. Isolated plasmids or DNA fragments are cleaved, tailored, and re-ligated in the form desired to generate the plasmids required. To confirm correct sequences in plasmids constructed, the plasmids can be analyzed by standard techniques such as by restriction endonuclease digestion, and/or sequencing according to known methods. Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology, Volume 152, Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1998 Supplement) (Ausubel).

A variety of common vectors suitable for use as starting materials for constructing the expression vectors of the invention are well known in the art. For cloning in bacteria, common vectors include pBR322 derived vectors such as pBLUESCRIP™, and λ-phage derived vectors. In yeast, vectors include Yeast Integrating plasmids (e.g., YIp5) and Yeast Replicating plasmids (the YRp series plasmids) and pGPD-2. Expression in mammalian cells can be achieved using a variety of commonly available plasmids, including pSV2, pBC12BI, and p91023, as well as lytic virus vectors (e.g., vaccinia virus, adeno virus, and baculovirus), episomal virus vectors (e.g., bovine papillomavirus), and retroviral vectors (e.g., murine retroviruses).

The methods for introducing the expression vectors into a chosen host cell are not particularly critical, and such methods are known to those of skill in the art. For example, the expression vectors can be introduced into prokaryotic cells, including E. coli, by calcium chloride transformation, and into eukaryotic cells by calcium phosphate treatment or electroporation. Other transformation methods are also suitable.

Translational coupling may be used to enhance expression. The strategy uses a short upstream open reading frame derived from a highly expressed gene native to the translational system, which is placed downstream of the promoter, and a ribosome binding site followed after a few amino acid codons by a termination codon. Just prior to the termination codon is a second ribosome binding site, and following the termination codon is a start codon for the initiation of translation. The system dissolves secondary structure in the RNA, allowing for the efficient initiation of translation. See Squires, et. al. (1988), J. Biol. Chem. 263: 16297-16302.

The recombinant proteins can be expressed intracellularly, or can be secreted from the cell. Intracellular expression often results in high yields. If necessary, the amount of soluble, active fusion protein may be increased by performing refolding procedures (see, e.g., Sambrook et al., supra.; Marston et al., Bio/Technology (1984) 2: 800; Schoner et al., Bio/Technology (1985) 3:151). In embodiments in which the fusion proteins are secreted from the cell, either into the periplasm or into the extracellular medium, the DNA sequence is linked to a cleavable signal peptide sequence. The signal sequence directs translocation of the fusion protein through the cell membrane. An example of a suitable vector for use in E. coli that contains a promoter-signal sequence unit is pTA1529, which has the E. coli phoA promoter and signal sequence (see, e.g., Sambrook et al., supra.; Oka' et al., Proc. Natl. Acad. Sci. USA (1985) 82: 7212; Talmadge et al., Proc. Natl. Acad. Sci. USA (1980) 77: 3988; Takahara et al., J. Biol. Chem. (1985) 260: 2670).

The recombinant proteins can also be further linked to other bacterial proteins. This approach often results in high yields, because normal prokaryotic control sequences direct transcription and translation. In E. coli, lacZ fusions are often used to express heterologous proteins. Suitable vectors are readily available, such as the pUR, pEX, and pMR100 series (see, e.g., Sambrook et al., supra.). For certain applications, it may be desirable to cleave the non-protein and/or accessory enzyme amino acids from the fusion protein after purification. This can be accomplished by any of several methods known in the art, including cleavage by cyanogen bromide, a protease, or by Factor Xₐ (see, e.g., Sambrook et al., supra.; Itakura et al., Science (1977) 198: 1056; Goeddel et al., Proc. Natl. Acad. Sci. USA (1979) 76: 106; Nagai et al., Nature (1984) 309: 810; Sung et al., Proc. Natl. Acad. Sci. USA (1986) 83: 561). Cleavage sites can be engineered into the gene for the fusion protein at the desired point of cleavage.

More than one fusion protein may be expressed in a single host cell by placing multiple transcriptional cassettes in a single expression vector, or by utilizing different selectable markers for each of the expression vectors which are employed in the cloning strategy.

A suitable system for obtaining recombinant proteins from E. coli which maintains the integrity of their N-termini has been described by Miller et al. Biotechnology 7:698-704 (1989). In this system, the gene of interest is produced as a C-terminal fusion to the first 76 residues of the yeast ubiquitin gene containing a peptidase cleavage site. Cleavage at the junction of the two moieties results in production of a protein having an intact authentic N-terminal reside.

The expression vectors of the invention can be transferred into the chosen host cell by well-known methods such as calcium chloride transformation for E. coli and calcium phosphate treatment or electroporation for mammalian cells. Cells transformed by the plasmids can be selected by resistance to antibiotics conferred by genes contained on the plasmids, such as the amp, gpt, neo and hyg genes.

Fusion proteins that comprise sequences from eukaryotic proteins, may be expressed in, for example, eukaryotic cells, but expression of such proteins are not limited to eukaryotic cells, as described above.

D. Purification of Recombinant Proteins

Once expressed, the recombinant proteins can be purified according to standard procedures of the art, including ammonium sulfate precipitation, affinity columns, column chromatography, gel electrophoresis and the like (see, generally, R. Scopes, Protein Purification, Springer-Verlag, N.Y. (1982), Deutscher, Methods in Enzymology Vol. 182: Guide to Protein Purification., Academic Press, Inc. N.Y. (1990)). Substantially pure compositions of at least about 90 to 95% homogeneity are preferred, and 98 to 99% or more homogeneity are most preferred. Once purified, partially or to homogeneity as desired, the proteins may then be used (e.g., as immunogens for antibody production).

To facilitate purification of the fusion proteins of the invention, the nucleic acids that encode the fusion proteins can also include a coding sequence for an epitope or “tag” for which an affinity binding reagent is available. Examples of suitable epitopes include the myc and V-5 reporter genes; expression vectors useful for recombinant production of fusion proteins having these epitopes are commercially available (e.g., Invitrogen (Carlsbad Calif.) vectors pcDNA3.1/Myc-His and pcDNA3.1/V5-His are suitable for expression in mammalian cells). Additional expression vectors suitable for attaching a tag to the fusion proteins of the invention, and corresponding detection systems are known to those of skill in the art, and several are commercially available (e.g., FLAG” (Kodak, Rochester N.Y.). Another example of a suitable tag is a polyhistidine sequence, which is capable of binding to metal chelate affinity proteins. Typically, six adjacent histidines are used, although one can use more or less than six. Suitable metal chelate affinity proteins that can serve as the binding moiety for a polyhistidine tag include nitrilo-tri-acetic acid (NTA) (Hochuli, E. (1990) “Purification of recombinant proteins with metal chelating adsorbents” In Genetic Engineering: Principles and Methods, J. K. Setlow, Ed., Plenum Press, NY; commercially available from Qiagen (Santa Clarita, Calif.)).

Other haptens that are suitable for use as tags are known to those of skill in the art and are described, for example, in the Handbook of Fluorescent Probes and Research Chemicals (6th Ed., Molecular Probes, Inc., Eugene Oreg.). For example, dinitrophenyl (DNP), digoxigenin, barbiturates (see, e.g., U.S. Pat. No. 5,414,085), and several types of fluorophores are useful as haptens, as are derivatives of these compounds. Kits are commercially available for linking haptens and other moieties to proteins and other molecules. For example, where the hapten includes a thiol, a heterobifunctional linker such as SMCC can be used to attach the tag to lysine residues present on the capture reagent.

One of skill would recognize that modifications can be made to the protein and accessory enzyme enzymatic domains without diminishing their biological activity. Some modifications may be made to facilitate the cloning, expression, or incorporation of the enzymatic domain into a fusion protein. Such modifications are well known to those of skill in the art and include, for example, the addition of codons at either terminus of the polynucleotide that encodes the enzymatic domain to provide, for example, a methionine added at the amino terminus to provide an initiation site, or additional amino acids (e.g., poly His) placed on either terminus to create conveniently located restriction sites or termination codons or purification sequences.

Further, the recombinant protein of the invention can be constructed and expressed as a fusion protein with a molecular “tag” at one end, which facilitates purification of the protein. Such tags can also be used for immobilization of a protein of interest during the glycosylation reaction. Suitable tags include “epitope tags,” which are a protein sequence that is specifically recognized by an antibody. Epitope tags are generally incorporated into fusion proteins to enable the use of a readily available antibody to unambiguously detect or isolate the fusion protein. A “FLAG tag” is a commonly used epitope tag, specifically recognized by a monoclonal anti-FLAG antibody, consisting of the sequence AspTyrLysAspAspAsp AspLys or a substantially identical variant thereof. Other suitable tags are known to those of skill in the art, and include, for example, an affinity tag such as a hexahistidine peptide, which will bind to metal ions such as nickel or cobalt ions.

Further, suitable ligands may be generated from combinatorial libraries by functionally selecting for molecules that specifically bind to a μPrAg protein or region/structure of the μPrAg protein of interest. Suitable μPrAg may be generated from combinatorial libraries by functionally selecting for molecules that are cleaved by a specific extracellular protease. This approach can be used to select for μPrAg with a desired cleavage specificity and activity, e.g., a μPrAg that is cleaved with high specificity and efficiency. See, for example, Abelson (Ed), Meth. Enzymol. 267 Combinatorial Chemistry, Academic Press, San Diego, 1996; Cortese (Ed), Combinatorial Libraries: Synthesis, Screening and Application Potential, Walter de Gruyter, Berlin, 1996; and Wu, Nat. Biotech. 14: 429-431 (1996).

Further, suitable μPrAg or ligands may be isolated and cloned, for example, by direct screening of molecular libraries. For example, phage libraries displaying small peptides could be used for such selection. The selection for may be for example made by simply mixing μPrAg proteins with the phage display library and eluting the phages that bind the μPrAg proteins. If desired, the selection may be performed under “physiological conditions” to eliminate peptides that cross-react with other biomolecules in the cell or cellular organism. Ligands identified in this way may be linked directly to a detectable moiety or indirectly via a linker (e.g., by chemical conjugation or peptide synthesis).

E. Suitable Ligands and μPrAg Proteins and Uses Thereof.

Ligands that are suitable for use in the methods of the present invention are molecules that specifically bind to the cleaved mutant anthrax protective antigen (μPrAg) and are capable of being linked to a detectable moiety. In a preferred embodiment, the ligand is a protein, for example, lethal factor (LF), endema factor (EF), mutated, modified, or derivative forms of LF or EF, e.g., FP59 or LFN, or an LF subsequence that binds to PA comprising up to amino acid 254 of LF, an anti-PA antibody, or other antibody or protein (or mutated, modified, or derivative forms thereof) that bind specifically to the μPrAg used in the methods of the present invention, but is not limited thereto. In another preferred embodiment, the ligand is a noncytotoxic LF or EF. In another embodiment, the ligand is FP59. In another embodiment, the ligand is a subsequence of LF (LFN), located at approximately amino acids 1-254 of LF, which retains the ability to specifically bind PrAg.

The μPrAg proteins suitable for use in the methods of the present invention are recombinant or synthetic proteins that are a modified, mutant, or derivative form of the native PrAg, comprising a cleavage site of a specific extracellular protease in place of the furin cleavage site of the native PrAg and bind a receptor on the surface of a cell expressing an extracellular protease. The protease cleavage site may be a native protease cleavage site, a mutant, modified, or derivative form of a native protease cleavage site, or an artificial or synthetic cleavage site, that is cleaved by a specific extracellular protease. In addition, the mutant, modified, derivative, artificial or synthetic protease cleavag site may confer an activity or specificity that differs from the native protease cleavage site, for example, an increase or decrease in protease cleavage activity or specificity. Further, the μPrAg may be constructed as a fusion protein, for example, a comprising an amino acid sequence encoding a μPrAg and a ligand (e.g., LF or EF), or subsequences thereof.

For example, suitable μPrAg proteins are those which are specifically cleaved by MMPs or plasminogen activators and target MMP- or and plasminogen activators-expressing tumor cells. Such μPrAg proteins can be constructed by replacing the furin cleavage site with a site that is specifically cleaved by MMPs or a plasminogen activator. These μPrAg is specifically cleaved by a specific extracellular protease expressed in a cell, for example in a cancer cell, exposing the ligand binding site, for example the LF binding site. The ligand (e.g., LF) linked to a detectable moiety and/or therapeutic or diagnostic agent then specifically binds to the cleaved μPrAg and the ligand-μPrAg complex is translocated into the cell, thereby specifically targeting and delivering the moiety and/or agent to a specific cell expressing the extracellular protease.

The μPrAg molecules of the invention can be further targeted to a specific cell by making μPrAg fusion proteins. In these mutant fusion proteins, the PrAg receptor binding domain is replaced by a protein such as a growth factor or other cell receptor ligand specifically expressed on the cells of interest. In addition, the PrAg receptor binding domain may be replaced by an antibody that binds to an antigen specifically expressed on the cells of interest.

These proteins provide a way to specifically target tumor cells and image the activity of a specific extracellular protease expressed by the cell without serious damage to normal cells. The methods of the present invention can also be applied to non-cancer inflammatory cells that contain high amounts of cell-surface associated MMPs or plasminogen activators. These μPrAg proteins are thus useful as diagnostic or therapeutic agents.

For example, the close association between MMP and plasminogen activator over expression and tumor metastasis is well demonstrated. The contributions of MMPs in tumor development and metastatic process lead to the development of novel therapies using synthetic inhibitors of MMPs (60, 61, 62). However, these inhibitors only slow growth and do not eradicate the tumors. The methods of the present invention use bacterial toxins modified to target MMPs and plasminogen activators, which are highly expressed and employed by tumor cells for invasion. The μPrAg molecules in which the furin cleavage site is replaced by an MMP or plasminogen activator target site can be used both to deliver compounds such as toxins to the cell, thereby killing the cell, and to deliver a detectable moiety, thereby, providing a means to monitor the therapeutic efficacy of such compounds.

The μPrAg proteins of the invention can also be specifically targeted to cells using μPrAg fusion proteins. In these fusion proteins, the receptor binding domain of PrAg is replaced with a heterologous ligand or molecule such as an antibody that recognizes a specific cell surface protein. PrAg protein has four structurally distinct domains for performing the functions of receptor binding and translocation of the catalytic moieties across endosomal membranes (22). Domain 4 is the receptor-binding domain and has limited contacts with other domains (22). Therefore, PrAg can be specifically targeted to alternate receptors or antigens specifically expressed by tumors by replacing domain 4 with the targeting molecules, such as single-chain antibodies or a cytokines used by other immunotoxins (73). For example, PA-L1 and PA-L2 are directed to alternate receptors, such as GM-CSF receptor, which is highly expressed in leukemias (74) cells and solid tumors including renal, lung, breast and gastrointestinal carcinomas (73-79). It should be highly expected that the combination of these two independent targeting mechanism should allow tumors to be more effectively targeted, and side effects such as hepatotoxicity and vascular leak syndrome should be significantly reduced.

F. Linkers

A linker may serve to link one ligand to one detectable moiety. Alternatively, a linker may link a ligand to more than one detectable moiety. Likewise a detectable moiety may be linked to more than one linker. The use of a plurality of detectable moieties (e.g. several linker-detectable moieties attached to one ligand or several detectable moieties attached to one linker itself attached to one ligand) may enable the detectability of the detectable moiety to be increased (e.g. by increasing its radiopacity, echogenicity or relaxivity) or may enable it to be detected in more than one imaging modality.

Examples of ligands used in the methods of the present invention include, but are not limited to: amino acids, oligopeptides (e.g. hexapeptides), molecular recognition units (MRU's), single chain antibodies (SCA's), proteins, non-peptide organic molecules, Fab fragments, and antibodies that specifically bind the μPrAg of interest. Monoclonal antibodies are preferred over polyclonal antibodies. Preparation of antibodies that react with a desired antigen is well known.

The linker will provide a mono- or multi-molecular skeleton covalently or non-covalently linking a ligand to one or more detectable moieties, e.g. a linear, cyclic, branched or reticulate molecular skeleton, or a molecular aggregate, with in-built or pendant groups which bind covalently or non-covalently, e.g. coordinately, with the ligand and detectable moieties or which encapsulate, entrap or anchor such moieties.

Thus linking of a detectable moiety to a desired ligand may be achieved by covalent or non-covalent means, usually involving interaction with one or more functional groups located on the detectable moiety and/or ligand. Examples of chemically reactive functional groups which may be employed for this purpose include amino, hydroxyl, sulfhydryl, carboxyl, and carbonyl groups, as well as carbohydrate groups, vicinal dials, thioethers, 2-aminoalcohols, 2-aminothiols, guanidinyl, imidazolyl and phenylic groups.

Covalent coupling of a detectable moiety and ligand may therefore be effected using linking agents containing reactive moieties capable of reaction with such functional groups. Examples of reactive moieties capable of reaction with sulfhydryl groups include .alpha.-haloacetyl compounds of the type X—CH.sub.2 CO—(where X=Br, Cl or I), which show particular reactivity for sulfhydryl groups but which can also be used to modify imidazolyl, thioether, phenyl and amino groups as described by Gurd, F. R. N. in Methods Enzymol. (1967) 11, 532. N-Maleimide derivatives are also considered selective towards sulfhydryl groups, but may additionaly be useful in coupling to amino groups under certain conditions. Reagents such as 2-iminothiolane, e.g. as described by Traut, R. et al. in Biochemistry (1973) 12, 3266, which introduce a thiol group through conversion of an amino group, may be considered as sulfhydryl reagents if linking occurs through the formation of disulphide bridges. Thus reagents which introduce reactive disulphide bonds into either the detectable moiety or the ligand may be useful, since linking may be brought about by disulphide exchange between the ligand and detectable moiety; examples of such reagents include Ellman's reagent (DTNB), 4,4′-dithiodipyridine, methyl-3-nitro-2-pyridyl disulphide and methyl-2-pyridyl disulphide (described by Kimura, T. et al. in Analyt. Biochem. (1982) 122, 271).

Examples of reactive moieties capable of reaction with amino groups include alkylating and acylating agents as described by Wong, Y-H. H. in Biochemistry (1979) 24, 5337; Smyth, D. G. et al. in J. Am. Chem. Soc. (1960) 82, 4600 and Biochem. J. (1964) 91, 589; McKenzie, J. A. et al. in J. Protein Chem. (1988) 7, 581; Ross, W. C. J. in Adv. Cancer Res. (1954) 2, 1; Tietze, L. F. in Chem. Ber. (1991) 124, 1215; and Benneche, T. et al. in Eur. J. Med. Chem. (1993) 28, 463.

Representative amino-reactive acylating agents include those described by Schick, A. F. et al. in J. Biol. Chem. (1961) 236, 2477; Herzig, D. J. et al. in Biopolymers (1964) 2, 349; Bodansky, M. et al. in ‘Principles of Peptide Synthesis’ (1984) Springer-Verlag; Wetz, K. et al. in Anal. Biochem. (1974) 58, 347; Rasmussen, J. K. in Reactive Polymers (1991) 16, 199; and Hunter, M. J. and Ludwig, M. L. in J. Am. Chem. Soc. (1962) 84, 3491. Carbonyl groups such as aldehyde functions may be reacted with weak protein bases at a pH such that nucleophilic protein side-chain functions are protonated. Weak bases include 1,2-aminothiols such as those found in N-terminal cysteine residues, which selectively form stable 5-membered thiazolidine rings with aldehyde groups, e.g. as described by Ratner, S. et al. in J. Am. Chem. Soc. (1937) 59, 200. Other weak bases such as phenyl hydrazones may be used, e.g. as described by Heitzman, H. et al. in Proc. Natl. Acad. Sci. USA (1974) 71, 3537.

Aldehydes and ketones may also be reacted with amines to form Schiff's bases, which may advantageously be stabilised through reductive animation. Alkoxylamino moieties readily react with ketones and aldehydes to produce stable alkoxamines, e.g. as described by Webb, R. et al. in Bioconjugate Chem. (1990) 1, 96.

Examples of reactive moieties capable of reaction with carboxyl groups include diazo compounds such as diazoacetate esters and diazoacetamides, which react with high specificity to generate ester groups, e.g. as described by Herriot R. M. in Adv. Protein Chem. (1947) 3, 169. Carboxylic acid modifying reagents such as carbodiimides, which react through O-acylurea formation followed by amide bond formation, may also usefully be employed; linking may be facilitated through addition of an amine or may result in direct ligand-detectable moiety coupling. Useful water soluble carbodiimides include 1-cyclohexyl-3-(2-morpholinyl-4-ethyl)carbodiimide (CMC) and I-ethyl-3-(3-dimethylaminopropyl)carbodiimide (EDC), e.g. as described by Zot, H. G. and Puett, D. in J. Biol. Chem. (1989) 264, 15552. Other useful carboxylic acid modifying reagents include isoxazolium derivatives such as Woodwards reagent K; chlorofommates such as p-nitrophenylchloroformate; carbonyldiimidazoles such as 1,1′-carbonyldiimidazole; and N-carbalkoxydihydroquinolines such as N-(ethoxycarbonyl)-2-ethoxy-1,2-dihydroquinoline.

Other potentially useful reactive moieties include vicinal diones such as p-phenylenediglyoxal, which may be used to react with guanidinyl groups, e.g. as described by Wagner et al. in Nucleic acid Res. (1978) 5, 4065; and diazonium salts, which may undergo electrophilic substitution reactions, e.g. as described by Ishizaka, K. and Ishizaka T. in J. Immunol. (1960) 85, 163. Bis-diazonium compounds are readily prepared by treatment of aryl diamines with sodium nitrite in acidic solutions. It will be appreciated that functional groups in the detectable moiety and/or ligand may if desired be converted to other functional groups prior to reaction, e.g. to confer additional reactivity or selectivity. Examples of methods useful for this purpose include conversion of amines to carboxylic acids using reagents such as dicarboxylic anhydrides; conversion of amines to thiols using reagents such as N-acetylhomocysteine thiolactone, S-acetylmercaptosuccinic anhydride, 2-iminothiolane or thiol-containing succinimidyl derivatives; conversion of thiols to carboxylic acids using reagents such as .alpha.-haloacetates; conversion of thiols to amines using reagents such as ethylenimine or 2-bromoethylamine; conversion of carboxylic acids to amines using reagents such as carbodiimides followed by diamines; and conversion of alcohols to thiols using reagents such as tosyl chloride followed by transesterification with thioacetate and hydrolysis to the thiol with sodium acetate.

Ligand-detectable moiety coupling may also be effected using enzymes as zero-length crosslinking agents; thus, for example, transglutaminase, peroxidase and xanthine oxidase have been used to produce crosslinked products. Reverse proteolysis may also be used for crosslinking through amide bond formation.

Non-covalent ligand-detectable moiety coupling may, for example, be effected by electrostatic charge interactions e.g. between a polylysinyl-functionalised detectable moiety and a polyglutamyl-functionalised ligand, through chelation in the form of stable metal complexes or through high affinity binding interaction such as avidin/biotin binding.

A ligand which comprises or is coupled to a peptide, lipo-oligosaccharide or lipopeptide linker which contains a element capable of mediating membrane insertion may also be useful. One example is described by Leenhouts, J. M. et al. in Febs Letters (1995) 370(3), 189-192.

Coupling may also be effected using avidin or streptavidin, which have four high affinity binding sites for biotin. Avidin may therefore be used to conjugate ligand to detectable moiety if both ligand and detectable moiety are biotinylated. Examples are described by Bayer, E. A. and WIchek, M. in Methods Biochem. Anal. (1980) 26, 1. This method may also be extended to include linking of detectable moiety to detectable moiety, a process which may encourage association of the agent and consequent potentially increased efficacy. Alternatively, avidin or streptavidin may be attached directly to the surface of detectable moiety particles.

Non-covalent coupling may also utilize the bifunctional nature of bispecific immunoglobulins. These molecules can specifically bind two antigens, thus linking them. For example, either bispecific IgG or chemically engineered bispecific F(ab)′.sub.2 fragments may be used as linking agents. Heterobifunctional bispecific antibodies have also been reported for linking two different antigens, e.g. as described by Bode, C. et al. in J. Biol. Chem. (1989) 264, 944 and by Staerz, U. D. et al. in Proc. Natl. Acad. Sci. USA (1986) 83, 1453. Similarly, any detectable moiety and/or ligand containing two or more antigenic determinants (e.g. as described by Chen, Aa et al. in Am. J. Pathol. (1988) 130, 216) may be crosslinked by antibody molecules and lead to formation of cross-linked assemblies of agents of formula I of potentially increased efficacy.

So-called zero-length linking agents, which induce direct covalent joining of two reactive chemical groups without introducing additional linking material (e.g. as in amide bond formation induced using carbodiimides or enzymatically) may, if desired, be used in accordance with the invention, as may agents such as biotin/avidin systems which induce non-covalent reporter-ligand linking and agents which induce electrostatic interactions.

Most commonly, however, the linking agent will comprise two or more reactive moieties, e.g. as described above, connected by a spacer element. The presence of such a spacer permits bifunctional linkers to react with specific functional groups within a molecule or between two different molecules, resulting in a bond between these two components and introducing extrinsic linker-derived material into the detectable moiety-ligand conjugate. The reactive moieties in a linking agent may be the same (homobifunctional agents) or different (heterobifunctional agents or, where several dissimilar reactive moieties are present, heteromultifunctional agents), providing a diversity of potential reagents that may bring about covalent bonding between any chemical species, either intramolecularly or intermolecularly.

The nature of extrinsic material introduced by the linking agent may have a critical bearing on the targeting ability and general stability of the ultimate product. Thus it may be desirable to introduce labile linkages, e.g. containing spacer arms which are biodegradable or chemically sensitive or which incorporate enzymatic cleavage sites. Alternatively the spacer may include polymeric components, e.g. to act as surfactants and enhance the stability of the agent. The spacer may also contain reactive moieties, e.g. as described above to enhance surface crosslinking.

Spacer elements may typically consist of aliphatic chains which effectively separate the reactive moieties of the linker by distances of between 5 and 30 .ANG. They may also comprise macromolecular structures such as poly(ethylene glycols). Such polymeric structures, hereinafter referred to as PEGs, are simple, neutral polyethers which have been given much attention in biotechnical and biomedical applications (see e.g. Milton Harris, J. (ed) “Poly(ethylene glycol) chemistry, biotechnical and biomedical applications” Plenum Press, New York, 1992). PEGs are soluble in most solvents, including water, and are highly hydrated in aqueous environments, with two or three water molecules bound to each ethylene glycol segment; this has the effect of preventing adsorption either of other polymers or of proteins onto PEG-modified surfaces. PEGs are known to be nontoxic and not to harm active proteins or cells, whilst covalently linked PEGs are known to be non-immunogenic and non-antigenic. Furthermore, PEGs may readily be modified and bound to other molecules with only little effect on their chemistry. Their advantageous solubility and biological properties are apparent from the many possible uses of PEGs and copolymers thereof, including block copolymers such as PEG-polyurethanes and PEG-polypropylenes.

Appropriate molecular weights for PEG spacers used in accordance with the invention may, for example, be between 120 Daltons and 20 kDaltons.

The major mechanism for uptake of particles by the cells of the reticuloendothelial system (RES) is opsonisation by plasma proteins in blood; these mark foreign particles which are then taken up by the RES. The biological properties of PEG spacer elements used in accordance with the invention may serve to increase the circulation time of the agent in a similar manner to that observed for PEGylated liposomes (see e.g. Klibanov, A. L. et al. in FEBS Letters (1990) 268, 235-237 and Blume, G. and Cevc, G. in Biochim. Biophys. Acta (1990) 1029, 91-97). Increased coupling efficiency to areas of interest may also be achieved using antibodies bound to the terminii of PEG spacers (see e.g. Maruyama, K. et al. in Biochim. Biophys. Acta (1995) 1234, 74-80 and Hansen, C. B. et al. in Biochim. Biophys. Acta (1995) 1239, 133-144).

Other representative spacer elements include structural-type polysaccharides such as polygalacturonic acid, glycosaminoglycans, heparinoids, cellulose and marine polysaccharides such as alginates, chitosans and carrageenans; storage-type polysaccharides such as starch, glycogen, dextran and aminodextrans; polyamino acids and methyl and ethyl esters thereof, as in homo- and co-polymers of lysine, glutamic acid and aspartic acid; and polypeptides, oligosaccharides and oligonucleotides, which may or may not contain enzyme cleavage sites.

In general, spacer elements may contain cleavable groups such as vicinal glycol, azo, sulfone, ester, thioester or disulphide groups may also be useful; as discussed in, for example, WO-A-9217436 such groups are readily biodegraded in the presence of esterases, e.g. in vivo, but are stable in the absence of such enzymes. They may therefore advantageously be linked to therapeutic agents to permit slow release thereof.

Other potentially useful polymeric spacer materials include those described in Lee, P. I. in Pharm. Res. (1993) 10, 980); San Roman, J. and Guillen-Garcia, P. in Biomaterials (1991) 12, 236-241); Forestier, F., Gerrier, P., Chaumard, C., Quero, A. M., Couvreur, P. and Labarre, C. in J. Antimicrob. Chemoter. (1992) 30, 173-179); Langer, R. in J. Control. Release (1991) 16, 53-60); Finne, U., Hannus, M. and Urtti, A. in Int. J. Pharm. (1992) 78.237-241); Hespe, W., Meier, A. M. and Blankwater, Y. M. in Arzeim.-Forsch./Drug Res. (1977) 27, 1158-1162); Carli, F. in Chim. Ind. (Milan) (1993) 75, 494-9), Imasaki, K., Yoshida, M., Fukuzaki, H., Asano, M., Kumakura, M., Mashimo, T., Yamanaka, H. and Nagai. T. in Int. J. Pharm. (1992) 81, 31-38); Younes, H., Nataf, P. R., Cohn, D., Appelbaum, Y. J., Pizov, G. and Uretzky, G. in Biomater. Artif. Cells Artif. Organs (1988) 16, 705-719); Kobayashi, H., Hyon, S. H. and Ikada, Y. in “Water-curable and biodegradable prepolymers”—J. Biomed. Mater. Res. (1991) 25, 1481-1494); Ratner, B. D., Johnston, A. B. and Lenk, T. J. in J. Biomed. Mater. Res: Applied Biomaterials (1987) 21, 59-90; Sa Da Costa, V. et al. in J. Coll. Interface Sci. (1981) 80, 445-452 and Affrossman, S. et al. in Clinical Materials (1991) 8, 25-31); Song, C. X., Cui, X. M. and Schindler, A. in Med. Biol. Eng. Comput. (1993) 31, S147-150); Bezwada, R. S., Shalaby, S. W. and Newman, H. D. J. in Agricultural and synthetic polymers: Biodegradability and utilization (1990) (ed Glass, J. E. and Swift, G.), 167-174—ACS symposium Series, #433, Washington D.C., U.S.A.—American Chemical Society); Brem, H., Kader, A., Epstein, J. I., Tamargo, R. J., Domb, A., Langer, R. and Leong, K. W. in Sel. Cancer Ther. (1989) 5, 55-65); Tamargo, R. J., Epstein, J. I., Reinhard, C. S., Chasin, M. and Brem, H. in J. Biomed. Mater. Res. (1989) 23, 253-266); Maa, Y. F. and Heller, J. in J. Control. Release (1990) 14, 21-28); and Crommen, J. H., Vandorpe, J. and Schacht, E. H. in J. Control. Release (1993) 24, 167-180).

The following tables list linkers which may be useful for linking a ligand to a detectable moiety

Heterobifunctional linking agents

| Linking agent | Reactivity 1 | Reactivity 2 | Comments |
|---|---|---|---|
| ABH | carbohydrate | photoreactive | |
| ANB-NOS | --NH.sub.2 | photoreactive | |
| APDP(1) | --SH | photoreactive | iodinable |
| | | | disulphide |
| | | | linker |
| APG | --NH.sub.2 | photoreactive | reacts |
| | | | selectively |
| | | | with Arg at pH |
| | | | 7-8 |
| ASIB(1) | --SH | photoreactive | iodinable |
| ASBA(1) | --COOH | photoreactive | iodinable |
| EDC | --NH.sub.2 | --COOH | zero-length |
| | | | linker |
| GMBS | --NH.sub.2 | --SH | |
| sulfo-GMBS | --NH.sub.2 | --SH | water-soluble |
| HSAB | --NH.sub.2 | photoreactive | |
| sulfo-HSAB | --NH.sub.2 | photoreactive | water-soluble |
| MBS | --NH.sub.2 | --SH | |
| sulfo-MBS | --NH.sub.2 | --SH | water-soluble |
| M.sub.2 C.sub.2 H | carbohydrate | --SH | |
| MPBH | carbohydrate | --SH | |
| NHS-ASA(1) | --NH.sub.2 | photoreactive | iodinable |
| sulfo-NHS- | --NH.sub.2 | photoreactive | water-soluble |
| ASA(1) | | | iodinable |
| sulfo-NHS-LC- | --NH.sub.2 | photoreactive | water-soluble, |
| ASA(1) | | | iodinable |
| PDPH | carbohydrate | --SH | disulphide |
| | | | linker |
| PNP-DTP | --NH.sub.2 | photoreactive | |
| SADP | --NH.sub.2 | photoreactive | disulphide |
| | | | linker |
| sulfo-SADP | --NH.sub.2 | photoreactive | water-soluble |
| | | | disulphide |
| | | | linker |
| SAED | --NH.sub.2 | photoreactive | disulphide |
| | | | linker |
| SAND | --NH.sub.2 | photoreactive | water-soluble |
| | | | disulphide |
| | | | linker |
| SANPAH | --NH.sub.2 | photoreactive | |
| sulfo-SANPAH | --NH.sub.2 | photoreactive | water-soluble |
| SASD(1) | --NH.sub.2 | photoreactive | water-soluble |
| | | | iodinable |
| | | | disulphide |
| | | | linker |
| SIAB | --NH.sub.2 | --SH | |
| sulfo-SIAB | --NH.sub.2 | --SH | water-soluble |
| SMCC | --NH.sub.2 | --SH | |
| sulfo-SMCC | --NH.sub.2 | --SH | water-soluble |
| SMPB | --NH.sub.2 | --SH | |
| sulfo-SMPB | --NH.sub.2 | --SH | water-soluble |
| SMPT | --NH.sub.2 | --SH | |
| sulfo-LC-SMPT | --NH.sub.2 | --SH | water-soluble |
| SPDP | --NH.sub.2 | --SH | |
| sulfo-SPDP | --NH.sub.2 | --SH | water-soluble |
| sulfo-LC-SPDP | --NH.sub.2 | --SH | water-soluble |
| sulfo-SAMCA(2) | --NH.sub.2 | photoreactive | |
| sulfo-SAPB | --NH.sub.2 | photoreactive | water-soluble |

Notes: (1) = iodinable; (2) = fluorescent

Homobifunctional linking agents

| Linking agent | Reactivity | Comments |
|---|---|---|
| BS | --NH.sub.2 | |
| BMH | --SH | |
| BASED (1) | photoreactive | iodinable disulphide linker |
| BSCOES | --NH.sub.2 | |
| sulfo-BSCOES | --NH.sub.2 | water-soluble |
| DFDNB | --NH.sub.2 | |
| DMA | --NH.sub.2 | |
| DMP | --NH.sub.2 | |
| DMS | --NH.sub.2 | |
| DPDPB | --SH | disulphide linker |
| DSG | --NH.sub.2 | |
| DSP | --NH.sub.2 | disulphide linker |
| DSS | --NH.sub.2 | |
| DST | --NH.sub.2 | |
| sulfo-DST | --NH.sub.2 | water-soluble |
| DTBP | --NH.sub.2 | disulphide linker |
| DTSSP | --NH.sub.2 | disulphide linker |
| EGS | --NH.sub.2 | |
| sulfo-EGS | --NH.sub.2 | water-soluble |
| SPBP | --NH.sub.2 | |

Homobifunctional linking agents

| Linking agent | Reactivity | Comments |
|---|---|---|
| BS | --NH.sub.2 | |
| BMH | --SH | |
| BASED (1) | photoreactive | iodinable disulphide linker |
| BSCOES | --NH.sub.2 | |
| sulfo-BSCOES | --NH.sub.2 | water-soluble |
| DFDNB | --NH.sub.2 | |
| DMA | --NH.sub.2 | |
| DMP | --NH.sub.2 | |
| DMS | --NH.sub.2 | |
| DPDPB | --SH | disulphide linker |
| DSG | --NH.sub.2 | |
| DSP | --NH.sub.2 | disulphide linker |
| DSS | --NH.sub.2 | |
| DST | --NH.sub.2 | |
| sulfo-DST | --NH.sub.2 | water-soluble |
| DTBP | --NH.sub.2 | disulphide linker |
| DTSSP | --NH.sub.2 | disulphide linker |
| EGS | --NH.sub.2 | |
| sulfo-EGS | --NH.sub.2 | water-soluble |
| SPBP | --NH.sub.2 | |

Agents for protein modification

| Agent | Reactivity | Function |
|---|---|---|
| Ellman's reagent | --SH | quantifies/detects/protects |
| DTT | --S.S-- | reduction |
| 2-mercaptoethanol | --S.S-- | reduction |
| 2-mercaptylamine | --S.S-- | reduction |
| Traut's reagent | --NH.sub.2 | introduces --SH |
| SATA | --NH.sub.2 | introduces protected --SH |
| AMCA-NHS | --NH.sub.2 | fluroescent labelling |
| AMCA-hydrazide | carbohydrate | fluroescent labelling |
| AMCA-HPDP | --S.S-- | fluroescent labelling |
| SBF-chloride | --S.S-- | fluroescent detection of --SH |
| N-ethylmaleimide | --S.S-- | blocks --SH |
| NHS-acetate | --NH.sub.2 | blocks and acetylates --NH.sub.2 |
| citraconic anhydride | --NH.sub.2 | reversibly blocks and |
| | | introduces negative charges |
| DTPA | --NH.sub.2 | introduces chelator |
| BNPS-skatole | tryptophan | cleaves tryptophan residue |
| Bolton-Hunter | --NH.sub.2 | introduces iodinable group |

Preferred linking groups are derived from ligand reactive groups selected from but not limited to:
a group that will react directly with carboxy, aldehyde, amine (NHR), alcohols, sulfhydryl groups, activated methylenes and the like, on the ligand, for example, active halogen containing groups including, for example, chloromethylphenyl groups and chloroacetyl [ClCH.sub.2 C(.dbd.O)—] groups, activated 2-(leaving group substituted)-ethylsulfonyl and ethylcarbonyl groups such as 2-chloroethylsulfonyl and 2-chloroethylcarbonyl; vinylsulfonyl; vinylcarbonyl; epoxy; isocyanato; isothiocyanato; aldehyde; aziridine; succinimidoxycarbonyl; activated acyl groups such as carboxylic acid halides; mixed anhydrides and the like.

A group that can react readily with modified ligand molecules containing a ligand reactive group, i.e., ligands containing a reactive group modified to contain reactive groups such as those mentioned in (1) above, for example, by oxidation of the ligand to an aldehyde or a carboxylic acid, in which case the “linking group” can be derived from reactive groups selected from amino, alkylamino, arylamino, hydrazino, alkylhydrazino, arylhydrazino, carbazido, semicarbazido, thiocarbazido, thiosemicarbazido, sulfhydryl, sulfhydrylalkyl, sulfhydrylaryl, hydroxy, carboxy, carboxyalkyl and carboxyaryl. The alkyl portions of said linking groups can contain from 1 to about 20 carbon atoms. The aryl portions of said linking groups can contain from about 6 to about 20 carbon atoms; and a group that can be linked to the ligand containing a reactive group, or to the modified ligand as noted in (1) and (2) above by use of a crosslinking agent. The residues of certain useful crosslinking agents, such as, for example, homobifunctional and heterobifunctional gelatin hardeners, bisepoxides, and bisisocyanates can become a part of a linking group during the crosslinking reaction. Other useful crosslinking agents, however, can facilitate the crosslinking, for example, as consumable catalysts, and are not present in the final conjugate.

Examples of such crosslinking agents are carbodiimide and carbamoylonium crosslinking agents as disclosed in U.S. Pat. No. 4,421,847 and the ethers of U.S. Pat. No. 4,877,724. With these crosslinking agents, one of the reactants such as the ligand must have a carboxyl group and the other such as a long chain spacer must have a reactive amine, alcohol, or sulfhydryl group. In amide bond formation, the crosslinking agent first reacts selectively with the carboxyl group, then is split out during reaction of the thus “activated” carboxyl group with an amine to form an amide linkage between thus covalently bonding the two moieties. An advantage of this approach is that crosslinking of like molecules, e.g., ligand to ligand is avoided, whereas the reaction of, for example, homo-bifunctional crosslinking agents is nonselective and unwanted crosslinked molecules are obtained.

Preferred useful linking groups are derived from various heterobifunctional cross-linking reagents such as those listed in the Pierce Chemical Company Immunotechnology Catalog—Protein Modification Section, (1995 and 1996).

In addition to the foregoing description, the linking groups, in whole or in part, can also be comprised of and derived from complementary sequences of nucleotides and residues of nucleotides, both naturally occurring and modified, preferably non-self-associating oligonucleotide sequences. Particularly useful, non-limiting reagents for incorporation of modified nucleotide moieties containing reactive functional groups, such as amine and sulfhydryl groups, into an oligonucleotide sequence are commercially available from, for example, Clontech Laboratories Inc. (Palo Alto Calif.) and include Uni-Link AminoModifier (Catalog #5190), Biotin-ON phosphoramidite (Catalog #5191), N-MNT-C6-AminoModifier (Catalog #5202), AminoModifier II (Catalog #5203), DMT-C₆₋₃′Amine-ON (Catalog #5222), C6-ThiolModifier (Catalog #5211), and the like. In one aspect, linking groups of this invention are derived from the reaction of a reactive functional group such as an amine or sulfhydryl group as are available in the above Clontech reagents, one or more of which has been incorporated into an oligonucleotide sequence, with, for example, one or more of the previously described ligand reactive groups such as a heterobifunctional group on the ligand.

By attaching two complementary oligonucleotide sequences one to the ligand and the other to the detectable moiety the resulting double-stranded hybridized oligonucleotide then comprises the linking group between the ligand and detectable moiety.

Other polymer systems that serve as linkers include:
Poly(L or D or DL-amino acids)=proteins and peptides; naturally occuring or synthetic
Pseudo Poly(amino acids)=(amino acids linked by non-amide bonds)
Poly (L or D or DL-lactide) and the co-polymers e.g Poly
(L-lactide/DL-lactide)Poly (glycolide)
L-lactide/glycolide co-polymers
Poly-,-caprolactone and its co-polymers
Polyanhydrides
Poly (ortho esters)
Polyphosphazenes
Long-chain straight or branched lipids (& phospholipids)
Sugars and carbohydrates
Oligonucleotides (see above) as well as mixtures of the above.

Linking agents used in accordance with the invention will in general bring about linking of ligand to detectable moiety or detectable moiety to detectable moiety with some degree of specificity, and may also be used to attach one or more therapeutically active agents.

By way of example, where the detectable moiety is a chelated metal species (e.g. a paramagnetic metal ion or a metal radionuclide), the linker may comprise a chain attached to a metal chelating group, a polymeric chain with a plurality of metal chelating groups pendant from the molecular backbone or incorporated in the molecular backbone, a branched polymer with metal chelating groups at branch termini (e.g. a dendrimeric polychelant). What is required of the linker is simply that it bind the ligand and detectable moieties together for an adequate period. By adequate period is meant a period sufficient for the contrast agent to exert its desired effects, e.g. to enhance contrast in vivo during a diagnostic imaging procedure.

Thus, in certain circumstances, it may be desirable that the linker biodegrade after administration. By selecting an appropriately biodegradable linker it is possible to modify the biodistribution and bioelimination patterns for the ligand and/or detectable moiety. Where ligand and/or detectable moiety are biologically active or are capable of exerting undesired effects if retained after the imaging procedure is over, it may be desirable to design in linker biodegradability which ensures appropriate bioelimination or metabolic breakdown of the ligand and/or detectable moieties. Thus, a linker may contain a biodegradable function which on breakdown yields breakdown products with modified biodistribution patterns which result from the release of the detectable moiety from the ligand or from fragmentation of a macromolecular structure. By way of example for linkers which carry chelated metal ion moieties it is possible to have the linker incorporate a biodegradable function which on breakdown releases an excretable chelate compound containing the detectable moiety. Accordingly, biodegradable functions may if desired be incorporated within the linker structure, preferably at sites which are (a) branching sites, (b) at or near attachment sites for ligands or detectable moieties, or (c) such that biodegradation yields physiologically tolerable or rapidly excretable fragments.

Examples of suitable biodegradable functions include ester, amide, double ester, phosphoester, ether, thioether, guanidyl, acetal and ketal functions.

As discussed above, the linker group may if desired have built into its molecular backbone groups which affect the biodistribution of the contrast agent or which ensure appropriate spatial conformation for the contrast agent, e.g. to allow water access to chelated paramagnetic metal ion moieties. By way of example the linker backbone may consist in part or essentially totally of one or more polyalkylene oxide chains.

The linker may be low, medium or high molecular weight, e.g. up to 2MD. Generally higher molecular weight linkers will be preferred if they are to be loaded with a multiplicity of vectors or detectable moieties or if it is necessary to space the ligand and detectable moiety apart, or if the linker is itself to serve a role in the modification of biodistribution. In general however linkers will be from 100 to 100,000 D, especially 120 D to 20 kD in molecular weight.

Conjugation of linker to ligand and linker to detectable moiety may be by any appropriate chemical conjugation technique, e.g. covalent bonding (for example ester or amide formation), metal chelation or other metal coordinate or ionic bonding, again as described above.

Examples of suitable linker systems include the magnifier polychelant structures of U.S. Pat. No. 5,364,613 and WO90/12050, polyaminoacids (e.g. polylysine), functionalised PEG, polysaccharides, glycosaminoglycans, dendritic polymers such as described in WO93/06868 and by Tomalia et al. in Angew. Chem. Int. Ed. Engl. 29:138-175 (1990), PEG-chelant polymers such as described in W94/08629, WO94/09056 and WO96/26754.

Where the detectable moiety is a chelated metal ion, the linker group will generally incorporate the chelant moiety. Alternatively, the chelated metal may be carried on or in a particulate detectable moiety. In either case, conventional metal chelating groups such as are well known in the fields of radiopharmaceuticals and MRI contrast media may be used, e.g. linear, cyclic and branched polyamino-polycarboxylic acids and phosphorus oxyacid equivalents, and other sulphur and/or nitrogen ligands known in the art, e.g. DTPA, DTPA-BMA, EDTA, DO3A, TMT (see for example U.S. Pat. No. 5,367,080), BAT and analogs (see for example Ohmono et al., J. Med. Chem. 35: 157-162 (1992) and Kung et al. J. Nucl. Med. 25: 326-332 (1984)), the N.sub.2 S.sub.2 chelant ECD of Neurolite, MAG (see Jurisson et al. Chem. Rev. 93:1137-1156 (1993)), HIDA, DOXA (1-oxa-4,7,10-triazacyclododecanetriacetic acid), NOTA (1,4,7-triazacyclononanetriacetic acid), TETA (1,4,8,11-tetraazacyclotetradecanetetraacetic acid), THT 4′-(3-amino-4-methoxy-phenyl)-6,6″-bis(N′,N′-dicarboxymethyl-N-methylhydra zino)-2,2′:6′,2″-terpyridine), etc. In this regard, the reader is referred to the patent literature of Sterling Winthrop, Nycomed (including Nycomed Imaging and Nycomed Salutar), Schering, Mallinckrodt, Bracco and Squibb relating to chelating agents for diagnostic metals, e.g. in MR, X-ray and radiodiagnostic agents. See for example U.S. Pat. No. 4,647,447, EP-A-71564, U.S. Pat. No. 4,687,659, WO89/00557, U.S. Pat. No. 4,885,363, and EP-A-232751.

G. Detectable Moieties

The detectable moieties used in the methods of the present invention may be any moiety capable of detection either directly or indirectly in an imaging procedure described herein or known to one of skill in the art. For example, the following detectable moieties may be used: moieties which emit or may be caused to emit detectable radiation (e.g. by radioactive decay, fluorescence excitation, spin resonance excitation, etc.), moieties which affect local electromagnetic fields (e. paramagnetic, superparamagnetic, ferrimagnetic or ferromagnetic species), moieties which absorb or scatter radiation energy (e.g. chromophores, particles (including gas or liquid containing vesicles), heavy elements and compounds thereof, etc.), and moieties which generate a detectable substance (e.g. gas microbubble generators).

A very wide range of materials detectable by imaging modalities is known from the art and the detectable moiety will be selected according to the imaging modality to be used. Thus for example for ultrasound imaging an echogenic material, or a material capable of generating an echogenic material will normally be selected, for X-ray imaging the detectable moiety will generally be or contain a heavy atom (e.g. of atomic weight 38 or above), for MR imaging the detectable moiety will either be a non zero nuclear spin isotope (such as .sup.19 F) or a material having unpaired electron spins and hence paramagnetic, superparamagnetic, ferrimagnetic or ferromagnetic properties, for light imaging the detectable moiety will be a light scatterer (e.g. a colored or uncolored particle), a light absorber or a light emitter, for magnetometric imaging the detectable moiety will have detectable magnetic properties, for electrical impedance imaging the detectable moiety will affect electrical impedance and for scintigraphy, SPECT, PET etc. the detectable moiety will be a radionuclide.

Examples of suitable detectable moieties are widely known from the diagnostic imaging literature, e.g. magnetic iron oxide particles, gas-containing vesicles, chelated paramagnetic metals (such as Gd, Dy, Mn, Fe etc.). See for example U.S. Pat. No. 4,647,447, WO97/25073, U.S. Pat. No. 4,863,715, U.S. Pat. No. 4,770,183, WO96/09840, WO85/02772, WO92/17212, WO97/29783, EP-A-554213, U.S. Pat. No. 5,228,446, WO91/15243, WO93/05818, WO96/23524, WO96/17628, U.S. Pat. No. 5,387,080, WO95/26205, GB9624918.0.

Particularly preferred detectable moieties are: chelated paramagnetic metal ions such as Gd, Dy, Fe, and Mn, especially when chelated by macrocyclic chelant groups (e.g. tetraazacyclododecane chelants such as DOTA, D03A, HP-DO3A and analogues thereof) or by linker chelant groups such as DTPA, DTPA-BMA, EDTA, DPDP, etc; metal radionuclide such as .sup.90 Y, .sup.99m Tc, .sup.111 In, .sup.47 Sc, .sup.67/Ga, .sup.51 Cr, .sup.177m Sn, .sup.67 Cu, .sup.167 Tm, .sup.97 Ru, .sup.188 Re, .sup.177 Lu, .sup.199 Au, .sup.203 Pb and .sup.141 Ce; superparamagnetic iron oxide crystals; chromophores and fluorophores having absorption and/or emission maxima in the range 300-1400 nm, especially 600 nm to 1200 nm, in particular 650 to 1000 nm; vesicles containing fluorinated gases (i.e. containing materials in the gas phase at 37° C.) which are fluorine containing, e.g. SF.sub.6 or perfluorinated C.sub.1-6 hydrocarbons or other gases and gas precursors listed in WO97/29783); chelated heavy metal cluster ions (e.g. W or Mo polyoxoanions or the sulphur or mixed oxygen/sulphur analogs); covalently bonded non-metal atoms which are either high atomic number (e.g. iodine) or are radioactive, e.g. .sup.123 I, .sup.131 I, etc. atoms; iodinated compound containing vesicles.

Stated generally, the detectable moiety may be (1) a chelatable metal or polyatomic metal-containing ion (ie. TcO, etc), where the metal is a high atomic number metal (e.g. atomic number greater than 37), a paramagentic species (e.g. a transition metal or lanthanide), or a radioactive isotope, (2) a covalently bound non-metal species which is an unpaired electron site (e.g. an oxygen or carbon in a persistent free radical), a high atomic number non-metal, or a radioisotope, (3) a polyatomic cluster or crystal containing high atomic number atoms, displaying cooperative magnetic behavior (e.g. superparamagnetism, ferrimagnetism or ferromagnetism) or containing radionuclides, (4) a gas or a gas precursor (ie. a material or mixture of materials which is gaseous at 37° C.), (5) a chromophore (by which term species which are fluorescent or phosphorescent are included), e.g. an inorganic or organic structure, particularly a complexed metal ion or an organic group having an extensive delocalized electron system, or (6) a structure or group having electrical impedance varying characteristics, e.g. by virtue of an extensive delocalized electron system.

Examples of particular preferred detectable moieties are described in more detail below.

Chelated Metal Moieties

Metal Radionuclides, Paramagnetic Metal Ions, Fluorescent Metal Ions, Heavy Metal Ions and Cluster Ions as described in WO91/14460, WO92/17215, WO96/40287, and WO96/22914; and U.S. Pat. No. 4,647,447, WO89/00557, U.S. Pat. No. 5,367,080, U.S. Pat. No. 5,364,613.

The linker moiety or the particle may contain one or more such chelant groups, if desired metallated by more than one metal species (e.g. so as to provide moieties detectable in different imaging modalities). Particularly where the metal is non-radioactive, it is preferred that a polychelant linker or particulate detectable moiety be used.

A chelant or chelating group as referred to herein may comprise the residue of one or more of a wide variety of chelating agents that can complex a metal ion or a polyatomic ion (e.g. TcO). As is well known, a chelating agent is a compound containing donor atoms that can combine by coordinate bonding with a metal atom to form a cyclic structure called a chelation complex or chelate. This class of compounds is described in the Kirk-Othmer Encyclopedia of Chemical Technology, Vol. 5, 339-368.

The reside of a suitable chelating agent can be selected from polyphosphates, such as sodium tripolyphosphate and hexametaphosphoric acid; aminocarboxylic acids, such as ethylenediaminetetraacetic acid, N-(2-hydroxy)ethylene-diaminetriacetic acid, nitrilotriacetic acid, N,N-di(2-hydroxyethyl)glycine, ethylenebis(hydroxyphenylglycine) and diethylenetriamine pentacetic acid; 1,3-diketones, such as acetylacetone, trifluoroacetylacetone, and thenoyltrifluoroacetone; hydroxycarboxylic acids, such as tartaric acid, citric acid, gluconic acid, and 5-sulfosalicyclic acid; polyamines, such as ethylenediamine, diethylenetriamine, triethylenetetraamine, and triaminotriethylamine; aminoalcohols, such as triethanolamine and N-(2-hydroxyethyl)ethylenediamine; aromatic heterocyclic bases, such as 2,2′-diimidazole, picoline amine, dipicoline amine and 1,10-phenanthroline; phenyls, such as salicylaldehyde, disulfopyrocatechol, and chromotropic acid; aminophenyls, such as 8-hydroxyquinoline and oximesulfonic acid; oximes, such as dimethylglyoxime and salicylaldoxime; peptides containing proximal chelating functionality such as polycysteine, polyhistidine, polyaspartic acid, polyglutamic acid, or combinations of such amino acids; Schiff bases, such as disalicylaldehyde 1,2-propylenediimine; tetrapyrroles, such as tetraphenylporphin and phthalocyanine; sulfur compounds, such as toluenedithiol, meso-2,3-dimercaptosuccinic acid, dimercaptopropanol, thioglycolic acid, potassium ethyl xanthate, sodium diethyldithiocarbamate, dithizone, diethyl dithiophosphoric acid, and thiourea; synthetic macrocyclic compounds, such as dibenzo[18]crown-6, (CH.sub.3).sub.6-[14]-4,11]-diene-N.sub.4, and (2.2.2-cryptate); phosphonic acids, such as nitrilotrimethylene-phosphonic acid, ethylenediaminetetra(methylenephosphonic acid), and hydroxyethylidenediphosphonic acid, or combinations of two or more of the above agents. The residue of a suitable chelating agent preferably comprises a polycarboxylic acid group and preferred examples include: ethylenediamine-N,N,N′,N′-tetraacetic acid (EDTA); N,N,N′,N″,N″-diethylene-triaminepentaacetic acid (DTPA); 1,4,7,10-tetraazacyclododecane-N,N′,N″,N′″-tetraacetic acid (DOTA); 1,4,7,10-tetraazacyclododecane-N,N′,N″-triacetic acid (DO3A); 1-oxa-4,7,10-triazacyclododecane-N,N′,N″-triacetic acid (OTTA); trans(1,2)-cyclohexanodiethylene-triamine-pentaacetic acid (CDTPA).

Other suitable residues of chelating agents comprise proteins modified for the chelation of metals such as technetium and rhenium as described in U.S. Pat. No. 5,078,985, the disclosure of which is hereby incorporated by reference. Suitable residues of chelating agents may also derive from N3S and N2S2 containing compounds, as for example, those disclosed in U.S. Pat. Nos. 4,444,690; 4,670,545; 4,673,562; 4,897,255; 4,965,392; 4,980,147; 4,988,496; 5,021,556 and 5,075,099. Other suitable residues of chelating are described in WO92/08494, the disclosure of which is hereby incorporated by reference.

Representative chelating groups are also described in U.S. Pat. No. 5,559,214 A, WO 95/26754, WO 94/08624, WO 94/09056, WO 94/29333, WO 94/08624, WO 94/08629 A1, WO 94/13327 A1 and WO 94/12216 A1.

Methods for metallating any chelating agents present are within the level of skill in the art. Metals can be incorporated into a chelant moiety by any one of three general methods: direct incorporation, template synthesis and/or transmetallation. Direct incorporation is preferred.

Thus it is desirable that the metal ion be easily complexed to the chelating agent, for example, by merely exposing or mixing an aqueous solution of the chelating agent-containing moiety with a metal salt in an aqueous solution preferably having a pH in the range of about 4 to about 11. The salt can be any salt, but preferably the salt is a water soluble salt of the metal such as a halogen salt, and more preferably such salts are selected so as not to interfere with the binding of the metal ion with the chelating agent. The chelating agent-containing moiety is preferably in aqueous solution at a pH of between about 5 and about 9, more preferably between pH about 6 to about 8. The chelating agent-containing moiety can be mixed with buffer salts such as citrate, acetate, phosphate and borate to produce the optimum pH. Preferably, the buffer salts are selected so as not to interfere with the subsequent binding of the metal ion to the chelating agent.

In diagnostic imaging, a composition comprising the ligand linked to a detectable moiety preferably contains a ratio of metal radionuclide ion to chelating agent that is effective in such diagnostic imaging applications. In preferred embodiments, the mole ratio of metal ion per chelating agent is from about 1:1,000 to about 1:1. In radiotherapeutic applications, the mole ratio of metal ion per chelating agent is preferably from about 1:100 to about 1:1. The radionuclide can be selected, for example, from radioisotopes of Sc, Fe, Pb, Ga, Y, Bi, Mn, Cu, Cr, Zn, Ge, Mo, Ru, Sn, Sr, Sm, Lu, Sb, W, Re, Po, Ta and Ti. Preferred radionuclides include .sup.44 Sc, .sup.64 Cu, .sup.67 Cu, .sup.212 Pb, .sup.68 Ga, .sup.90 Y, .sup.153 Sm, .sup.212 Bi, .sup.186 Re and .sup.188 Re. Of these, especially preferred is .sup.90 Y. These radioisotopes can be atomic or preferably ionic.

The following isotopes or isotope pairs can be used for both imaging and therapy without having to change the radiolabeling methodology or chelator: .sup.47 Sc.sub.21; .sup.141 Ce.sub.58; .sup.188 Re.sub.75; .sup.177 Lu.sub.71; .sup.199 Au.sub.79; .sup.47 Sc.sub.21; .sup.131 I.sub.53; .sup.67 Cu.sub.29; and .sup.123 I.sub.53; .sup.188 Re.sub.75 and .sup.99m Tc.sub.43; .sup.90 Y.sub.39 and .sup.87 Y.sub.39; .sup.47 Sc.sub.21 and .sup.44 Sc.sub.21; .sup.90 Y.sub.39 and .sup.123 I.sub.53; .sup.146 Sm.sub.62 and .sup.Sm.sub.62; and .sup.90 Y.sub.39 and .sup. 111 In.sub.49.

Where the linker moiety contains a single chelant, that chelant may be attached directly to the ligand, e.g. via one of the metal coordinating groups of the chelant which may form an ester, amide, thioester or thioamide bond with an amine, thiol or hydroxyl group on the ligand. Alternatively the ligand and chelant may be directly linked via a functionality attached to the chelant backbone, e.g. a CH.sub.2-phenyl-NCS group attached to a ring carbon of DOTA as proposed by Meares et al. in JACS 110:6266-6267(1988), or indirectly via a homo or hetero-bifunctional linker, e.g. a bis amine, bis epoxide, diol, diacid, difunctionalised PEG, etc. In that event, the bifunctional linker will conveniently provide a chain of 1 to 200, preferably 3 to 30 atoms between ligand and chelant residue.

The chelant moieties within the polychelant linker may be attached via backbone functionalization of the chelant or by utilization of one or more of the metal coordinating groups of the chelant or by amide or ether bond formation between acid chelant and an amine or hydroxyl carrying linker backbone, e.g. as in polylysine-polyDTPA, polylysine-polyDOTA and in the so-called magnifier polychelants, of WO90/12050. Such polychelant linkers may be conjugated to a ligand either directly (e.g. utilizing amine, acid or hydroxyl groups in the polychelant linker) or via a bifunctional linker compound as discussed above for monochelant linkers.

Where the chelated species is carried by a particulate (or molecular aggregate, e.g. vesicular) linker, the chelate may for example be an unattached mono or polychelate (such as Gd DTPA-BMA or Gd HP-DO3A) enclosed within the particle or it may be a mono or polychelate conjugated to the particle either by covalent bonding or by interaction of an anchor group (e.g. a lipophilic group) on the mono/polychelate with the membrane of a vesicle (see for example WO96/11023).

Non-Metal Atomic Moieties

Preferred non-metal atomic moieties include radioisotopes such as .sup.123 I and .sup.131 I as well as non zero nuclear spin atoms such as .sup.18 F, and heavy atoms such as I. Such detectable moieties, preferably a plurality thereof, e.g. 2 to 200, may be covalently bonded to a linker backbone, either directly using conventional chemical synthesis techniques or via a supporting group, e.g. a triiodophenyl group.

In an embodiment of this invention, the use of radioisotopes of iodine is specifically contemplated. For example, if the ligand or linker is comprised of substituents that can be chemically substituted by iodine in a covalent bond forming reaction, such as, for example, substituents containing hydroxyphenyl functionality, such substituents can be labeled by methods well known in the art with a radioisotope of iodine. The iodine species can be used in therapeutic and diagnostic imaging applications. While, at the same time, a metal in a chelating agent on the same ligand-linker can also be used in either therapeutic or diagnostic imaging applications.

As with the metal chelants discussed above, such non-metal atomic moieties may be linked to the linker or carried in or on a particulate linker, e.g. in a vesicle (see WO95/26205 and GB9624918.0).

Linkers of the type described above in connection with the metal moieties may be used for non-metal atomic moieties with the non-metal atomic moiety or groups carrying such detectable moieties taking the place of some or all of the chelant groups.

Organic Chromophoric or Fluorophoric Moieties

Preferred organic chromophoric and fluorophoric moieties include groups having an extensive delocalized electron system, e.g. cyanines, merocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, bis(dithiolene) complexes, bis(benzene-dithiolate) complexes, iodoaniline dyes, bis(S,O-dithiolene) complexes.

Examples of suitable organic or metallated organic chromophores may be found in “Topics in Applied Chemistry: Infrared absorbing dyes” Ed. M. Matsuoka, Plenum, N.Y. 1990, “Topics in Applied Chemistry: The Chemistry and Application of Dyes”, Waring et al., Plenum, N.Y., 1990, “Handbook of Fluorescent Probes and Research Chemicals” Haugland, Molecular Probes Inc, 1996, DE-A-4445065, DE-A-4326466, JP-A-3/228046, Narayanan et al. J. Org. Chem. 60: 2391-2395 (1995), Lipowska et al. Heterocyclic Comm. 1: 427-430 (1995), Fabian et al. Chem. Rev. 92: 1197 (1992), WO96/23525, Strekowska et al. J. Org. Chem. 57: 4578-4580 (1992), WO (Axis) and WO96/17628. Particular examples of chromophores which may be used include xylene cyanole, fluorescein, dansyl, NBD, indocyanine green, DODCI, DTDCI, DOTCI and DDTCI. Particularly preferred are groups which have absorption maxima between 600 and 1000 nm to avoid interference with haemoglobin absorption (e.g. xylene cyanole).

Representative examples of visible dyes include fluorescein derivatives, rhodamine derivatives, coumarins, azo dyes, metalizable dyes, anthraquinone dyes, benzodifuranone dyes, polycyclic aromatic carbonyl dyes, indigoid dyes, polymethine dyes, azacarbocyanine dyes, hemicyanine dyes, barbituates, diazahemicyanine dyes, stryrl dyes, diaryl carbonium dyes, triaryl carbonium dyes, phthalocyanine dyes, quinophthalone dyes, triphenodioxazine dyes, formazan dyes, phenothiazine dyes such as methylene blue, azure A, azure B, and azure C, oxazine dyes, thiazine dyes, naphtholactam dyes, diazahemicyanine dyes, azopyridone dyes, azobenzene dyes, mordant dyes, acid dyes, basic dyes, metallized and premetallized dyes, xanthene dyes, direct dyes, leuco dyes which can be oxidized to produce dyes with hues bathochromically shifted from those of the precursor leuco dyes, and other dyes such as those listed by Waring, D. R. and Hallas, G., in “The Chemistry and Application of Dyes”, Topics in Applied Chemistry, Plenum Press, New York, N.Y., 1990. Additional dyes can be found listed in Haugland, R. P., “Handbook of Fluorescent Probes and Research Chemicals”, Sixth Edition, Molecular Probes, Inc., Eugene Oreg., 1996.

Such chromophores and fluorophores may be covalently linked either directly to the ligand or to or within a linker structure. Once again linkers of the type described above in connection with the metal moieties may be used for organic chromophores or fluorophores with the chromophores/fluorophores taking the place of some or all of the chelant groups.

As with the metal chelants discussed above chromophores/fluorophores may be carried in or on a particulate linker-moieties, e.g. in or on a vesicle or covalently bonded to inert matrix particles that can also function as a light scattering, detectable moiety.

Particulate Moieties or Linker-Moieties

The particulate detectable moieties generally fall into two categories—those where the particle comprises a matrix or shell which carries or contains the detectable moiety and those where the particle matrix is itself the detectable moiety. Examples of the first category are: vesicles (e.g. micelles, liposomes, microballoons and microbubbles) containing a liquid, gas or solid phase which contains the detectable moiety, e.g., an echogenic gas or a precursor therefor (see for example GB 9700699.3), a chelated paramagnetic metal or radionuclide, or a water-soluble iodinated X-ray contrast agent; porous particles loaded with the detectable moiety, e.g. paramagnetic metal loaded molecular sieve particles; and solid particles, e.g. of an inert biotolerable polymer, onto which the detectable moiety is bound or coated, e.g. dye-loaded polymer particles.

Examples of the second category are: light scattering organic or inorganic particles; magnetic particles (ie. superparamagnetic, ferromagnetic or ferrimagnetic particles); and dye particles. Preferred particulate moieties or moiety-linkers include superparamagnetic particles (see U.S. Pat. No. 4,770,183, WO97/25073, WO96/09840, etc.), echogenic vesicles (see WO92/17212, WO97/29783, etc.), iodine-containing vesicles (see WO95/26205 and GB9624918.0), and dye-loaded polymer particles (see WO96/23524).

The particulate moieties may have a ligand attached directly or indirectly to their surfaces. Flow decelerating moieties may be attached to the particles, ie. moieties which have an affinity for the capillary lumen or other organ surfaces which is sufficient to slow the passage of the ligand through the capillaries or the target organ but not sufficient on its own to immobilize the ligand (e.g., see GB9700699.3).

The means by which a particle attachment is achieved will depend on the nature of the particle -surface. For inorganic particles, the linkage to the particle may be for example by way of interaction between a metal binding group (e.g. a phosphate, phosphonate or oligo or polyphosphate group) on the ligand or on a linker attached to the ligand. For organic (e.g. polymeric) particles, ligand attachment may be by way of direct covalent bonding between groups on the particle surface and reactive groups in the ligand, e.g. amide or ester bonding, or by covalent attachment of ligand and particle to a linker. Linkers of the type discussed above in connection with chelated metal moieties may be used although in general the linkers will not be used to couple particles together.

For non-solid particles, e.g. droplets (for example of water insoluble iodinated liquids as described in U.S. Pat. No. 5,318,767, U.S. Pat. No. 5,451,393, U.S. Pat. No. 5,352,459 and U.S. Pat. No. 5,569,448) and vesicles, the linker may conveniently contain hydrophobic “anchor” groups, for example saturated or unsaturated C.sub.12-30 chains, which will penetrate the particle surface and bind ligand to particle. Thus for phospholipid vesicles, the linker may serve to bind the ligand covalently to a phospholipid compatible with the vesicle membrane. Examples of linker binding to vesicles and inorganic particles are described in GB9622368.0 and WO97/25073.

Besides the ligands, other groups may be bound to the particle surface, e.g. stabilizers (to prevent aggregation) and biodistribution modifiers such as PEG. Such groups are discussed for example in WO97/25073, WO96/09840, EP-A-284549 and U.S. Pat. No. 4,904,479.

Preferably the ligands of the present invention are coupled directly or indirectly to a detectable moiety, e.g., with covalently bound iodine radioisotopes, or metal chelates attached directly or via an organic linker group or coupled to a particulate moiety or linker-moiety, e.g. a superparamagnetic crystals (optionally coated, e.g. as in WO97/25073), or a vesicle, e.g. a gas containing or iodinated contrast agent containing micelle, liposome or microballoon.

H. Dosage and Administration of Compositions Containing μPrAg Proteins and Ligands that Binds to the Cleaved μPrAg

The compositions used in the methods of the present invention, comprising a suitable μPrAg and/or ligand linked to a detectable moiety, may be administered to a subject for imaging in amounts sufficient to yield the desired contrast with the particular imaging procedure or modality, e.g., a diagnostically or therapeutically effective amount. The suitable μPrAg proteins and ligands linked to a detectable moiety may be administered together or separately, concurrently or sequentially in a composition.

Examples of imaging modalities suitable for detecting the detectable moiety linked to the ligand include, but are not limited to, magnetic resonance, nuclear magnetic resonance, radioscintigraphy, positron emission tomography, computed tomography, near-infrared fluorescence, X-ray, ultra sound, ultraviolet light, or visible light, wherein the image of the detectable moiety is indicative of the activity of a specific extracellular protease (for example, see Dahnhert, Radiology Review Manual, 4 th Edition, Lippincott, Williams & Wilkins (1999); Brant et al., Fundamentals of Diagnostic Radiobiology, 2 nd Edition, Lippincott, Williams & Wilkins (1999); Weissleder et al., Primer of Diagnostic Imaging, 2 nd Edition, Mosby-Year Book (1997); Buddinger et al., Medical Magnetic Resonance A Primer, Society of Magnetic Resonance, Inc. (1988); and Weissleder et al., Nature Biotech. 17: 375-378 (1999)).

Where the detectable moiety is a metal, generally dosages of from 0.001 to 5.0 mmoles of chelated imaging metal ion per kilogram of patient bodyweight are effective to achieve adequate contrast enhancements. For most MRI applications preferred dosages of imaging metal ion will be in the range of from 0.02 to 1.2 mmoles/kg bodyweight while for X-ray applications dosages of from 0.05 to 2.0 mmoles/kg are generally effective to achieve X-ray attenuation. Preferred dosages for most X-ray applications are from 0.1 to 1.2 mmoles of the lanthanide or heavy metal compound/kg bodyweight. Where the detectable moiety is a radionuclide, dosages of 0.01 to 100 mCi, preferably 0.1 to 50 mCi will normally be sufficient per 70 kg bodyweight. Where the detectable moiety is a superparamagnetic particle, the dosage will normally be 0.5 to 30 mg Fe/kg bodyweight. Where the detectable moiety is a gas or gas generator, e.g. in a microballoon, the dosage will normally be 0.05 to 100 .mu.L gas per 70 kig bodyweight.

The dosage of the compounds of the invention for therapeutic or diagnostic use will depend upon the condition being treated, but in general will be of the order of from 1 pmol/kg to 1 mmol/kg bodyweight. The compounds of the present invention may be formulated with conventional pharmaceutical or veterinary aids, for example emulsifiers, fatty acid esters, gelling agents, stabilizers, antioxidants, osmolality adjusting agents, buffers, pH adjusting agents, etc., and may be in a form suitable for parenteral or enteral administration, for example injection or infusion or administration directly into a body cavity having an external escape duct, for example the gastrointestinal tract, the bladder or the uterus. Thus the compounds of the present invention may be in conventional pharmaceutical administration forms such as tablets, capsules, powders, solutions, suspensions, dispersions, syrups, suppositories etc.

However, solutions, suspensions and dispersions in physiologically or pharmaceutically acceptable carrier media, for example water for injections, will generally be preferred. The compounds according to the invention may therefore be formulated for administration using physiologically acceptable carriers or excipients in a manner fully within the skill of the art. For example, the compounds, optionally with the addition of pharmaceutically acceptable excipients, may be suspended or dissolved in an aqueous medium, with the resulting solution or suspension then being sterilized.

A preferred mode for administering the compositions of the present invention is parenteral, e.g., intravenous administration, in addition to intramuscular, intradermal, and subcutaneous injection. Injection can be systemic or direct injection at the chosen site, e.g., a tumor or specific organ. Parenterally administrable forms, e.g. intravenous solutions, typically are sterile and free from physiologically unacceptable agents, and have low osmolality to minimize irritation or other adverse effects upon administration. Thus, the contrast medium is preferably be isotonic or slightly hypertonic. Suitable vehicles include aqueous vehicles customarily used for administering parenteral solutions such as Sodium Chloride Injection, Ringer's Injection, Dextrose Injection, Dextrose and Sodium Chloride Injection, Lactated Ringer's Injection and other solutions such as are described in Remington's Pharmaceutical Sciences, 15th ed., Easton: Mack Publishing Co., pp. 1405-1412 and 1461-1487 (1975) and The National Formulary XIV, 14th ed. Washington: American Pharmaceutical Association (1975). The solutions can contain preservatives, antimicrobial agents, buffers and antioxidants conventionally used for parenteral solutions, excipients and other additives which are compatible with the chelates and which will not interfere with the manufacture, storage or use of products.

### EXAMPLES

### Example 1

### Delivery of a Compound to Target Cells Using μPrAg Specifically Cleaved by MMP or PA

In this example, mutant PrAg proteins, in which the furin cleavage site of the native PrAg is replaced by an MMP or plasminogen activator cleavage site, and a fusion protein comprising LF and a cytotoxic compound were constructed. The μPrAg proteins bound to receptors on cells expressing the extracellular protease and were specifically cleaved by the extracellular protease translocated by PrAg into the cell.

Two μPrAg, PA-L1 and PA-L2, were constructed in which the furin recognition site is replaced by sequences susceptible to cleavage by MMPs, especially by MMP-2 and MMP-9. When combined with FP59, these two μPrAg proteins specifically killed MMP-expressing tumor cells, such as human fibrosarcoma HT1080 and human melanoma A2058, but did not kill MMP non-expressing cells. Cytotoxicity assay in the co-culture model, in which all the cells were in the same culture environment and were equal accessible to the toxins in the supernatant, showed PA-L1 and PA-L2 specifically killed only MMP-expressing tumor cells HT1080 and A2058, not Vero cells. This result demonstrated activation processing of PA-L1 and PA-L2 mainly occurred on the cell surfaces and mostly contributed by the membrane-associated MMPs, so the cytotoxicity is restricted to MMP-expressing tumor cells. TIMPs are widely present in extracellular milieu and inhibit MMP activity in supernatants. PrAg proteins bind to the cells very quickly with maximum binding happened within 60 min. In contrast to secreted MMPs, membrane-associated MMPs express their proteolytic activities more efficiently by anchoring on cell membrane and enjoying two distinct advantageous properties, which are highly focused on extracellular matrix substrates and more resistant to protease inhibitors present in extracellular milieu.

The data described herein below in Example 1 is also described in Lui et al., Cancer Res. 60: 6061-6067 (2001), incorporated herein by reference.

1.1 Construction of μPrAg with Matrix Metalloprotease Cleavage Sites

Overlap PCR was used to construct the μPrAg proteins with the firin site replaced by MMP substrate octapeptide GPLGMLSQ in PA-L1 and GPLGLWAQ in PA-L2. Wild type PrAg (WT-PA) expression plasmid pYS5 (65) was used as template. We used 5′ primer F (AAAGGAGAACGTATATGA, underlined are SD sequence and start codon of PA) and the phosphorylated primer R1 (pTGAGTTCGAAGATTTTTGTTTTAATTCTGG, annealing to the sequence corresponding to P₁₅₄-S₁₆₃) to amplify the fragment N. We used the mutagenic phosphorylated primer H1 (pGGACCATTAGGAATGTGGAGTCAAAGTACAAGTGCTGGACCTACGGTTCCAG, encoding MMP substrate GPLGMLSQ and S₁₆₈-P₁₇₆) and reverse primer R2 ACGTTTATCTCTTATTAAAAT, annealing to the sequence compassing I₅₈₉-R₅₉₅) to amplify the mutagenic fragment M1. We used a phosphorylated mutagenic primer H2 (pGGACCATTAGGATTATGGGCACAAAGTACAAGTGCTGGACCTACGGTTCCAG, encoding MMP substrate GPLGLWAQ and S₁₆₈-P₁₇₆) to amplify mutagenic fragment M2. Then used primer F and R2 to amplify the ligation products of N and M1, N and M2, respectively, resulting in the mutagenic fragments L1 and L2, in which the coding sequence for furin site (RKKR₁₆₇) were replaced by MMP substrate sequence GPLGMLSQ and GPLGLWAQ, respectively. The HindIII/PstI digests of L1 and L2, which included the mutation sites, were cloned between HindIII and PstI site of pYS5. The resulting expression plasmids were named pYS-PA-L1 and pYS-PA-L2, their expression products, the PrAg mutated proteins, were accordingly named PA-L1 and PA-L2.

1.2 Expression and Purification of WT-PA, PA-L1 and PA-L2

To express WT-PA, PA-L1 and PA-L2, expression plasmids pYS5, pYS-PA-L1 and pYS-PA-L2 were transformed into non-virulent strain B. anthracis UM23C₁₋₁, and grown in FA medium (65) with 20 μg/ml of kanamycin for 16 h at 37° C., PrAg proteins were purified by ammonium sulfate precipitation followed by monoQ column (Pharmacia Biotech) chromatography, as described previously (66).

1.3 In vitro Cleavage of WT-PA, PA-L1 and PA-L2 by Furin MMP-2 and MMP-9

To test whether PA-L1 and PA-L2 had the ability to be processed by MMP-2 and MMP-9 rather than furin, in vitro cleavage of WT-PA, PA-L1 and PA-L2 were performed. For furin cleavage, 50 μl volume of reaction in PBS, pH7.4, 25 mM HEPES, 0.2 mM EDTA, 0.2 mM EGTA, 100 μg/ml ovalbumin, 1.0 mM CaCl₂, 1.0 mM MgCl₂, including 5 μg of WT-PA, PA-L1 and PA-L2, respectively. Digestion was started by addition 0.1 g of soluble form of furin and incubated at 37° C., aliquots (5 μl) were withdrawn at different time points. Cleavage was detected by western blotting with a rabbit anti-PA antibody. For western blotting, the sample aliquots were separated by PAGE using 10-20% gradient Tris-glycine gel (Novex, San Diego, Calif.) and electroblotted to a nitrocellulose membrane (Novex, San Diego, Calif.). The membrane was blocked with 5% (w/v) non-fat milk and hybridized by using rabbit anti-PA polyclonal antibody (#5308). Blot was washed and incubated with an HRP-conjugated goat anti-rabbit antibody (sc-2004, Santa Cruz Biotechnology, Inc., Santa Cruz, Calif.) and was visualized by TMB Stabilized Substrate for HRP (Promega, Madison, Wis.). For MMP-2 and MMP-9 cleavage, 5 μg each of WT-PA, PA-L1 and PA-L2 was incubated with 0.2 μg active MMP-2 or 0.2 μg active MMP-9, respectively, in 50 μl of reactions including 50 mM HEPES, pH7.5, 10 mM CaCl₂, 200 mM NaCl, 0.05% (v/v) Brij-35 and 50 μM ZnSO₄. Aliquots (5 μl) were withdrawn at different time points and were analyzed by western blotting with rabbit anti-PA polyclonal antibody (#5308) as described above.

1.4 Preparation of Cell Extracts and Condition Media for Gelatin Zymography

Cells were cultured in 75 cm² flask to 80-100% of confluence at 37° C. in DMEM supplemented with 10% FCS. Then the cells were washed twice with serum-free DMEM to remove residual FCS, and lysed for 10 min on ice with 1 ml/flask of 0.5% (v/v) Triton X-100 in 0.1 M Tris-HCl, pH 8.0, and scraped with a rubber policeman. The cell lysates were centrifuged at 10,000 rpm for 10 min at 4° C., the concentrations of the proteins were determined by BCA Protein Assay Kit (PIERCE, Rockford, Ill.), and was adjusted to 1 mg/ml by lysis buffer. For collection the conditioned media, the cells were incubated for 24 h with 4 ml/flask of serum-free DMEM. The culture supernatants were harvested, and cellular debris removed by centrifugation at 10,000 rpm for 10 min at 4° C. Cell lysates and conditioned media were frozen at −70° C. or immediately processed for zymographic analysis.

1.5 Gelatin Zymography

Cell extracts (1 ml) or conditioned media normalized to protein concentrations of the corresponding cell extract (3-4 ml) were incubated at 4° C. for 1 h in an end-over-end mixer with 50 μl of gelatin-sepharose 4B (Pharmacia Biotech AB) equilibrated with 50 mM Tris-HCl, 150 mM NaCl, 5 mM CaCl₂, 0.02% (v/v) Tween-20, 10 mM EDTA, pH 7.5. After 4 washes with 1 ml of equilibration buffer containing 200 mM NaCl, the beads were resuspended in 30 μl 4× non-reducing sample buffer, centrifuged to collect the supernatants and loaded on 10% gelatin zymogram gel (Novex, San Diego, Calif.). After electrophoresis, the gel was soaked in Renaturing Buffer (Novex, San Diego, Calif.) for twice with 30 min each to renature gelatinases at room temperature. The gel was then equilibrated in Developing Buffer (Novex, San Diego, Calif.), which added back a divalent metal cation required for enzymatic activity, first for 30 min at room temperature and then in new buffer at 37° C. for overnight. The gel was then stained overnight with 0.5% (w/v) Commassie Brilliant Blue R-250 in 45% (v/v) methanol, 10% acetic acid and destained in the same solution without dye.

1.6 Cytotoxicity Assay with MTT

Cytotoxicity of WT-PA, PA-L1 and PA-L2 to the test cells were performed in 96-well plates. Cells were properly seeded into 96-well plates so that they reached 80 to 100% of confluence the next day. The cells were washed twice with serum-free DMEM to remove residual FCS. Then serially diluted WT-PA, PA-L1 or PA-L2 (from 0 to 1000 ng/ml) combined with FP59 (50 ng/ml) in serum-free DMEM were added to the cells to give a total volume of 200 μl/well. One group of cells was challenged with the toxins for 6 hours, then removed the toxins replaced with fresh DMEM supplemented with 10% FCS. For the cytotoxic action of FP59 relies on inhibition of initial protein synthesis by ADP ribosylating EF-2 and usually need 24-48 hours to show the toxicity, cytotoxicity was allowed to develop for 48 hours. After that cell viability was assayed by adding 50 μl of 2.5 mg/ml MTT (3-[4,5-dimethylthiazol-2-yl]-2,5-diphenyltetrazolium bromide). The cells were incubated with MTT for 45 min at 37° C., live cells oxidized MTT to blue dye precipitated in cytosol while dead cells remained colorless. Then removed media and solubilized the blue precipitate with 100 μl/well of 0.5% (w/v) SDS, 25 mM HCl, in 90% (v/v) isopropanol. The plates were vortexed and the intensity of the oxidized MTT read at 570 nm using the microplate reader. Another group of cells was challenged with the toxins for 48 hours in serum-free DMEM, then viability was determined by cytotoxicity assay with MTT as described above.

1.7 Cytotoxicity Assay in the Co-Culture Model

A co-culture model was used to mimic the in vivo condition to verify whether PA-L1 and PA-L2 specifically targeted and killed MMP expressing tumor cells, not MMP non-expressing cells. Vero, HT1080, A2058 and MDA-MB-231 cells were cultured into the different chambers of 8-chamber slide (Nalge Nunc International, Naperville, Ill.) to 80-100% of confluence. Then the cells were washed twice with serum-free DMEM, the chamber partition was removed, and the slide was put into a petri culture dish with serum free medium, so that the different cells were in the same culture environment. PA, PA-L1 or PA-L2 (300 ng/ml) each plus FP59 (50 ng/ml), or FP59 (50 ng/ml) alone were added to the cells and incubated to 48 hours. Then MTT (0.5 mg/ml) was added for 45 min at 37° C., the partition was remounted, the oxidized MTT was dissolved as described above to determine cell viability for each chamber.

1.8 Cell Binding and Processing Assay of WT-PA, PA-L1 and PA-L2

Binding and processing of WT-PA, PA-L1 and PA-L2 on the surface of Vero cells and HT1080 cells was assayed. Vero and HT1080 cells were grown in 24-well plate to 80-100% of confluence and washed twice with serum-free DMEM to remove residual FCS. Then the cells were incubated with 1000 ng/ml of WT-PA, PA-LL and PA-L2, respectively, for different length of time (0, 10 min, 40 min, 120 min and 360 min) at 37° C. in serum-free DMEM. The cells were washed three times to remove unbound PrAg proteins. Cells were lysed in 100 μl/well modified RIPA lysis buffer (50 mM Tris-HCl, pH 7.4, 1% NP40, 0.25 Na-deoxycholate, 150 mM NaCl, 1 mM EDTA, 1 mM PMSF, 1 mg/ml each of aprotinin, leupeptin and pepstatin) on ice for 10 min. Equal amounts of protein from cell lysates were separated by PAGE using 10-20% gradient Tris-glycine gels (Novex, San Diego, Calif.). After transfer to nitrocellulose membranes, blocking was done with 5% non-fat milk. Western blotting used rabbit anti-PA polyclonal antibody (#5308). Blot was washed and incubated with an HRP-conjugated goat anti-rabbit antibody (sc-2004) (Santa Cruz Biotechnology, Inc., Santa Cruz, Calif.) and was visualized by ECL (PIERCE, Rockford, Ill.).

1.9 Construction and Transfection of MT1-MMP into COS-7 Cells

MT1-MMP cDNA was a generous gift of J. Windsor, UAB. The pEGFPN1 (Clontech Laboratories, Inc., Palo Alto, Calif.) mammalian expression ligand was used for fusing the C-terminus of MT1-MMP to the N-terminus of EGFP (red shifted variant of green fluorescent protein). The MT1-MMP coding sequence was isolated with Tth III and then filled in with Pfu and inserted into the SmaI site of pEGFPN1. COS-7 Cells (2×10⁵ per dish) were transfected with expression ligands (2 μg) by means of SuperFect (10 ml) (Qiagen). Cells were incubated for 3 h. with the DNA-SuperFect complex in the presence of serum and antibiotic containing medium. The complex containing medium was removed and cells grown in fresh serum containing medium for 48 h. Thereafter cells were grown in G418 (Life Technologies, Inc.) containing medium. Cells expressing the MT1-MMP/GFP fusion protein, named COSgMT1, were sorted from non-expressing cells by flow cytometry with a FACstar Plus (Becton Dickinson), excitation at 488 nm.

1.10 Generation of μPrAg that are Specifically Cleaved by MMPs

Crystal structure of PrAg showed that the furin cleavage site RKKR₁₆₇ is in the middle of a surface flexible, solvent exposed loop composed of aa 162 to 175 (22). Cleavage in this loop by furin-like proteases is essential to toxicity. To construct μPrAg proteins specifically processed by MMPs, especially MMP-2 and MMP-9, instead of furin, the furin site RKKR₁₆₇ was replaced by MMP-2 and MMP-9 favorite sequences, GPLGMLSQ and GPLGLWAQ, respectively, resulting in two μPrAg proteins, PA-L1 and PA-L2. These two MMP substrate octapeptides were designed based on the studies of Netzel-Arneet et al (67, 68), in which the sequence specificity of human MMP-2, MMP-9, matrilysin, MMP-1 and MMP-8 had been examined by measuring the rate of hydrolysis of over 50 synthetic oligopeptides. These two octapeptides are favorite substrates of MMP-2 and MMP-9, but also overlap to other MMP species (67, 68). They are also potential substrates for MT1-MMP (69). PA-L1 and PA-L2 coding sequences were constructed by overlap PCR, cloned into E. coli-Bacillus shuttle vector pYS5, and efficiently expressed in non-virulent Bacillus Anthracis UM23C₁₋₁. The expression products were secreted into the culture supernatants and reached to 20 to 50 mg/L. These two mutated PrAg proteins were roughly purified by ammonium sulfate precipitation, followed by mono Q chromatography. The purified mutated PrAg proteins PA-L1 and PA-L2 commiserated with WT-PA in SDS-PAGE, but migrated faster than WT-PA in native gel because of the four positively charged residues RKKR of the furin site were replaced into non-charged MMP octapeptides (data not shown).

To characterize WT-PA and these two μPrAg proteins in susceptibility to proteases, they were subjected to the cleavage with soluble form furin, active form MMP-2 and MMP-9 in vitro. WT-PA was very sensitive to furin, but complete resistant to MMP-2 and MMP-9. In contrast, PA-L1 and PA-L2 were completely resistant to furin, but was efficiently processed into two fragments, PA63 and PA20, by MMP-2 and MMP-9. There was no apparent difference between the two μPrAg proteins in respect to the processing patterns by furin, MMP-2 and MMP-9.

1.11 PA-L1 and PA-L2 Targeted MMP-Expressing Tumor Cells but not MMP Non-Expressing Cells

To test the hypothesis that PA-L1 and PA-L2 only target MMP expressing tumor cells, but not MMP non-expressing normal cells, three human tumor cell lines, fibrosarcoma HT1080, melanoma A2058 and breast cancer MDA-MB-231, and one non-tumor cell line Vero, were employed in cytotoxicity assay. Gelatin zymography showed that HT1080 expressed both MMP-2 and MMP-9, A2058 only expressed MMP-2, MDA-MB-231 only expressed MMP-9, in both conditioned serum-free media and cell extracts, reflecting the gelatinases expressed by these three tumor cell lines were secreted into the media and may also associated with the cell surface. In contrast, Vero cells had very low background of MMP expression.

Targeting and cytotoxicity of WT-PA and the μPrAg proteins to these cells were examined on 96-well plates. When the cells grew to 80 to 100% confluence, different concentrations (from 0 to 1000 ng/ml) of WT-PA, PA-L1 and PA-L2 combined with FP59 (constant at 50 ng/ml) were separately added to the cells and challenged the cells for 6 hours and 48 hours. For the PA-dependent cytotoxicity of FP59 relies on inhibition of initial protein synthesis by ribosylating EF-2, cytotoxicity was allowed to develop for 48 hours. The EC₅₀ (concentration needed to kill half of the cells) of PrAg and the μPrAg proteins were summarized in Table 1. MMP non-expressing Vero cells were quite resistant to PA-L1 and PA-L2, but very sensitive to wild-type PrAG with dose-dependent manner. However, the PA-L1 and PA-L2 nicked by MMP-2 in vitro efficiently killed Vero cells even with 6 hours toxin challenge in dose-dependent manner, demonstrating the non toxicity of PA-L1 and PA-L2 to Vero cells was due to Vero cells lack the ability of cleaving them into the active form PA63. WT-PA, PA-L1 and PA-L2 quickly bound to Vero cells, but only WT-PA could be processed by Vero cells to the active form PA63, while PA-L1 and PA-L2 not. In contrast to Vero cells, the two MMP expressing tumor cells, HT1080, A2058 and MDA-MB-231, were quite susceptible to WT-PA as well as PA-L1 and PA-L2, and the sensitivity to these μPrAg proteins seemed directly correlated with the overall expression levels of MMPs of these tumor cells.

TABLE 1
EC₅₀^{a} (ng/ml) of wild type and mutated PrAg proteins (plus 50 ng/ml
FP59) on target cells

| | Vero | HT1080 | A2058 | MDA-MB-231 | COS-7 | COSgMT1 |
|---|---|---|---|---|---|---|
| WT-PA | 5^{b} (6)^{c} | 2.5 (5.5) | 2 (6) | 1 (2) | 6 (15) | 20 (30) |
| PA-L1 | >>1000 (>>1000) | 2 (10) | 4 (20) | 3 (15) | >>1000 (>>1000) | 20 (40) |
| PA-L2 | >>1000 (>>1000) | 2 (10) | 7 (25) | 4 (30) | >>1000 (>>1000) | 20 (20) |
| Nicked^{d} PA-L1 | 20 | | | | | |
| Nicked^{d} PA-L2 | 20 | | | | | |

^{a}EC₅₀ is the concentration of toxin required to kill half of the cells compared with untreated controls.
^{b}EC₅₀ value for 48 hours toxin treatment
^{c}Value in parenthesis is EC50 for 6 hours toxin treatment
^{d}Nicked by MMP-2

To further demonstrate the cytotoxicity of the μPrAg proteins to the tumor cells was dependent on MMP activity expressed by the target cells, we characterized the effects of the well described MMP inhibitors, BB94 (Batimastat), BB-2516 (Marimastat)), and GM6001, on cytotoxicity of WT-PA, PA-L1 and PA-L2 to HT1080 cells. All these MMP inhibitors, especially GM6001, conferred clear protection to HT1080 cells against the challenge with PA-L1 and PA-L2 plus FP59, but did not protect the cells against WT-PA plus FP59. Thus, targeting and killing of the tumor cells by PA-L1 and PA-L2 was dependent on MMP activity expressed by the target cells.

1.12 PA-L1 and PA-L2 Specifically Targeted MMP-Expressing Tumor Cells in a Co-Culture Model

We designed a co-culture model to mimic the in vivo condition to verify whether PA-L1 and PA-L2 specifically targeted MMP expressing tumor cells, and not MMP non-expressing cells. Vero, HT1080, MDA-MB-231 and A2058 cells were cultured into the different chambers of 8-chamber slides. When the cells reached confluence, the chamber partition was removed and the slide was put into a petri culture dish with serum free medium, so that the different cells were in the same culture environment. PA, PA-L1 or PA-L2 (300 ng/ml) plus FP59 (50 ng/ml), or FP59 (50 ng/ml) alone were separately added to the cells and incubated for 48 hours for cytotoxicity assay as described in Materials and Methods. The result showed WT-PA unselectively targeted and killed all cells, meanwhile PA-L1 and PA-L2 selectively targeted and only killed HT1080, MDA-MB-231 and A2058 cells, but did not harm MMP non-expressing Vero cells. This result defined the relative contributions of membrane-associated versus soluble MMPs, indicated the cleavage of the μPrAg occurred primarily on the surface of the tumor cells instead of in the supernatant. Binding and cleavage of WT-PA, PA-L1 and PA-L2 on the surface of MMP non-expressing Vero cells and MMP expressing HT1080 cells were also directly assessed. Vero and HT1080 cells were incubated with WT-PA, PA-L1 and PA-L2 for 0, 10 min, 40 min, 120 min and 360 min at 37° C., respectively. Then the cells were washed and cell lysates were prepared for western blotting analysis to check the transformation of WT-PA and μPrAg proteins to the active form PA63. The data showed WT-PA, PA-L1 and PA-L2 could be detected in the Vero and HT 1080 cell lysates as soon as 10 min after incubation, demonstrating WT-PA and μPrAg proteins could quickly bound to the cell surface. WT-PA was cleaved by both of these two cell lines. In contrast, PA-L1 and PA-L2 were only processed by MMP expressing HT1080 cells but not MMP non-expressing Vero cells, consistent with the previous results that PA-L1 and PA-L2 could only be cleaved by MMPs and selectively killed MMP-expressing tumor cells. HT1080 cells cleaved WT-PA, PA-L1 and PA-L2, but the results showed the cells cleaved WT-PA more efficiently than PA-L1 and PA-L2, reflecting the activity of furin or furin-like proteases was higher than that of MMPs on the cell surface. We also analyzed the processing status of PA-L1 and PA-L2 in the culture supernatants of HT1080 cells, and could not detect their active form PA63 in the overnight culture supernatants, but with time increasing the randomly breakdown products showed up.

1.13 MT1-MMP Played a Role in Activation of PA-L1 and PA-L2

Zymographic analysis showed COS-7 cells expressed very negligible amount gelatinases. Thus, just as expected, COS-7 cells were resistant to PA-L1 and PA-L2 plus FP59, but susceptible to WT-PA plus FP59. To examine the role of MT1-MMP in activation of PA-L1 and PA-L2, encoding sequence of MT1-MMP was transfected into COS-1 cells, resulting in a stable transfectant COSgMTI in which expression of MT1-MMP was detected by western blotting. In contrast to COS-7 cells, COSgMTI became very sensitive to PA-L1 and PA-L2, indicating MT1-MMP played a role in activation of these μPrAg proteins, either by directly processing the cell bound μPrAg proteins, or by indirect way that activated pro-MMP-2 or other MMPs first, which in turn processed μPrAg proteins to their active form PA₆₃. It seemed unlikely the later one, for COS-7 cells expressed negligible amount of MMPs.

### Example 2

### Construction of μPrAg with Matrix Metalloprotease Cleavage Sites

The μPrAg proteins were constructed and tested as described in Example I, substituting one of the following plasminogen activator cleavage sites of Table 2 for the MMP cleavage sites described above. Phage display libraries were used to identify sequences having specificity for a particular protease (see, e.g., Coombs et al., J. Biol. Chem. 273:4323-4328 (1998); Ke et al., J. Biol. Chem. 272:20456-20462 (1997); Ke et al., J. Biol. Chem. 272:16603-16609 (1997)). These libraries can be used by one of skill in the art to select sequences specifically recognized by MMP and plasminogen activator proteases.

TABLE 2
uPA and tPA cleavage sites

| | uPA | tPA | uPA:tPA |
|---|---|---|---|
| Substrate sequence | Kcat/Km | Kcat/Km | selectivity |
| PCPGRVVGG | 0.88 | 0.29 | 3.0 |
| PGSGRSA | 1200 | 60 | 20 |
| PGSGKSA | 193 | 1.6 | 121 |
| PQRGRSA | 45 | 850 | 0.005 |

### Example 3

### Imaging of the Activity of the Protease Urokinase Plasminogen Activator (uPA) in Tumor Cells Using μPrAg that that is Specifically Cleaved by uPA

The data referred to herein below in Example 3 is described in Lui et al., J. Biol. Chem. 276: 17976-17984 (2001), incorporated herein by reference.

3.1 Introduction

Urokinase plasminogen activator receptor (uPAR) binds pro-urokinase plasminogen activator (pro-uPA) and thereby localizes it near plasminogen, causing the generation of active uPA and plasmin on the cell surface. uPAR and uPA are overexpressed in a variety of human tumors and tumor cell lines, and expression of uPAR and uPA is highly correlated to tumor invasion and metastasis. In this example, mutant anthrax toxin protective antigen (PrAg) proteins were constructed in which the furin cleavage site is replaced by sequences cleaved specifically by uPA. These μPrAg proteins were specifically cleaved on the surface of uPAR-expressing tumor cells in the presence of pro-uPA and plasminogen. The cleaved μPrAg proteins were then specifically bound by fusion protein comprising anthrax toxin lethal factor residues 1-254 fused to the ADP-ribosylation domain of Pseudomonas exotoxin A. The μPrAg-fusion protein complex was then translocated into the cell, thereby, killing the uPAR-expressing tumor cells. The specific cleavage and target specificity of these μPrAg proteins was dependent on the integrity of the tumor cell surface-associated plasminogen activation system and, thus, reflect the activity of specific extracellular proteases associated with tumor cells expressing the proteases.

Also constructed were μPrAg proteins that specifically targeted tissue plasminogen activator-expressing cells which may be useful as new therapeutic agents for cancer treatment.

3.2 Construction of a Mutant Anthrax Toxin Protective Antigen (μPrAg) Specifically Cleaved by uPA

A modified overlap PCR method was used to construct the μPrAg proteins in which the firin site of the native PrAg was replaced by: 1) the plasminogen-derived sequence PCPGRVVGG in the clone PrAg-U1; 2) the preferred uPA substrate sequences PGSGRSA and PGSGKSA in the clone PrAg-U2 and PrAg-U3, respectively; and 3) the preferred tPA sequence PQRGRSA in the clone PrAg-U4 (Table 2). Plasmid pYS5 (62) was used as both PCR template and expression vector. The native Pfu DNA polymerase (Stratagene, La Jolla, Calif.) was used in the PCR reactions.

The following primers were used in the specified amplification reactions:

The 5′-primer F, AAAGGAGAACGTATATGA (Shine-Dalgarno and start codons are underlined), and the phosphorylated reverse primer R1, pTGGTGAGTTCGAAGATTTTTGTTTTAATTCTGG (the first three nucleotides encodes P, the others anneal to the sequence corresponding to P¹⁵⁴ to S¹⁶³), was used to amplify a fragment designated “N.” The mutagenic phosphorylated primer Hi, pTGTCCAGGAAGAGTAGTTGGAGGAAGTACAAGTGCTGGACCTACGGTTCCAG, encoding CPGRVVGG and S¹⁶⁸ to P¹⁷⁶ and reverse primer R2, ACGTTI′ATCTCTTATTAAA.AT, annealing to the sequence encoding I⁵⁸⁹ to R⁵⁹⁵, was used to amplify a mutagenic fragment ‘M1.” The phosphorylated mutagenic primer H2, pGGAAGTGGAAGATCAGCA.AGTACAAGTGCTGACCTACGCI′1I2CCAG, encoding GSGRSA and S¹⁶⁸ to P176, and reverse primer R2, was used to amplify a mutagenic fragment “M2.” The phosphorylated mutagenic primer H3, pGGAAGTGGAAAATCAGCAAGTACAAGTGCTGGACCTACGGTTCCAG, encoding GSGKSA and S¹⁶⁸ to P¹⁷⁶, and reverse primer P.2, was used to amplify a mutagenic fragment “M3.” The phosphorylated mutagenic primer H4, pCAGAGAGGAAGATCAGCAAGTACAAGTGCTGGACCTACGGTTCCAG, encoding QRGRSA and S¹⁶⁸ to P¹⁷⁶ and reverse primer R2, was used to amplify a mutagenic fragment ‘M4.”

The primers F and R2 were used to amplify the ligated products of N+M1, N+M2, N+M3, and N+M4, respectively, resulting in the mutagenized fragments U1, U2, U3, and U4 in which the coding sequence for the furin cleavage site of the native PrAg (¹⁶⁴RKKR¹⁶⁷) is replaced by the cleavage sites of uPA or tPA. The 670-bp HindIII/PstI fragments from the digests of U1, U2, U3, and U4 were cloned between the HindIII and PstI sites of pYS5. The resulting μPrAg proteins were accordingly named PrAg-U1, PrAg-U2, PrAg-U3, and PrAg-U4.

Also, as a control, a μPrAg named PrAg-U7, in which the sequence ¹⁶⁴RKKR¹⁶⁷ (i.e., the furin cleavage site of the native PrAg) is replaced by the sequence PGG. This protein is predicted to be resistant to all cell surface proteases. DNA sequencing analyses confirmed the sequences of the mutant PrAg constructs.

3.3 Expression and Purification of μPrAg Proteins

Plasmids encoding the constructs described above were transformed into the non-virulent strain Bacillus anthracis UM23C₁₋₁, and transformants were grown in FA medium (62) with 20 μg/ml kanamycin for i6 h at 37° C. The μPrAg proteins were concentrated from the culture supernatants and purified by chromatography on a MonoQ column (Amersham Pharmacia Biotech, Piscataway, N.J.) by the methods described previously (63).

3.4 In vitro Cleavage of PrAg Proteins by uPA, tPA, and Furin

Reaction mixtures of 50 μl containing 5 μg of the PrAg and μPrAg proteins were incubated at 37° C. with 5 μl of soluble furin or 0.5 μg of uPA or tPA. Furin cleavage was performed as described above or previously (55). Cleavage with uPA or tPA was performed in 150 mM NaCl, 10 mM Tris-HCl (pH 7.5). Aliquots withdrawn at intervals were analyzed by SDS-polyacrylamide gel electrophoresis (PAGE) using 4-20% gradient Tris-glycine gels (Novex, San Diego, Calif.), and proteins were either visualized by Commassie Blue staining or were electroblotted to a nitrocellulose membrane (Novex). Membranes were blocked with 5% (wfv) non-fat milk, incubated sequentially with rabbit anti-PrAg polyclonal antibody (no. 5308) and horseradish peroxidase-conjugated goat anti-rabbit antibody (sc-2004, Santa Cruz Biotechnology, Inc., Santa Cruz, Calif.), and visualized by detection of horseradish peroxidase by SuperSignal West Pica chemiluminescent substrate (Pierce, Rockford, Ill.). To verify the cleavage sites, digestions of native PrAg by furin, PrAg-U2 and-U3 by uPA, and PrAg-U4 by tPA (Calbiochem) were performed for 3 h at 37° C. as described above. Then the resulting PrAg63s were separated by SDS.NuPAGE electrophoresis (Novex), and the proteins were transferred onto Imniobilon-P polyvinylidene difluoride membranes (Millipore, Bedford, Mass.) and visualized by Coomassie Blue staining. The protein bands were cut out and sequenced by the Protein and Nucleic Acid Laboratory, Center for Biologics Evaluation and Research, FDA using an ABI model 494A protein sequencer.

3.5 Cytotoxicity Assays with MTT

Cells were cultured in 96-well plates to ˜50% confluence and washed twice with serum-free DMEM to remove residual serum. Then the cells were preincubated for 30 minutes with serum-free DMEM containing 100 μg/ml pro-uPA and 1 μg/ml Gluplasminogen with or without PAI-1, aprotinin, α₂-antiplasmin, ATF, or the uPAR blocking antibody R3. PrAg proteins (0-1000 ng/ml) combined with FP59 (50 ng/ml) were added to the cells to give a total volume of 200 μl/well. Cells were incubated with the toxins for 6 h, after which the medium was replaced with fresh DMEM supplemented with 10% fetal calf serum. Cell viability was assayed by adding 50 μl of 2.5 mg/ml MTT (3-[4,5-dimethylthiazol-2-yl]-2,5-diphenyltetrazolium bromide) at 48 h. The cells were incubated with MTI′ for 45 min at 37° C., the medium was removed, and the blue pigment produced by viable cells was dissolved in 100 μl of 0.5% (w/v) SDS, 25 mM HCl, in 90% (v/v) isopropanol. The plates were vortexed and the oxidized MTT was measured as A₅₇₀ using a microplate reader.

3.6 Binding and Processing of Pro-PA and PrAg-U2 by Cultured Cells

Cells were cultured in 24-well plates to confluence, washed, and incubated in serum-free DMEM with 1 μg/ml pro-uPA, 1 μg/ml PrAg-U2, and 1 μg/ml Gluplasminogen, and 2 mg/ml bovine serum albumin (BSA) at 37° C. for various lengths of times. The cells were washed five times to remove unbound pro-uPA and PrAg-U2. When PAI-1 was tested, it was incubated with cells for 30 min prior to the addition of pro-uPA and PrAg-U2. When tranexamic acid was tested, cells were preincubated with serum-free DMEM containing 2 mg/ml BSA, 1 mM tranexamic acid, without plasminogen, for 30 mm before the addition of pro-uPA and PrAg-U2. Cells were lysed in 100 μl/well of modified radioimmune precipitation lysis buffer (50 mM Tris-HCl, pH 7.4, 1% Nonidet P.40, 0.25% sodium deoxycholate, 150 mM NaCl, 1 mM EDTA, 1 mM phenylmethylsulfonyl fluoride, 1 μg/ml each of aprotinin, leupeptin, and pepstatin) on ice for 10 min. Equal amounts of protein from cell lysates and equal volumes of the conditioned media were separated by PAGE using 4—20% gradient Tris-glycine gels (Novex). Western blotting was performed as described above to detect pro-uPA and PrAg-U2 and their cleavage products by using the monoclonal antibody against human uPA beta chain (no. 394) and anti-PrAg polyclonal antibody (no. 5308).

3.7 Cytotoxicity Assay in a Co-Culture System

A co-culture model like that described previously (55) was employed to determine whether PrAg-U2 killed uPAR-overexpressing tumor cells without affecting by-stander, uPAR non-expressing cells. Briefly, HeLa and 293 cells were co-cultured in separate compartments of eight-chamber slides. With the partitions removed, the culture slides were incubated for 6 h with native PrAg or PrAg-U2 (each 300 ng/ml) combined with FPS9 (50 ng/ml) in serum-free DMEM containing 100 ng/ml pro-uPA and 1 μg/ml Glu-plasminogen. After 48 h, the partitions were replaced and MTT-containing medium was added to each chamber to assess cell viability, as described above.

3.8 Imaging of a Specific Extracellular Protease Activity in the Tissue of Mice Administered a Composition Containing μPrAg and a Ligand

Wild-type mice designated as C57BL/6J, uPA-deficient mice designated as C57BL/6J-uPA, uPAR-deficient mice designated as C57BL/6J-uPAR, and plasminogen-deficient mice designated as C57BL/6J-Plg were administered, by intraperitoneal injection, either of the following compositions: 1) PBS; or 2) 200 μg PrAg-U2 (Protective Antigen with the furin site replaced by a uPA cleavage site) and 10 μg FP59 (LF residues 1-254 fused to the ADP-ribosylating domain of Pseudomonas exotoxin A), in PBS.

Twenty eight hours after administration of the above compositions, the mice were euthanized and tissues of the euthanized mice were fixed in 4% paraformaldehyde, processed into paraffin, sectioned and the activity of the uPA image by staining with hematoxylin and eosin dyes.

The resulting high magnification photomicrograph images show multifocal lymphocytolysis was present in all parts of the lymphatic system that were examined (white pulp of the spleen, gastric-associated lymphoid tissue, mesenteric lymph nodes, and thymus) of C57BL/6J mice injected with PrAg-U2 and FP59, but not C57BL/6J mice injected with PBS or PrAg-U2. Necrotic, pyknotic, or karyohectic cells were also identified in the red pulp of the spleen, bone marrow, bone, U2, but not C57BL/6J mice injected with PBS, or C7BL/6J-μPA mice, C57BL/6J-μPAR mice, or C57BL/6J-Plg mice injected with either PBS or PrAg-U2.

Materials and Methods

Reagents

FP59 and a soluble form of furin were prepared as described previously (61). Rabbit anti-PrAg polyclonal antiserum (serum no. 5308) was made in our laboratory. Reagents obtained from American Diagnostica Inc. (Greenwich, Conn.) included pro-uPA (single-chain uPA, no. 107), uPA (no. 124), tPA (no. 116), human urokinase amino-terminal fragment (ATF, no. 146), human Gluplasminogen (no. 410), human PAI-1 (no. 1094), α₂-antiplasmin (no. 4030), monoclonal antibody against human uPA B-chain (no. 394), and goat polyclonal antibody against human t-PA (no. 387). tPA not containing protein stabilizer was purchased from Calbiochem (San Diego, Calif.). Aprotinin and tranexamic acid were purchased from Sigma Chemical Co. (St. Louis, Mo.). The uPAR monoclonal antibody P.3 was a gift from Dr. Gunilla Heyer Hansen (Finsen Laboratory, Copenhagen, Denmark).

Cells and Culture Medium

Human 293 kidney cells, human cervix adenocarcinoma HeLa cells, human melanoma A2058 cells, and human melanoma Bowes cells were obtained from American Type Culture Collection (Manassas, Va.). Mouse Lewis lung carcinoma cell line LL3 was kindly provided by Dr. Michael S. O'Reilly (Boston, Mass.). These cells were grown in Dulbecco's modified Eagle's medium (DMEM) with 0.45% glucose, 10% fetal bovine serum (FCS), 2 mM glutamine, and 50 μg/ml gentamicin. Human umbilical vein endothelial cells (HUVEC) were obtained from Clonetics Corp. (Walkersville, Md.) and were grown in RPMI 1640 containing 20% defined and supplemented bovine calf serum (HyClone Laboratories, Inc., Logan, Utah), 5 units/ml heparin (Fisher Scientific, Pittsburgh, Pa.), 100 units/ml penicillin, and 0.2 mg/ml endothelial cell growth supplement (Collaborative Research), 100 μg/ml streptomycin, 50 μg/ml gentamicin, and 2.5 μg/ml amphotericin B (Life Technologies, Rockville, Md.). Cells were maintained at 37° C. in a 5% CO₂ environment.

Results

Directing uPA- and tPA-specific Proteolysis to Anthrax PrAg

The furin cleavage site, ¹⁶⁴RKKR¹⁶⁷, is located in a surface-exposed, flexible loop of PrAg composed of residues 162-175 (64). μPrAg proteins were constructed in which this sequence is replaced by sequences that are preferred uPA or tPA substrates (Table 2). The mutant PrAg protein PrAg-U1 contains the sequence PCPGRVVGG, corresponding to positions P5 to P4′ in the physiological substrate plasminogen. Protein PrAg-U2 contains the sequence PGSGRSA, which includes the consensus sequence SGRSA, recently identified as the minimized optimum substrate for uPA (59). Because the sequence SGRSA is cleaved by uPA 1363-fold times more efficiently than the physiological cleavage site present in plasminogen, and because it exhibits a uPA/tPA selectivity of 20 (59), the PrAg-U2 protein is expected to be a specific substrate of uPA. uPA/tPA selectivity of the sequence SGRSA can be further enhanced by placing lysine in the P1 position (59). Thus, the sequence PGSGKSA, which exhibits a uPA/tPA selectivity of 121 (59), was selected for insertion into the mutant PrAg protein PrAg-U3, which was expected to have an even higher uPA selectivity than PrAg-U2. Ke et al. (59) further showed that the P3 and P4 residues were the primary determinants of the ability of a substrate to discriminate between tPA and uPA. Thus, substitution of both the P4 glycine and the P3 serine of the most labile uPA substrate (GSGRSA) with glutamine and arginine, respectively, decreased the uPA/tPA selectivity by a factor of 1200 and yielded a tPA-selective substrate (59). Based on that result, we constructed the mutant PrAg protein PrAg-U4 containing the sequence PQRGRSA, so as to produce a tPA-specific substrate. A mutant PrAg protein PrAg-U7 was also constructed, in which ¹⁶⁴RKKR¹⁶⁷ was replaced by the sequence PGG. PrAg-U7 is not expected to be cleaved by any known protease and was used as a control protein in this study. The designations of the mutant PrAg proteins along with the expected properties based on the study of Ke et al. (59) are summarized in Table 2.

Plasmids encoding these mutant PrAg proteins were constructed by a modified overlap PCR method, cloned into the Escherichia coli-Bacillus shuttle vector pYS5, and expressed in B. anthracis UM23C₁₋₁. The proteins were secreted into the culture supernatants at 20-50 mg/liter. The mutant PrAg proteins were concentrated and purified by MonoQ chromatography to one prominent band at the expected molecular mass of 83 kDa, which co-migrated with native PrAg in SDS-PAGE. Thus, using a production protocol that is now standard in this laboratory, these mutant PrAg proteins could be expressed and purified easily in high yield and purity.

To verify that the mutant PrAg proteins had the expected susceptibility to cleavage by proteases, they were incubated separately with uPA, tPA, and a soluble form of furin. As expected, these mutant PrAg proteins were not cleaved by furin, whereas the native PrAg was cleaved by furin to produce the active PrAg63 product. The cleavage by furin after the ¹⁶⁴RKKR¹⁶⁷ sequence was confirmed by amino-terminal sequencing of the resulting PrAg63. The relative susceptibilities of the mutant PrAg proteins to cleavage by uPA and tPA agreed closely with what was predicted from the phage display data used in their design. In particular, uFA cleaved PrAg-U2 very efficiently but was less active on PrAg-U3. Moreover, PrAg-U2 was quite resistant to tPA, with just trace amounts being cleaved even with a 3-h incubation period. PrAg-U3 was even more resistant to tPA, in that no cleavage could be detected at any time point. These results showed the high uPA specificity for these two mutant PrAg proteins. In contrast, PrAg-U4 was a very weak substrate for UPA, but a good substrate for tPA. The cleavage of PrAg-U2 and PrAg-U3 at the predicted peptide bonds by uPA and that of PrAg-U4 by tPA was confirmed by amino-terminal sequencing of the resulting PrAg63s. PrAg-U7 and PrAg-U1 were both completely resistant to uPA and tPA. Native PrAg was completely resistant to tPA but was slightly cleaved by uPA at the furin recognition site. When we replaced the furin site with the sequence PGG to produce PrAg-U7, the protein was completely resistant to uPA.

PrAg-U2 and PrAg-U3 Selectively Kill uPAR-expressing Tumor Cells—To test the hypothesis that PrAg-U2 and PrAg-U3 would selectively kill uPAR-overexpressing tumor cells, cytotoxicity assays were performed with two human tumor cell lines, cervix adenocarcinoma HeLa and melanoma A2058. The non-tumor human kidney cell line 293 was used as a control. Expression of uPAR by these two tumor cell lines but not by 293 cells was reported previously (65) and was confirmed in this study by performing a pro-uPA binding and processing assay. In the presence of plasminogen, both HeLa and A2058 cells bound pro-uPA and processed it to the active, two-chain form, as identified by the uPA B-chain antibody. In contrast, the uPAR non-expressing 293 cells showed only a weak binding and failed to convert pro-uPA to two-chain uPA.

Cytotoxicity of native PrAg and the mutant PrAg proteins to these cells was measured in 96-well plates. In tumor tissues, cancer cells typically overexpress uPAR, whereas either the cancer cells or the adjacent stromal cells express pro-uPA, which is activated on the cancer cell surface after binding to uPAR. We showed that HeLa and A2058 cells did not express pro-uPA under the current culture condition. Therefore, in the cytotoxicity assay, 100 ng/ml pro-uPA was added to the tumor cells to mimic the role of pro-uPA secreted in tumor tissues in vivo. We also added 1 μg/ml Gluplasminogen, because plasminogen is present in high concentration (1.5-2.0 μM) in plasma and interstitial fluids and is required for uPAR-dependent conversion of pro-uPA to active uPA. The cells were then incubated with the native or the mutant PrAg proteins combined with FP59 for 6 h, and cell viability was measured after 48 h. The results showed that the uPAR non-expressing 293 cells were sensitive to native PrAg in a dose-dependent manner but were completely resistant to killing by all the mutant PrAg proteins. In contrast, the uPAR-expressing HeLa and A2058 cells were highly susceptible to killing by native PrAg, PrAg-U2, and PrAg-U3, were less susceptible to PrAg-U4, and were completely resistant to PrAg-U1 and PrAg-U7. The EC₅₀ values (concentrations needed to kill half of the cells) for native PrAg and the mutant PrAg proteins are summarized in Table 1. The rank order of the cytotoxicity of these PrAg proteins correlated well with the uPA cleavage profiles, strongly suggesting that the cytotoxicity observed was dependent on the uPA activity generated by the uPAR-expressing tumor cells. The selective cytotoxicity of the mutant PrAg proteins for the tumor cells was retained when the experiments were repeated in medium containing 10% fetal calf serum (data not shown). This indicates that serum proteases do not activate the PrAg proteins, nor do serum protease inhibitors block proteolytic cleavage of mutant PrAg proteins by the cell surface proteases. To simplify further analysis, all subsequent experiments were performed in serum-free medium.

Killing of uPAR-Expressing Tumor Cells by the Mutant PrAg Proteins is Dependent on the Integrity of the Cell Surface-associated Plasminogen System

To verify that the cytotoxicity of the mutant PrAg proteins was dependent on the cell surface-associated plasminogen activation system, we first tested the role of pro-uPA in the action of the mutant PrAg proteins. When the cytotoxicity experiments were repeated without addition of pro-PA, the mutant PrAg proteins PrAg-U2, PrAg-U3, and PrAg-U4 were not toxic to HeLa and A2058 cells, whereas native PrAg retained the same cytotoxicity. Furthermore, the killing of HeLa cells by PrAg-U2 was directly dependent on the concentration of pro-uPA added. No cytotoxicity was detected in the absence of pro-uPA, whereas substantial killing occurred at a pro-uPA concentration of only 12.5 ng/ml. These data prove that the toxicity of these mutant PrAg proteins to the tumor cells is dependent on the presence and activation of pro-uPA. Within tissues, the pro-uPA bound to cell surface uPAR is usually produced by neighboring cells or adsorbed from plasma. Few types of cultured cells produce both cell surface uPAR and secreted pro-uPA. One example is the Lewis lung carcinoma cell line LL3, which produces both proteins (66-69). Therefore, it was expected that the LL3 line would be susceptible to PrAg-U2 even in the absence of added pro-uPA, and this was confirmed. Killing was especially pronounced when exposure to toxin was extended to 48 h.

We next assessed the binding and proteolytic activation of pro-uPA and PrAg-U2 on uPAR-expressing and uPAR non-expressing cells. uPAR-expressing HeLa cells and non-expressing 293 cells were incubated with 1 pg/ml each of pro-uPA and PrAg-U2 in the absence or presence of plasminogen, PAI-1, and tranexamic acid for the various durations of time. Thereafter, cell lysates and conditioned media were examined by Western blotting to detect the binding and processing of pro-uPA and PrAg-U2. uPAR-expressing HeLa cells proteolytically activated pro-uPA, with active uPA accumulating both on the cell surface and in the medium. In contrast, the uPAR non-expressing 293 cells bound weakly but could not cleave pro-uPA, and only trace amounts of active uPA accumulated in the medium. The activation of pro-uPA by HeLa cells was completely blocked by PAI-1, providing further evidence that uPA is activated on the cell surface through a reciprocal activation loop involving pro-uPA and plasminogen. Activation of PrAg-U2 on the HeLa cell surface, determined by the production of the processed form PrAg 63 and the formation of SDS-stable PrAg 63 oligomer, exactly matched the activation profile of pro-uPA on the cell surface. In particular, when the activation of pro-uPA was blocked by PAI-1, PrAg-U2 activation was blocked in parallel, demonstrating that the activation of PrAg-U2 on the HeLa cell surface required the activation of pro-uPA. As expected, the uPAR non-expressing 293 cells could process neither pro-uPA nor PrAg-U2. As a control experiment, we showed that HeLa and 293 cells could process native PrAg (by furin), and this could not be inhibited by PAI-1. The effect of PAI-1 on cytotoxicity of native PrAg and PrAg-U2 was also assessed. As expected, PAI-1 conferred strong protection to HeLa cells from PrAg-U2 plus FP59, but not from native PrAg plus FP59.

Although active uPA could also be detected in the conditioned medium of HeLa cells, just a trace amount of PrAg-U2 was activated in the medium, indicating that the coincident binding of PrAg-U2 and uPA on the cell surface facilitated the activation of PrAg-U2 by uPA. To further support this, we also assessed the effects of PrAg-U7, the uncleavable PrAg variant, on the binding and processing of PrAg-U2 by HeLa cells. We showed that PrAg-U2 binding and processing on the HeLa cell surface was completely blocked by the excess amount (200-fold) of PrAg-U7, and the cytotoxicity of PrAg-U2 to HeLa cells was blocked in parallel. In agreement with this, the selective cytotoxicity of PrAg-U2 to uPAR-expressing HeLa cells was retained even in a co-culture with the uPAR non-expressing 293 cells, whereas native PrAg killed both cell types. The fact that PrAg-U2 activated on HeLa cells did not spill over and cause killing of the bystander 293 cells suggests that the specificity toward tumor cells may be retained in vivo.

The involvement of cell surface-bound plasminogen in the activation of pro-uPA and PrAg-U2 was investigated by the use of tranexamic acid, which inhibits the binding of plasminogen to the cell surface (11, 70). Pretreatment of cells with 1 mM tranexainic acid strongly inhibited the activation of pro-uPA and PrAg-U2 but not the activation of native PrAg. The involvement of cell surface-bound plasminogen in the cascade activation of pro-uPA and PrAg-U2 was further demonstrated by comparing the effects of two plasmin inhibitors, α₂-antiplasmin and aprotinin. Aprotinin, which can inhibit the activity of plasmin both on the cell sur-face and in solution (10, 11), protected HeLa cells from killing by PrAg-U2 plus FP59. In contrast, α₂-antiplasmin, which is an inefficient inhibitor of cell surface-bound plasmin (10, 11), could not protect the cells. Aprotinin and cm2-antiplas-mm had no effect on the killing of cells by native PrAg plus FP59.

We next addressed the role of uPAR in the cytotoxicity of PrAg-U2 to the uPAR-expressing HeLa cells by preincubating cells with two reagents that specifically block the binding of uPA to its receptor. ATF, the amino-terminal fragment of uPA, competes with pro-uPA for binding to uPAR. It protected the tumor cells from killing by PrAg-U2 plus FP59 in a dose-de-pendent manner but had no effect on killing by native PrAg plus FP59. Similarly, the monoclonal uPAR antibody R3 that specifically blocks the binding of pro-uPA to uPAR also protected the tumor cells from killing by the uPA-activated cytotoxin but had no effect on the killing of cells by native PrAg. These results demonstrate that the activation of PrAg-U2 and the tumor cell killing was dependent on the binding of pro-uPA to uPAR. Taken together, we conclude that the cytotoxicity of the uPA-activated PrAg proteins to uPAR-expressing tumor cells was dependent on the integrity of the cell surface-associated plasminogen activation system.

PrAg-U4 is Toxic to tPA-Expressing Cells Whereas PrA.g-U2 and PrAg-U3 are Only Weakly Toxic

Because PrAg-U4 is the mutant PrAg that is most susceptible to cleavage by tPA, we expected it to be toxic to tPA-expressing cells. To test this hypothesis, cytotoxicity assays were performed on two tPA-expressing cells, human Bowes melanoma cells and primary human umbilical vein endothelial cells (HUVEC). The expression of tPA by these cells was demonstrated by Western blotting of culture supernatants using a polyclonal antibody against human tPA. The cytotoxicity assay was done in serum-free DMEM without addition of pro-uPA and Gluplasminogen. Different concentrations of native PrAg, PrAg-U2, PrAg-U3, and PrAg-U4 combined with 50 ng/ml FP59 were incubated with cells for 12 h, and cell viability was measured at 48 h. PrAg-U4 was toxic to the two tPA-expressing cells, whereas PrAg-U2 and PrAg-U3 showed very low toxicity. The EC₅₀ values of the PrAg proteins to these tPA-expressing cells are summarized in Table 1. These and the above results clearly show that these mutant PrAg proteins, PrAg-U2, PrAg-U3, and PrAg-U4, have differential cytotoxicity to the uPA/uPAR and tPA-expressing cells.

Discussion

The results reported here clearly demonstrate that the cytotoxicity of these mutant PrAg proteins is dependent on the tumor cell surface-associated plasminogen activation system, and in particular requires the presence of pro-uPA and its receptor uPAR on the tumor cell surface. The tPA-specific mutant PrAg protein, PrAg-U4, is useful for targeting of tumors overexpressing tPA such as melanomas (78, 79).

The construction of cell-type-specific cytotoxic anthrax fusion proteins as theraputic agents have focused on modifying or replacing domain 4 with new targeting ligands (63, 81). However, the present invention provides a more effective and novel approach for improving specificity of diagnostic and therapeutic agents by combining two conceptually distinct targeting strategies in a single PrAg protein. Thus, a PrAg protein that is both retargeted to a tumor cell surface protein and dependent on the cell surface plasminogen activation system may achieve therapeutic effects that are more sensitive and highly specific while being free of the adverse effects observed with many of the existing immunotoxins and diagnostic effects that are more sensitive and highly specific.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

### REFERENCES

1. Smith, H., and J. L. Stanley. 1962. Purification of the third factor of anthrax toxin. J. Gen. Microbiol. 29:517-521.
2. Leppla, S. H. 1991. The anthrax toxin complex, p. 277-302. In J. E. Alouf and J. H. Freer (ed.), Sourcebook of bacterial protein toxins. Academic Press, London, United Kingdom.
3. Leppla, S. H. 1995. Anthrax toxins, Handb. Nat. Toxins 8:543-572.
4. Escuyer, V., and R. J. Collier. 1991. Anthrax protective antigen interacts with a specific receptor on surface of CHO-K1 cells. Infect. Immun. 59:3381-3386.
5. Klimpel, K. R., S. S. Molloy, G. Thomas, and S. H. Leppla. 1992. Anthrax toxin protective antigen is activated by a cell-surface protease with the sequence specificity and catalytic properties of furin. Proc. Natl. Acad. Sci. USA 89:10277-10281.
6. Molloy, S. S., P. A. Bresnahan, S. H. Leppla, K. R. Klimpel, and G. Thomas. 1992. Human furin is a calcium-dependent serine endoprotease that recognizes the sequence Arg-X-X-Arg and efficiently cleaves anthrax toxin protective antigen. J. B. Chem. 267:16396-16402.
7. Klimpel, K. R., Arora, N., and Leppla, S. H. 1994. Mol. Microbiol. 13:1094-1100.
8. Milne, J. C., D. Furlong, P. C. Hanna, J. S. Wall, and R. J. Collier. 1994. Anthrax protective antigen forms oligomers during intoxication of mammalian cells. J. Biol. Chem. 269: 20607-20612.
9. Benson, E. L., P. D. Huynh, A. Finkelstein, and R. J. Collier. 1998. Identification of residues lining the anthrax protective antigen channel. Biochemistry 37:3941-3948.
10. Leppla, S. H., A. M. Friedlander, and E. Cora. 1988. Proteolytic activation of anthrax toxin bound to cellular receptors, p. 111-112. In F. Fehrenbach, J. E. Alouf, P. Falmagne, W. Goebel, J. Jeljaszewicz, D. Jurgen, and R. Rappuoli (ed.), Bacterial protein toxins. Gustav Fischer, New York, N.Y.
11. Gordon, V. M., Leppla, S. H., and Hewlett, E. L. 1988. Inhibitors of receptor-mediated endocytosis block the entry of Bacillus anthracis adenylate cyclase toxin but not that of Bordetella pertussis adenylate cyclase toxin. Infect. Immun. 56: 1066-1069.
12. Friedlander, A. M. 1986. Macrophages are sensitive to anthrax lethal toxin through an acid-dependent process. J. Biol. Chem. 261:7123-7126.
13. Beal, F. A., Taylor, M. J., Thorne, C. B. 1962. Rapid lethal effect in rats of a third component found upon fractionating the toxin of Bacillus anthracis. J. Bacteriol. 83:1274-1280.
14. Ezzell, J. W., Ivins, B. E., Leppla, S. H. 1984. Immunoelectrophoretic analysis, toxicity, and kinetics of in vitro production of the protective antigen and lethal factor components of bacillus anthracis toxin. Infect. Immun. 45:761-767.
15. Hanna, P. C., Kouchi, S., Collier, R. J. 1992. Biochemical and physiological changes induced by anthrax lethal toxin in J774 macrophage-like cells. Mol. Biol. Cell. 3:1267-1277.
16. Duesbery N S, Webb C P, Leppla S H, Gorgon V M, Klimpel K R, Copeland T D, Ahn N G, Oskarsson M K, Fukasawa K, Paull K D, Vande Woude G F. 1998. Proteolytic inactivation of MAP-Kinase-Kinase by anthrax lethal factor. Science 280:734-737.
17. Vitale G, Pellizzari R, Recchi C, Napolitani G, Mock M, Montecucco C. 1998. Anthrax lethal factor cleaves the N-terminus of MAPKKs and induces tyrosine/threonine phosphorylation of MAPKs in cultured macrophages. Biochem. Biophys. Res. Commun. 248:706-711.
18. Leppla S H. 1982. Anthrax toxin edema factor: a bacterial adenylate cyclase that increases cyclic AMP concentrations of eukaryotic cells. Proc. Natl. Acad. Sci. USA. 79:3162-3166.
19. Quinn C P, Singh Y, Klimpel K R, Leppla S H. 1991. Functional mapping of anthrax toxin lethal factor by in-frame insertion mutagenesis. J. Biol. Chem. 166:20124-20130.
20. Arora N, Klimpel K R, Singh Y, Leppla S H. 1992. Fusions of anthrax toxin lethal factor to the ADP-ribosylation domain of Pseudomonas exotoxin A are potent cytotoxins which are translocated to the cytosol of mammalian cells. J. Biol. Chem. 267:15542-15548.
21. Arora N, Leppla S H. 1993. Residues 1-254 of anthrax toxin lethal factor are sufficient to cause cellula uptake of fused polypeptides. J. Biol. Chem. 268:3334-3341.
22. Petosa C, Collier R J, Klimpel K R, Leppla S H, Liddington R C. 1997. Crystal structure of the anthrax toxin protective antigen. Nature 385:833-838.
23. Birkedal-Hansen H. 1995. Proteolytic remodeling of extracellular matrix. Curr Opin Cell Biol 7:728-735.
24. Alexander C M, Hansell E J, Behrendtsen O, Flannery M L, Kishnani N S, Hawkes S P, Werb Z. 1996. Expression and function of matrix metalloproteases and their inhibitors at maternal-embryonic boundary during mouse embryo implantation. Development 122:1723-1736.
25. Schnaper H W, Grant D S, Stetler Stevensen W G, Fridman R, D'Orazi G, Murphy A N, Bird R E, Hoythya M, Fuerst T R, Frence D L, Quigley J P, Kleinman H K. 1993. Typr IV collagenases and TIMPs modulate endothelial cell morphogenesis in vitro. J Cell Physiol 156:235-246.
26. Brooks P C, Silletti S, von Schalacha T L, Friedlander M, Cheresh M. 1998. Disruption of angiogenesis by PEX, a noncatalytic metalloprotease fragment with integrin binding activity. Cell 92:391-400.
27. Crawford H C, and Matrisian L M. 1995. Tumor and stromal expression of matrix metalloproteases and their role in tumor progression. Invasion Metastasis. 14:234-245.
28. Garbisa S, Pozzati R, Mushel R, Saffiotti U, Ballin M, Goldfarb R, Khoury G, Liotta L A. 1987. Secretion of type IV collagenolytic protease and metastatic phenotype: induction by transfection with c-Ha-ras but not c-Ha-ras+Ad2-E1a. Cancer Res., 47:1523-1528.
29. Himelstein B P, Canete-Soler R, Bernhard E J, Dilks D W and Muschel R L. 1995. Metalloproteases in tumor progression: the contribution of MMP-9. Invest. Methods. 14:246-258.
30. Juarez J, Clayman G, Nakajima M, Tanabe K K, Saya H, Nicolson G L, Boyd D. 1993. Role and regulation expression of 92-kD type IV collagenase (MMP-9) in 2 invasive squamous-cell-carcinoma cell lines of the oral cavity. Int. J. Cancer. 55:10-18.
31. Kohn E C, and Liotta L A. 1995. Molecular insights into cancer invasion: strategies for prevention and intervention. Cancer Res. 55:1856-1862
32. Levy A T, Cioce V, Sobel M E, Garbisa S, Grigioni W, Liotta L A and Stetler-Stevensen W G. 1991. Increased expression of 72 kDa type IV collagenase in human colonic adenocarcinoma. Cancer Res. 51:439-444.
33. Mignatti P, and Rifkin D B. 1993. Biology and biochemistry of proteases in tumor invasion. Physiol. Rev. 73-161-195.
34. Montgomery A M, De Clerck Y A, Langley K E, Reisfeld B M and Mueller B M. 1993. Melanoma-mediated dissolution of extracellular matrix: contribution of urokinase-dependent and matrix metalloprotease-dependent proteolytic pathways. Cancer Res. 53:693-700.
35. Stetler-Stevenson W G, Aznavoorian S, Liotta L A. 1993. Annu Rev Cell Biol 9:541-573.
36. Stetler-Stevenson W G. 1995. Progelatinase A activation during tumor cell invasion. Invest. Methods. 14:4664-4671.
37. Davidson B, Goldberg I, Kopolovic J, Lerner-Geva L, Gotlieb W H, Ben-Baruch G, Reich R. 1999. MMP-2 and TIMP-2 expression correlates with poor prognosis in cervical carcinoma—a clinicopathologic study using immunohistochemistry and mRNA in situ hybridization. Gynecol. Oncol. 73:372-382.
38. Webber M M, Trakul N, Thraves P S, Bello-DeOcampo D, Chu W W, Storto P D, Huard T K, Rhim J S, Williams D E. 1999. A human prostatic stromal myofibroblast cell line WPMY-1: a model for stromal-epithelial interactions in prostatic neoplasia. Carcinogenesis 20:1185-1192.
39. Johansson N, Vaalamo M, Grenman S, Hietanen S, Klemi P, Saarialho-Kere U, Kahari V M. 1999. Collagenase-3 (MMP-13) is expressed by tumor cells in invasive vulvar squamous cell carcinomas. Am J Pathol 154:469-480.
40. Ries C, Loher F, Zang C, Lsmair M G, Petrides P E. 1999. Matrix metalloprotease production by bone mononuclear cells from normal individuals and patients with acute and chronic myeloid leukemia or myelodysplastic syndromes. Clin Cancer Res. 5:1115-1124.
41. Zeng Z S, Gohen A M, Guillem J G. 1999. Loss of basement membrane type IV collagen is associated with increased expression of metalloproteases 2 and 9 (MMP-2 and MMP-9) during human colorectal tumorigenesis. Carcinogenesis 20:749-755.
42. Gokaslan Z L, Chintala S K, York J E, Boyapati V, Jasti S, Sawaya R, Fuller G, Wildrick D M, Nicolson G L, Rao J S. 1998. Expression and role of matrix metalloproteases MMP-2 and MMP-9 in human spinal column tumors. Clin Exp Metastasis 16:721-728.
43. Forsyth P A, Wong H, Laing T D, Rewcastle N B, Morris D G, Muzik H, Leco K J, Johnston R N, Brasher P M, Sutherland G, Edwards D R. 1999 Gelatinase-A (MMP-2), gelatinase-B (MMP-9) and membrane type matrix metalloprotease-1 (MT1-MMP) are involved in different aspects of the pathophysiology of malignant gliomas. Br J Cancer 79:1828-1835.
44. Ozdemir E, Kakehi Y, Okuno H, Yoshida O. 1999. Role of matrix metalloprotease-9 in the basement membrane destruction of superficial urothelial carcinomas. J Urol 161:1359-1363.
45. Nomura H, Sato H, Seiki M, Mai M and Okada Y. 1995. Expression of membrane-type matrix metalloprotease in human gastric carcinomas. Cancer. Res. 55:3263-3266.
46. Okada Y, Bellocq J P, Rouyer N, Chenard M P, Rio M C, Chambon P and Basset P. 1995. Membrane-type matrix metalloprotease (MT-MMP) gene is expressed in stromal cells of human colon, breast, head and neck carcinomas. Proc. Natl. Acad. Sci. USA. 92:2730-2734.
47. Sato H, Takino T, Okada Y, Cao J, Shinagawa A, Yamamoto E and Seiki M. 1994. A matrix metalloprotease expressed on the surface of invasive tumor cells. Nature. 370:61-65.
48. Chen W T, Wang J Y. 1999. Specialized surface protrusions of invasive cells, invadopodia and lamellipodia, have differential MT1-MMP, MMP-2 and TIMP-2 localization. Ann N Y Acad Sci 878:361-371.
49. Sato T, Lwai M, Sakai T, Sato H, Seiki M Mori Y Ito A. 1999. Enhancement of membrane-type 1-matrix metalloprotease (MT 1-MMP) production and sequential activation of progelatinase A on human squamous carcinoma cells co-cultured with human dermal fibroblasts. Br J Cancer 80:1137-43.
50. Polette M, Birembaut P. 1998. Membrane-type metalloproteases in tumor invasion. Int J Biochem cell Biol. 30:1195-1202.
51. Kitagawa Y, Kunimi K, Ito H, Sato H, Uchibayashi T, Okada Y, Seiki M, Namiki M. Expression and tissue localization of membrane-types 1, 2, and 3 matrix metalloproteases in human urothelial carcinomas. J Urol. 160: 1540-1545.
52. Nakada M, Nakamura H, Ikeda E, Fujimoto N, Yamashita J, Sato H, Seiki M, Okada Y. 1999. Expression and tissue localization of membrane-types 1, 2, and 3 matrix metalloproteases in human Astrocytic tumors. 1999. Am J Pathol. 154:417-428.
53. Sato H, Okada Y, Seiki M. 1997. Membrane-type matrix metalloproteases (MT-MMPs) in cell invasion. Thromb Haemost 78:497-500.
54. Chambers A F and Matrisian L M. 1997. Changing views of the role of matrix metalloproteases in metastasis. J Natl Cancer Inst 89:1260-1270.
55. Arribas J, Goodly L, Vollmer P, Kishimoto T K, Rose-John S, Massague J. 1996. Diverse cell surface protein ectodomains are shed by a system sensitive to metalloprotease inhibitors. J Biol Chem 271:11376-11382.
56. Suzuki M, Raab G, Moses M A, Fernandez Calif., Klagsbrun M. 1997. Matrix metalloprotease-3 releases active heparin-binding EGF-like growth factor by cleavage at a specific juxtamembrane site. J Biol Chem 272:31730-31737.
57. Springman E B, Angleton E L, Birkedal-Hansen H, Van Wart H E. 1990. Proc Natl Acad Sci USA 87:364-368.
58. Murphy G, Stanton H, Cowell S, Butler G, Knauper V, Atkinson S, Gavrilovic J. 1999. Mechanisms for pro matrix metalloprotease activation. APMIS 107:38-44.
59. Birkedal-Hansen H, Moose W G, Bodden M K, Windsor L J, Birkedal-Hansen B, DeCarlo A, Englar J A. 1993. Crit Rev Oral Biol Med 4:197-250.
60. Brown P D. 1995. Matrix metalloprotease inhibitors: a novel class of anticancer agents. Adv Enzyme Regul 35:293-301.
61. Wojtowicz-Praga S, Torri J, Johnson M, Steen V, Marshall J, Ness E, Dickson R, Sale M, Rasmussen H S, Chiodo T A, Hawkins M J. 1998. Phase I trial of Marimastat, a novel matrix metalloprotease inhibitor, administered orally to patients with advanced lung cancer. J Clin Oncol 16:2150-2156.
62. Drummond A H, Beckett P, Brown P D, Bone E A, Davidson A H, Galloway W A, Gearing A J, Huxley P, Laber D, McCourt M, Whittaker M, Wood L M, Wright A. 1999. Preclinical and clinical studies of MMP inhibitors in cancer. Ann N Y Acad Sci 30:228-235.
63. Preparation of soluble form of furin
64. Grobelny D, Poncz L, Galardy R E. 1992. Inhibition of human skin fibroblast collagenase, thermolysin, and Pseudomonas Aeruginosa Elastase by peptide hydroxamic acids. Biochemistry 31:7152-7154.
65. Singh Y, Chaudhary V K, Leppla S H. 1989. A deleted variant of Bacillus anthracis protective antigen is non-toxic and blocks anthrax toxin action in vivo. J Biol Chem 264:19103-19107.
66. Varughese M, Teixeira A V, Liu S, Leppla S H. 1999. Identification of a receptor-binding region within domain 4 of the protective antigen component of anthrax toxin. Infect Immun 67:1860-1865.
67. Netzel-Arnett S, Fields G B, Birkedal-Hansen H, Van Wart H E. 1991. J Biol Chem 266: 6747-6755.
68. Netzel-Arnett S, Sang Q, Moore W G I, Navre M, Birkedal-Hansen H, Van Ward H E. 1993. Comparative sequence specificities of human 72- and 92-kDa gelatinases (type IV collagenases) and PUMP (matrilysin). Biochemistry 32:6427-6432.
69. Will H, Atkinson S J, Butler G S, Smith B, Murphy G. 1996. The soluble catalytic domain of membrane type 1 matrix metalloprotease cleaves the propeptide of progelatinase A and initiates autoproteolytic activation. J Biol Chem 271:17119-17123.
70. Mazzieri R, Masiero L, Zanetta L, Monea S, Onisto M, Garbisa S, Mignatti P. 1997. Control of type IV collagenase activity by components of the urokinase-plasmin system: a regulatory mechanism with cell-bound reactants. EMBO J. 16:2319-2332.
71. Brooks P C, Stromblad S, Sanders L C, von Schalscha T L, Aimes R T, Stetler-Stevensen W G, Quigley J P, Cheresh D A. 1996. Localization of matrix metalloprotease mmp-2 to the surface of invasive cells by interaction with integrin αvβ3. Cell 85: 683-693.
72. Tu Q, Stamenkovic I. 1999. Localization of matrix metalloprotease 9 to the cell surface provides a mechanism for CD44-mediated tumor invasion. Gene Development 13:35-48.
73. Thrush G R, Lark L R, Clinchy B C, Vitetta E S. 1996. Immunotoxins: an update. Annu Rev Immunol 14:49-71.

1. A method for imaging the activity of a specific extracellular protease expressed by a cell, the method comprising the steps of:
(a) contacting a cell with a mutant anthrax protective antigen (μPrAg), under conditions where the μPrAg binds to a cell surface receptor expressed by the cell and is cleaved by a specific extracellular protease expressed by the cell,
wherein the μPrAg comprises a domain for binding the cell surface receptor, and comprises a protease cleavage site that is cleaved by the specific extracellular protease and is in place of the furin cleavage site of the native anthrax protective antigen (PrAg);
(b) contacting the cell with a ligand linked to a detectable moiety, under conditions where the ligand specifically binds to the cleaved μPrAg of step (a), thereby, forming a ligand-μPrAg complex; and
(c) imaging the detectable moiety linked to the ligand bound in the ligand-μPrAg complex of step (b) and, thereby, generating an image of the detectable moiety,
wherein the image of the detectable moiety is indicative of the activity of the specific extracellular protease.

2. The method of claim 1, wherein steps (a), (b), and (c) are performed in vivo.

3. The method of claim 1, wherein steps (a), (b), and (c) are performed ex vivo.

4. The method of claim 1, wherein steps (a), (b), and (c) are performed in vitro.

5. The method of claim 1, wherein the specific extracellular protease is a matrix metalloprotease (MMP) or plasminogen activator (PA).

6. The method of claim 5, wherein the matrix metalloprotease (M) is MMP-2 (gelatinase A), MMP-9 (gelatinase B), or membrane-type 1 MMP (MT 1-MMP).

7. The method of claim 5, wherein the plasminogen activator is tissue plasminogen activator (tPA) or urokinase plasminogen activator (uPA.).

8. The method of claim 1, wherein the protease cleavage site of the μPrAg is encoded by an amino acid sequence selected from a group consisting of GPLGMLSQ, GPLGLWAQ, PCPGRVVGG, PGSGRSA, PGSGKSA, PQRGRSA, PCPGRVVGG, PGSGRSA, PGSGKSA, PQRGRSA, GPLGMLSQ, and GPLGLWAQ.

9. The method of claim 1, wherein the ligand is a protein that specifically binds to the cleaved μPrAg of step (a).

10. The method of claim 1, wherein the ligand is a noncytotoxic mutant lethal factor or noncytotoxic mutant endema factor that specifically binds to the cleaved μPrAg of step (a).

11. The method of claim 1, wherein the ligand is a noncytotoxic derivative of lethal factor or noncytotoxic derivative of endema factor that specifically binds to the cleaved μPrAg of step (a).

12. The method of claim 1, wherein the ligand is FP59.

13. The method of claim 1, wherein the ligand is LFN.

14. The method of claim 9, wherein the protein is a μPrAg antibody.

15. The method of claim 1, wherein the ligand-μPrAg complex of step (b) is translocated into the cell.

16. The method of claim 1, wherein the ligand-μPrAg complex of step (b) remains on the cell surface.

17. The method of claim 1, wherein the imaging is by magnetic resonance, radioscintigraphy, positron emission tomography, computed tomography, near-infrared fluorescence, X-ray, ultra sound, ultraviolet light, or visible light.

18. The method of claim 1, wherein the detectable moiety is a radionuclide, biotin moiety, enzyme, metal ion, chromophore, or fluorophore.

19. The method of claim 1, wherein the cell is a cancer cell, a lymphocyte, a neuron, a cardiovascular cell, or an inflammatory cell.

20. The method of claim 1, wherein the cell is a human cell.

21. The method of claim 2, wherein the cell is in a mammal.

22. The method of claim 21, wherein the mammal is a rodent or human.

23. The method of claim 22, wherein prior to step (a), a composition comprising the μPrAg and ligand are administered to the rodent or human.

24. The method of claim 22, wherein the activity of the protease is a diagnostic indicator of a disease or undesirable physiological condition correlated with the activity.

25. The method of claim 24, wherein the disease is cancer, chronic inflammation, acute inflammation, autoimmune disease, cardiovascular disease, infection, or a neurological disorder.

26. The method of claim 24, wherein the condition is inflammation.

27. The method of claim 24, wherein the condition is cancer.

28. A method for assaying for an inhibitor of an extracellular protease expressed by a cell, the method comprising the steps of:
(a) contacting a cell with a potential inhibitory compound and a mutant anthrax protective antigen (μPrAg), under conditions where the μPrAg binds to a cell surface receptor expressed by the cell and is cleaved by a specific extracellular protease expressed by the cell,
wherein the μPrAg comprises a domain for binding the cell surface receptor, and comprises a protease cleavage site that is cleaved by the specific extracellular protease and is in place of the furin cleavage site of the native anthrax protective antigen (PrAg);
(b) contacting the cell with a ligand linked to a detectable moiety, under conditions where the ligand specifically binds to the cleaved μPrAg of step (a), thereby, forming a ligand-μPrAg complex; and
(c) imaging the detectable moiety linked to the ligand bound in the ligand-μPrAg complex of step (b) and, thereby, generating an image of the detectable moiety,
wherein the image of the detectable moiety is indicative of the activity of the specific extracellular protease, thereby identifying inhibitors of the extracellular protease.


SEQUENCE LISTING (24 sequences)

SEQ ID NO: 1 - protein, 4 aa, Artificial Sequence
  Description of Artificial Sequencefurin cleavage site
RKKR

SEQ ID NO: 2 - protein, 8 aa, Artificial Sequence
  Description of Artificial Sequencematrix metaloprotease (MMP) substrate octapeptide in PA-L1, MMP-2 (gelatinase A) favorite sequence
GPLGMLSQ

SEQ ID NO: 3 - protein, 8 aa, Artificial Sequence
  Description of Artificial Sequencematrix metaloprotease (MMP) substrate octapeptide in PA-L2, MMP-9 (gelatinase B) favorite sequence
GPLGLWAQ

SEQ ID NO: 4 - protein, 9 aa, Artificial Sequence
  Description of Artificial Sequenceplasminogen-derived sequence in clone PrAg-U1, plasminogen activator cleavage site
PCPGRVVGG

SEQ ID NO: 5 - protein, 7 aa, Artificial Sequence
  Description of Artificial Sequencepreferred urokinase plasminogen activator (uPA) substrate sequence in clone PrAg-U2, plasminogen activator cleavage site
PGSGRSA

SEQ ID NO: 6 - protein, 7 aa, Artificial Sequence
  Description of Artificial Sequencepreferred urokinase plasminogen activator (uPA) substrate sequence in clone PrAg-U3, plasminogen activator cleavage site
PGSGKSA

SEQ ID NO: 7 - protein, 7 aa, Artificial Sequence
  Description of Artificial Sequencepreferred tissue plasminogen activator (tPA) substrate sequence in clone PrAg-U4, plasminogen activator cleavage site
PQRGRSA

SEQ ID NO: 8 - protein, 201 aa, Artificial Sequence
  Description of Artificial Sequenceflexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGPGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 9 - protein, 8 aa, Artificial Sequence
  Description of Artificial SequenceFLAG epitope tag
DYKDDDDK

SEQ ID NO: 10 - DNA, 18 bp, Artificial Sequence
  Description of Artificial Sequenceoverlap PCR 5′ primer F
aaaggagaacgtatatga

SEQ ID NO: 11 - DNA, 30 bp, Artificial Sequence
  Description of Artificial Sequenceoverlap PCR phosphorylated primer R1
ngagttcgaagatttttgttttaattctgg

SEQ ID NO: 12 - DNA, 52 bp, Artificial Sequence
  Description of Artificial Sequenceoverlap PCR mutagenic phosphorylated primer H1
ngaccattaggaatgtggagtcaaagtacaagtgctggacctacggttccag

SEQ ID NO: 13 - DNA, 21 bp, Artificial Sequence
  Description of Artificial Sequenceoverlap PCR reverse primer R2
acgtttatctcttattaaaat

SEQ ID NO: 14 - DNA, 52 bp, Artificial Sequence
  Description of Artificial Sequenceoverlap PCR mutagenic phosphorylated primer H2
ngaccattaggattatgggcacaaagtacaagtgctggacctacggttccag

SEQ ID NO: 15 - DNA, 33 bp, Artificial Sequence
  Description of Artificial Sequencemodified overlap PCR phosphorylated reverse primer R1
nggtgagttcgaagatttttgttttaattctgg

SEQ ID NO: 16 - DNA, 52 bp, Artificial Sequence
  Description of Artificial Sequencemodified overlap PCR mutagenic phosphorylated primer Hi
ngtccaggaagagtagttggaggaagtacaagtgctggacctacggttccag

SEQ ID NO: 17 - protein, 8 aa, Artificial Sequence
  Description of Artificial Sequencepeptide encoded by modified overlap PCR mutagenic phosphorylated primer Hi
CPGRVVGG

SEQ ID NO: 18 - DNA, 47 bp, Artificial Sequence
  Description of Artificial Sequencemodified overlap PCR mutagenic phosphorylated primer H2
ngaagtggaagatcagcaagtacaagtgctggacctacggcttccag

SEQ ID NO: 19 - protein, 6 aa, Artificial Sequence
  Description of Artificial Sequencepeptide encoded by modified overlap PCR mutagenic phosphorylated primer H2
GSGRSA

SEQ ID NO: 20 - DNA, 46 bp, Artificial Sequence
  Description of Artificial Sequencemodified overlap PCR mutagenic phosphorylated primer H3
ngaagtggaaaatcagcaagtacaagtgctggacctacggttccag

SEQ ID NO: 21 - protein, 6 aa, Artificial Sequence
  Description of Artificial Sequencepeptide encoded by modified overlap PCR mutagenic phosphorylated primer H3
GSGKSA

SEQ ID NO: 22 - DNA, 46 bp, Artificial Sequence
  Description of Artificial Sequencemodified overlap PCR mutagenic phosphorylated primer H4
nagagaggaagatcagcaagtacaagtgctggacctacggttccag

SEQ ID NO: 23 - protein, 6 aa, Artificial Sequence
  Description of Artificial Sequencepeptide encoded by modified overlap PCR mutagenic phosphorylated primer H4
QRGRSA

SEQ ID NO: 24 - protein, 5 aa, Artificial Sequence
  Description of Artificial Sequenceminimized optimum substrate for urokinase plasminogen activator (uPA) consensus sequence
SGRSA